(19) United States
(12) Patent Application Publication
(10) Pub. No.: US 2020/0270362 A1
(43) Pub. Date: Aug. 27, 2020

(54) MSLN TARGETING TRISPECIFIC PROTEINS AND METHODS OF USE

(71) Applicant: Harpoon Therapeutics, Inc., South San Francisco, CA (US)

(72) Inventors: Holger WESCHE, San Francisco, CA (US); Bryan D. LEMON, Mountain View, CA (US); Richard J. AUSTIN, San Francisco, CA (US); Robert B. DUBRIDGE, Belmont, CA (US)

(21) Appl. No.: 16/802,007

(22) Filed: Feb. 26, 2020

Related U.S. Application Data
(63) Continuation-in-part of application No. 15/977,988, filed on May 11, 2018.
(60) Provisional application No. 62/657,434, filed on Apr. 13, 2018.
(60) Provisional application No. 62/505,747, filed on May 12, 2017.

(51) Int. Cl. C07K 16/30; A61K 39/395; A61K 45/06; A61P 35/00

(52) U.S. Cl. CPC C07K 16/30; A61K 39/39558; A61K 2039/545; A61P 35/00; A61K 45/06

(57) ABSTRACT

Provided herein are mesothelin (MSLN) targeting trispecific proteins comprising a domain binding to CD3, a half-life extension domain, and a domain binding to MSLN. Also provided are pharmaceutical compositions thereof, as well as nucleic acids, recombinant expression vectors and host cells for making such MSLN targeting trispecific proteins. Also disclosed are methods of using the disclosed MSLN targeting trispecific proteins in the prevention, and/or treatment of diseases, conditions and disorders.

### CROSS-REFERENCE

This application is a continuation application of U.S. patent application Ser. No. 15/977,988, filed May 11, 2018, and claims the benefit of U.S. Provisional Application Nos. 62/505,747, filed on May 12, 2017, and 62/657,434, filed Apr. 13, 2018, all of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on May 11, 2018, is named 47517-720 201 SL.txt and is 293,251 bytes in size.

### BACKGROUND OF THE INVENTION

The selective destruction of an individual cell or a specific cell type is often desirable in a variety of clinical settings. For example, it is a primary goal of cancer therapy to specifically destroy tumor cells, while leaving healthy cells and tissues intact and undamaged. One such method is by inducing an immune response against the tumor, to make immune effector cells such as natural killer (NK) cells or cytotoxic T lymphocytes (CTLs) attack and destroy tumor cells.

Mesothelin (MSLN) is a GPI-linked membrane bound tumor antigen MSLN is overexpressed ovarian, pancreatic, lung and triple-negative breast cancers and mesothelioma. Normal tissue expression of MSLN is restricted to single-cell, mesothelial layers lining the pleural, pericardial, and peritoneal cavities. Overexpression of MSLN is associated with poor prognosis in lung adenocarcinoma and triple-negative breast cancer. MSLN has been used as cancer antigen for numerous modalities, including immunotoxins, vaccines, antibody drug conjugates and CAR-T cells. Early signs of clinical efficacy have validated MSLN as a target, but therapies with improved efficacy are needed to treat MSLN-expressing cancers.

### SUMMARY OF THE INVENTION

One embodiment provides a mesothelin binding trispecific protein, wherein said protein comprises
(a) a first domain (A) which specifically binds to human CD3;
(b) a second domain (B) which is a half-life extension domain; and
(c) a third domain (C) which specifically binds to MSLN,
wherein the domains are linked in the order H₂N-(A)-(C)—(B)—COOH, H₂N—(B)-(A)-(C)—COOH, H₂N—(C)—(B)-(A)-COOH, or by linkers L1 and L2. In some embodiments, the first domain comprises a variable light domain and a variable heavy domain each of which is capable of specifically binding to human CD3. In some embodiments, the first domain is humanized or human. In some embodiments, the second domain binds albumin. In some embodiments, the second domain comprises a scFv, a variable heavy domain (VH), a variable light domain (VL), a peptide, a ligand, or a small molecule. In some embodiments, the third domain comprises a VHH domain, a scFv, a VH domain, a VL domain, a non-Ig domain, a ligand, a knottin, or a small molecule entity that specifically binds to MSLN. In some embodiments, the third domain comprises a VHH domain. In some embodiments, said VHH domain comprises one or more conserved regions comprising a sequence identical to or comprising one or more amino acid residue substitutions relative to SEQ ID NO: 41, 42, 43, or 44. In some embodiments, said VHH domain comprises a conserved region comprising a sequence identical to or comprising one or more amino acid residue substitutions relative to SEQ ID NO: 41. In some embodiments, said VHH domain comprises a conserved region comprising a sequence identical to or comprising one or more amino acid residue substitutions relative to SEQ ID NO: 42. In some embodiments, said VHH domain comprises a conserved domain comprising a sequence identical to or comprising one or more amino acid residue substitutions relative to SEQ ID NO: 43. In some embodiments, said VHH domain comprises a conserved domain comprising a sequence identical to or comprising one or more amino acid residue substitutions relative to SEQ ID NO: 44. In some embodiments, said VHH domain comprises (i) a stretch of amino acids corresponding to SEQ ID NO: 41; (ii) a stretch of amino acids corresponding to SEQ ID NO: 42; (iii) a stretch of amino acids corresponding to SEQ ID NO: 43, and (iv) a stretch of amino acids corresponding to SEQ ID NO: 44. In some embodiments, said humanized VHH domain comprises the following formula: f1-r1-f2-r2-f3-r3-f4 wherein, r1 is identical to or comprises one or more amino acid residue substitutions relative to any one of SEQ ID Nos.: 134-44; r2 is identical to or comprises one or more amino acid residue substitutions relative to any one of SEQ ID Nos.: 173-183; and r3 is identical to or comprises one or more amino acid residue substitutions relative to SEQ ID Nos.: 212-222; and wherein f1, f2, f3 and f4 are framework residues.

In some embodiments, said VHH domain comprises the following formula:

f1-r1-f2-r2-f3-r3-f4

wherein, r1 is identical to or comprises one or more amino acid residue substitutions relative to SEQ ID NO: 51; r2 is identical to or comprises one or more amino acid residue substitutions relative to SEQ ID NO: 52; and r3 is identical to or comprises one or more amino acid residue substitutions relative to SEQ ID NO: 53; and wherein f1, f2, f3 and f4 are framework residues. In some embodiments, said VHH domain comprises a sequence that is at least 80% identical to a sequence selected from the group consisting of SEQ ID NOs: 1-29. In some embodiments, the third domain comprises selected sequence from the group consisting of SEQ ID NOs: 1-29. In some embodiments, the third domain is a humanized VHH domain. In some embodiments, said humanized VHH domain comprises one or more conserved regions comprising a sequence identical to or comprising one or more amino acid residue substitutions relative to SEQ ID NO: 45, 46, 47, 48, 49, or 50. In some embodiments, said VHH domain comprises a conserved region comprising a sequence identical to or comprising one or more amino acid residue substitutions relative to SEQ ID NO: 45. In some embodiments, said VHH domain comprises a conserved region comprising a sequence identical to or comprising one or more amino acid residue substitutions relative to SEQ ID NO: 46. In some embodiments, said VHH domain comprises a conserved domain comprising a sequence identical to or comprising one or more amino acid residue substitutions relative to SEQ ID NO: 47. In some embodiments, said VHH domain comprises a conserved domain comprising a sequence identical to or comprising one or more amino acid residue substitutions relative to SEQ ID NO: 48. In some embodiments, said VHH domain comprises a conserved domain comprising a sequence identical to or comprising one or more amino acid residue substitutions relative to SEQ ID NO: 49. In some embodiments, said VHH domain comprises a conserved domain comprising a sequence identical to or comprising one or more amino acid residue substitutions relative to SEQ ID NO: 50. In some embodiments, said VHH domain comprises (i) a stretch of amino acids corresponding to SEQ ID NO: 45, (ii) a stretch of amino acids corresponding to SEQ ID NO: 46, (iii) a stretch of amino acids corresponding to SEQ ID NO: 47, (iv) a stretch of amino acids corresponding to SEQ ID NO: 48, (v) a stretch of amino acids corresponding to SEQ ID NO: 49, and (vi) a stretch of amino acids corresponding to SEQ ID NO: 50. In some embodiments, said humanized VHH domain comprises the following formula:

f1-r1-f2-r2-f3-r3-f4

wherein, r1 is identical to or comprises one or more amino acid residue substitutions relative to SEQ ID NO: 54; r2 is identical to or comprises one or more amino acid residue substitutions relative to SEQ ID NO: 55; and r3 is identical to or comprises one or more amino acid residue substitutions relative to SEQ ID NO: 56; and wherein f1, f2, f3 and f4 are framework residues. In some embodiments, said third domain comprises a CDR1 sequence comprising a sequence as set forth in any one of SEQ ID Nos.: 51, 54, and 106-144. In some embodiments, said third domain comprises a CDR2 sequence comprising a sequence as set forth in any one of SEQ ID Nos.: 52, 55, and 145-183. In some embodiments, said third domain comprises a CDR2 sequence comprising a sequence as set forth in any one of SEQ ID Nos.: 53, 56, and 184-222. One embodiment provides, a mesothelin (MSLN) binding trispecific protein, comprising the sequence as set forth in SEQ ID NO: 98.

In some embodiments, the third domain comprises a sequence selected from the group consisting of SEQ ID NOs: 30-40, and 102-105. In some embodiments, the third domain binds to a human mesothelin protein comprising the sequence set forth as SEQ ID NO: 57. In some embodiments, the third domain binds to an epitope of mesothelin, wherein said epitope is located in region I, comprising amino acid residues 296-390 of SEQ ID NO: 57, region II comprising amino acid residue 391-486 of SEQ ID NO: 57, or region III comprising amino acid residues 487-598 of SEQ ID NO: 57. In some embodiments, linkers L1 and L2 are each independently selected from (GS). (SEQ ID NO: 87), (GGS)ₙ (SEQ ID NO: 88), (GGGS)ₙ (SEQ ID NO: 89), (GGSG)ₙ (SEQ ID NO: 90), (GGSGG)ₙ (SEQ ID NO: 91), or (GGGGS)ₙ (SEQ ID NO: 92), wherein n is 1, 2, 3, 4, 5, 6, 7, 8, 9, or 10. In some embodiments, linkers L1 and L2 are each independently (GGGGS)₄ (SEQ ID NO: 95) or (GGGGS)₃ (SEQ ID NO: 96). In some embodiments, the domains are linked in the order H₂N—(C)—(B)-(A)-COOH. In some embodiments, the protein is less than about 80 kDa. In some embodiments, the protein is about 50 to about 75 kDa. In some embodiments, the protein is less than about 60 kDa. In some embodiments, the protein has an elimination half-time of at least about 50 hours. In some embodiments, the protein has an elimination half-time of at least about 100 hours. In some embodiments, the protein has increased tissue penetration as compared to an IgG to the same MSLN. In some embodiments, the protein comprises a sequence selected from the group consisting of SEQ ID NO: 58-86, 98, 100, and 101.One embodiment provides a mesothelin binding trispecific protein, comprising the sequence as set forth in SEQ ID NO: 98. One embodiment provides a mesothelin binding trispecific protein, wherein said protein comprises: (a) a first domain (A) which specifically binds to human CD3; (b) a second domain (B) which is a half-life extension domain; and (c) a third domain (C) which specifically binds to MSLN, wherein the domains are linked in the order H₂N-(A)-(C)—(B)—COOH, H₂N—(B)-(A)-(C)—COOH, H₂N—(C)—(B)-(A)-COOH, or by linkers L1 and L2, wherein said third domain comprises one or more CDRs selected from the group consisting of SEQ ID Nos.: 51-56 and 106-222. In some embodiments, said third domain comprises a CDR1 comprising a sequence as set forth in any one of SEQ ID Nos.: 51, 54, and 106-144. In some embodiments, said third domain comprises a CDR2 comprising a sequence as set forth in any one of SEQ ID Nos.: 52, 55, and 145-183. In some embodiments, said third domain comprises a CDR2 comprising a sequence as set forth in any one of SEQ ID Nos.: 53, 56, and 184-222. In some embodiments, said third domain comprises a framework region 1 (f1) comprising a sequence as set forth in any one of SEQ ID Nos.: 262-300. In some embodiments, said third domain comprises a framework region (f2) sequence as set forth in any one of SEQ ID Nos.: 301-339. In some embodiments, said third domain comprises a framework region (f3) a sequence as set forth in any one of SEQ ID Nos.: 340-378. In some embodiments, the protein comprises a sequence selected from the group consisting of SEQ ID NOs: 58-86, 98, 100, and 101. In some embodiments, the protein comprises a sequence as set forth in SEQ ID NO: 98.

One embodiment provides a pharmaceutical composition comprising (i) the MSLN binding trispecific protein according to any one of the above embodiments and (ii) a pharmaceutically acceptable carrier.

One embodiment provides a process for the production of a mesothelin binding trispecific protein according to any one of the above embodiments, said process comprising culturing a host transformed or transfected with a vector comprising a nucleic acid sequence encoding a mesothelin binding trispecific protein according to any one of the above embodiments under conditions allowing the expression of the mesothelin binding trispecific protein and recovering and purifying the produced protein from the culture.

One embodiment provides a method for the treatment or amelioration of a proliferative disease, or a tumorous disease, comprising the administration of the mesothelin binding trispecific protein according to any one of the above embodiments, to a subject in need thereof. One embodiment provides a method for the treatment or amelioration of a proliferative disease, or a tumorous disease, comprising administration of a mesothelin binding trispecific protein comprising a sequence as set forth in any one of SEQ ID Nos.: 58-86, 98, 100, and 101.

In some embodiments, the subject is human. In some embodiments, the method further comprises administration of an agent in combination with the single domain mesothelin binding protein according to any one of the above embodiments. In some embodiments, the mesothelin binding trispecific protein selectively binds to tumor cells expressing mesothelin. In some embodiments, the mesothelin binding trispecific protein mediates T cell killing of tumor cells expressing mesothelin. In some embodiments, the tumorous disease comprises a solid tumor disease. In some embodiments, the solid tumor disease comprises mesothelioma, lung cancer, gastric cancer, ovarian cancer, or triple negative breast cancer. In some embodiments, the solid tumor disease is metastatic.

One embodiment provides a method for the treatment or amelioration of a proliferative disease, or a tumorous disease, comprising administration of a mesothelin binding trispecific protein comprising a sequence selected from the group consisting of SEQ ID NOs: 58-86, 98, 100, and 101. In some embodiments, the mesothelin binding trispecific protein selectively binds to tumor cells expressing mesothelin. In some embodiments, the mesothelin binding trispecific protein directs T cell killing of tumor cells expressing mesothelin. In some embodiments, the tumorous disease comprises a solid tumor disease. In some embodiments, the solid tumor disease comprises mesothelioma, lung cancer, gastric cancer, ovarian cancer, or triple negative breast cancer. In some embodiments, the solid tumor disease is metastatic.

One embodiment provides a method for the treatment or amelioration of a proliferative disease, or a tumorous disease, comprising administration of a mesothelin binding trispecific protein comprising a sequence as set forth in SEQ ID NO: 98. In some embodiments, the method comprises administering the mesothelin binding trispecific protein at a dose of up to 10 mg/kg. In some embodiments, the protein is administered once a week. In some embodiments, the protein is administered twice per week. In some embodiments, the protein is administered every other week. In some embodiments, the protein is administered every three weeks.

One embodiment provides a method for the treatment or amelioration of a proliferative disease, or a tumorous disease, comprising administration of a mesothelin binding trispecific protein comprising a sequence as set forth in any one of SEQ ID NOs: 58-86, 98, 100, and 101. In some embodiments, the method comprises administering the mesothelin binding trispecific protein at a dose of up to 10 mg/kg. In some embodiments, the protein is administered once a week. In some embodiments, the protein is administered twice per week. In some embodiments, the protein is administered every other week. In some embodiments, the protein is administered every three weeks.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is schematic representation of an exemplary MSLN targeting trispecific antigen-binding protein where the protein has an constant core element comprising an anti-CD3c single chain variable fragment (scFv) and an anti-ALB variable heavy chain region; and an anti-MSLN binding domain that can be a VHH, a VH, scFv, a non-Ig binder, or a ligand.

FIG. 2 illustrates the effectivity of exemplary TriTAC molecules (2A2 and 2A4) in killing of OVCAR8 cells that expresses the target protein MSLN.

FIG. 3 illustrates that an exemplary TriTAC molecule of this disclosure (MH6T TriTAC) was able to direct T cells from five donors (donor 02; donor 86; donor 41; donor 81; and donor 35) to kill Caov3 cells. The figure also illustrates that a control TriTAC molecule (GFP TriTAC) was not able to direct T cells from the five donors (donor 02; donor 86; donor 41; donor 81; and donor 35) to kill Caov3 cells.

FIG. 4 illustrates that an exemplary TriTAC molecule of this disclosure (MH6T TriTAC) was able to direct T cells from five donors (donor 02; donor 86; donor 41; donor 81; and donor 35) to kill OVCAR3 cells. The figure also illustrates that a control TriTAC molecule (GFP TriTAC) was not able to direct T cells from the five donors (donor 02; donor 86; donor 41; donor 81; and donor 35) to kill OVCAR3 cells.

FIG. 5 illustrates that an exemplary TriTAC molecule of this disclosure (MH6T TriTAC) was able to direct T cells from a healthy donor to kill cells that express MSLN (OVCAR3 cells; Caov4 cells; OVCAR3 cells; and OVCAR8 cells). The figure also illustrates that the MH6T TriTAC was not able to direct T cells from the healthy donor to kill cells that do not express MSLN (MDAPCa2b cells; and NCI-H510A cells).

FIG. 6 illustrates that an exemplary TriTAC molecule of this disclosure (MH6T TriTAC) was able to direct T cells from cynomolgus monkeys to kill human ovarian cancer cell line (OVCAR3 cells; Caov3 cells). The figure also illustrates that a control TriTAC molecule (GFP TriTAC) was not able to direct the T cells from cynomolgus monkeys to kill human ovarian cancer cells lines (OVCAR3 cells; Caov3 cells).

FIG. 7 illustrates that an exemplary TriTAC molecule of this disclosure (MH6T TriTAC) was able to direct killing of MSLN expressing NCI-H₂₀₅₂ mesothelioma cells by T cells, in the presence or absence of human serum albumin (HSA).

FIG. 8 illustrates that an exemplary TriTAC molecule of this disclosure (MH6T TriTAC) was able to activate T cells from four healthy donors (donor 2; donor 86; donor 35; and donor 81), as demonstrated by secretion of TNF-α from the T cells, in presence of the MH6T TriTAC and MSLN-expressing Caov4 cells.

FIG. 9 illustrates that an exemplary TriTAC molecule of this disclosure (MH6T TriTAC) was able to activate T cells from four healthy donors (donor 2; donor 86; donor 35; and donor 81), as demonstrated by activation of CD69 expression on the T cells, in presence of the MH6T TriTAC and MSLN-expressing OVCAR8 cells.

FIG. 10A and FIG. 10B illustrate binding of an exemplary TriTAC molecule of this disclosure (MH6T TriTAC) to MSLN expressing cell lines or MSLN non-expressing cell lines. FIG. 10A shows binding of the MH6T TriTAC with MSLN expressing cells (Caov3 cells-top left panel, Caov4 cells-top right panel, OVCAR3 cells-bottom left panel, OVCAR8 cells-bottom right panel), and further illustrates lack of binding of a control TriTAC (GFP TriTAC) to the same cell lines. FIG. 10B shows lack of binding of both the MH6T TriTAC and the GFP TriTAC to MSLN non-expressing cell lines (MDCA2b cells-left panel, NCI-H510A cells-right panel).

FIG. 11 illustrates binding of an exemplary TriTAC molecule of this disclosure (MH6T TriTAC) to T cells from four healthy donors (donor 2-top left panel; donor 35-top right panel; donor 41-bottom left panel; donor 81-bottom right panel).

FIG. 12 illustrates that an exemplary TriTAC molecule of this disclosure (MH6T TriTAC) was able to inhibit tumor growth in NCG mice implanted with MSLN expressing NCI-H₂₉₂ cells.

FIG. 13 illustrates pharmacokinetic profile of an exemplary TriTAC molecule of this disclosure, MH6T TriTAC. Serum levels of the MH6T TriTAC molecule, at various time points following injection into two cynomolgus monkeys, are shown in the plot.

FIG. 14 shows results from binding affinity measurements of two exemplary trispecific molecules of this disclosure, TriTAC 74 and TriTAC 75, and EC₅₀ values for killing of SKOV3 and OVCAR cells by the two TriTAC molecules.

FIG. 15 illustrates pharmacokinetic profiles of two exemplary TriTAC molecules of this disclosure, TriTAC 75 and TriTAC 74. Serum levels of the TriTAC molecules, at various time points following injection into cynomolgus monkeys, are shown in the plot.

### DETAILED DESCRIPTION OF THE INVENTION

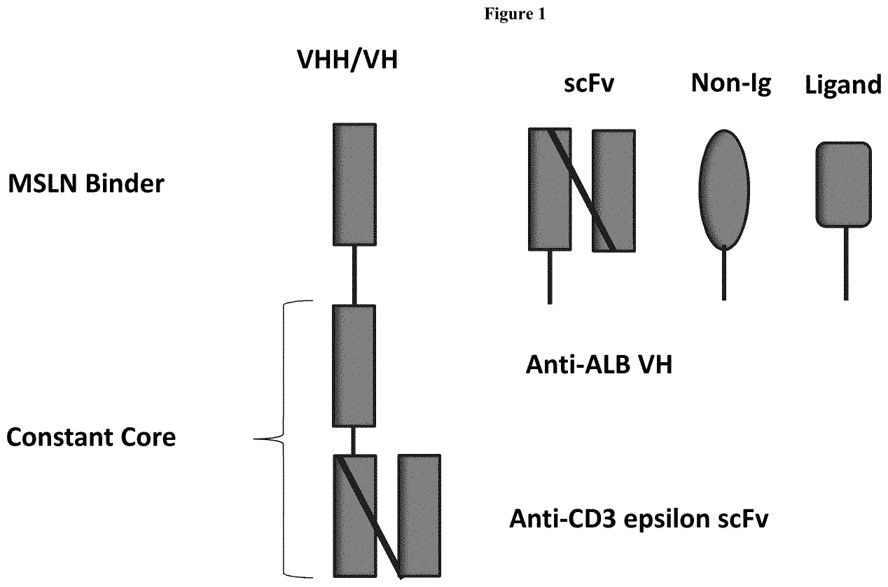

Described herein are trispecific proteins that target mesothelin (MSLN), pharmaceutical compositions thereof, as well as nucleic acids, recombinant expression vectors and host cells for making such proteins thereof. Also provided are methods of using the disclosed MSLN targeting trispecific proteins in the prevention, and/or treatment of diseases, conditions and disorders. The MSLN targeting trispecific proteins are capable of specifically binding to MSLN as well as CD3 and have a half-life extension domain, such as a domain binding to human albumin (ALB). FIG. 1 depicts one non-limiting example of a trispecific MSLN-binding protein.

In one aspect, the MSLN targeting trispecific proteins comprise a domain (A) which specifically binds to CD3, a domain (B) which specifically binds to human albumin (ALB), and a domain (C) which specifically binds to MSLN. The three domains in MSLN targeting trispecific proteins are arranged in any order. Thus, it is contemplated that the domain order of the MSLN targeting trispecific proteins are:
H₂N-(A)-(B)—(C)—COOH,
H₂N-(A)-(C)—(B)—COOH,
H₂N—(B)-(A)-(C)—COOH,
H₂N—(B)—(C)-(A)-COOH,
H₂N—(C)—(B)-(A)-COOH, or
H₂N—(C)-(A)-(B)—COOH.

In some embodiments, the MSLN targeting trispecific proteins have a domain order of H₂N-(A)-(B)—(C)—COOH. In some embodiments, the MSLN targeting trispecific proteins have a domain order of H₂N-(A)-(C)—(B)—COOH. In some embodiments, the MSLN targeting trispecific proteins have a domain order of H₂N—(B)-(A)-(C)—COOH. In some embodiments, the MSLN targeting trispecific proteins have a domain order of H₂N—(B)—(C)-(A)-COOH. In some embodiments, the MSLN targeting trispecific proteins have a domain order of H₂N—(C)—(B)-(A)-COOH. In some embodiments, the MSLN targeting trispecific proteins have a domain order of H₂N—(C)-(A)-(B)—COOH.

In some embodiments, the MSLN targeting trispecific proteins have the HSA binding domain as the middle domain, such that the domain order is H₂N-(A)-(B)—(C)—COOH or H₂N—(C)—(B)-(A)-COOH. It is contemplated that in such embodiments where the ALB binding domain as the middle domain, the CD3 and MSLN binding domains are afforded additional flexibility to bind to their respective targets.

In some embodiments, the MSLN targeting trispecific proteins described herein comprise a polypeptide having a sequence described in Table 1 (SEQ ID NO: 58-86, 98, 100, and 101) and subsequences thereof. In some embodiments, the trispecific antigen binding protein comprises a polypeptide having at least 70%-95% or more homology to a sequence described in Table 1 (SEQ ID NO: 58-86, 98, 100 and 101). In some embodiments, the trispecific antigen binding protein comprises a polypeptide having at least 70%, 75%, 80%, 85%, 90%, 95%, or more homology to a sequence described in Table 1 (SEQ ID NO: 58-86, 98, 100 and 101).

The MSLN targeting trispecific proteins described herein are designed to allow specific targeting of cells expressing MSLN by recruiting cytotoxic T cells. This improves efficacy compared to ADCC (antibody dependent cell-mediated cytotoxicity), which is using full length antibodies directed to a sole antigen and is not capable of directly recruiting cytotoxic T cells. In contrast, by engaging CD3 molecules expressed specifically on these cells, the MSLN targeting trispecific proteins can crosslink cytotoxic T cells with cells expressing MSLN in a highly specific fashion, thereby directing the cytotoxic potential of the T cell towards the target cell. The MSLN targeting trispecific proteins described herein engage cytotoxic T cells via binding to the surface-expressed CD3 proteins, which form part of the TCR. Simultaneous binding of several MSLN trispecific antigen-binding protein to CD3 and to MSLN expressed on the surface of particular cells causes T cell activation and mediates the subsequent lysis of the particular MSLN expressing cell. Thus, MSLN targeting trispecific proteins are contemplated to display strong, specific and efficient target cell killing. In some embodiments, the MSLN targeting trispecific proteins described herein stimulate target cell killing by cytotoxic T cells to eliminate pathogenic cells (e.g., tumor cells expressing MSLN). In some of such embodiments, cells are eliminated selectively, thereby reducing the potential for toxic side effects.

The MSLN targeting trispecific proteins described herein confer further therapeutic advantages over traditional monoclonal antibodies and other smaller bispecific molecules. Generally, the effectiveness of recombinant protein pharmaceuticals depends heavily on the intrinsic pharmacokinetics of the protein itself. One such benefit here is that the MSLN targeting trispecific proteins described herein have extended pharmacokinetic elimination half-time due to having a half-life extension domain such as a domain specific to HSA. In this respect, the MSLN targeting trispecific proteins described herein have an extended serum elimination half-time of about two, three, about five, about seven, about 10, about 12, or about 14 days in some embodiments. This contrasts to other binding proteins such as BiTE or DART molecules which have relatively much shorter elimination half-times. For example, the BiTE CD19×CD3 bispecific scFv-scFv fusion molecule requires continuous intravenous infusion (i.v.) drug delivery due to its short elimination half-time. The longer intrinsic half-times of the MSLN targeting trispecific proteins solve this issue thereby allowing for increased therapeutic potential such as low-dose pharmaceutical formulations, decreased periodic administration and/or novel pharmaceutical compositions.

The MSLN targeting trispecific proteins described herein also have an optimal size for enhanced tissue penetration and tissue distribution. Larger sizes limit or prevent penetration or distribution of the protein in the target tissues. The MSLN targeting trispecific proteins described herein avoid this by having a small size that allows enhanced tissue penetration and distribution. Accordingly, the MSLN targeting trispecific proteins described herein, in some embodiments have a size of about 50 kD to about 80 kD, about 50 kD to about 75 kD, about 50 kD to about 70 kD, or about 50 kD to about 65 kD. Thus, the size of the MSLN targeting trispecific proteins is advantageous over IgG antibodies which are about 150 kD and the BiTE and DART diabody molecules which are about 55 kD but are not half-life extended and therefore cleared quickly through the kidney.

In further embodiments, the MSLN targeting trispecific proteins described herein have an optimal size for enhanced tissue penetration and distribution. In these embodiments, the MSLN targeting trispecific proteins are constructed to be as small as possible, while retaining specificity toward its targets. Accordingly, in these embodiments, the MSLN targeting trispecific proteins described herein have a size of about 20 kD to about 40 kD or about 25 kD to about 35 kD to about 40 kD, to about 45 kD, to about 50 kD, to about 55 kD, to about 60 kD, to about 65 kD. In some embodiments, the MSLN targeting trispecific proteins described herein have a size of about 50kD, 49, kD, 48 kD, 47 kD, 46 kD, 45 kD, 44 kD, 43 kD, 42 kD, 41 kD, 40 kD, about 39 kD, about 38 kD, about 37 kD, about 36 kD, about 35 kD, about 34 kD, about 33 kD, about 32 kD, about 31 kD, about 30 kD, about 29 kD, about 28 kD, about 27 kD, about 26 kD, about 25 kD, about 24 kD, about 23 kD, about 22 kD, about 21 kD, or about 20 kD. An exemplary approach to the small size is through the use of single domain antibody (sdAb) fragments for each of the domains. For example, a particular MSLN trispecific antigen-binding protein has an anti-CD3 sdAb, anti-ALB sdAb and an sdAb for MSLN. This reduces the size of the exemplary MSLN trispecific antigen-binding protein to under 60 kD. Thus in some embodiments, the domains of the MSLN targeting trispecific proteins are all single domain antibody (sdAb) fragments. In other embodiments, the MSLN targeting trispecific proteins described herein comprise small molecule entity (SME) binders for ALB and/or the MSLN. SME binders are small molecules averaging about 500 to 2000 Da in size and are attached to the MSLN targeting trispecific proteins by known methods, such as sortase ligation or conjugation. In these instances, one of the domains of MSLN trispecific antigen-binding protein is a sortase recognition sequence, e.g., LPETG (SEQ ID NO: 97). To attach a SME binder to MSLN trispecific antigen-binding protein with a sortase recognition sequence, the protein is incubated with a sortase and a SME binder whereby the sortase attaches the SME binder to the recognition sequence. Known SME binders include MIP-1072 and MIP-1095 which bind to mesothelin. In yet other embodiments, the domain which binds to MSLN of MSLN targeting trispecific proteins described herein comprise a knottin peptide for binding MSLN. Knottins are disufide-stabilized peptides with a cysteine knot scaffold and have average sizes about 3.5 kD. Knottins have been contemplated for binding to certain tumor molecules such as MSLN. In further embodiments, domain which binds to MSLN of MSLN targeting trispecific proteins described herein comprise a natural MSLN ligand.

Another feature of the MSLN targeting trispecific proteins described herein is that they are of a single-polypeptide design with flexible linkage of their domains. This allows for facile production and manufacturing of the MSLN targeting trispecific proteins as they can be encoded by single cDNA molecule to be easily incorporated into a vector. Further, because the MSLN targeting trispecific proteins described herein are a monomeric single polypeptide chain, there are no chain pairing issues or a requirement for dimerization. It is contemplated that the MSLN targeting trispecific proteins described herein have a reduced tendency to aggregate unlike other reported molecules such as bispecific proteins with Fc-gamma immunoglobulin domains.

In the MSLN targeting trispecific proteins described herein, the domains are linked by internal linkers L1 and L2, where L1 links the first and second domain of the MSLN targeting trispecific proteins and L2 links the second and third domains of the MSLN targeting trispecific proteins. Linkers L1 and L2 have an optimized length and/or amino acid composition. In some embodiments, linkers L1 and L2 are the same length and amino acid composition. In other embodiments, L1 and L2 are different. In certain embodiments, internal linkers L1 and/or L2 are “short”, i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, internal linkers L1 and/or L2 are “long”, i.e., consist of 15, 20 or 25 amino acid residues. In some embodiments, these internal linkers consist of about 3 to about 15, for example 8, 9 or 10 contiguous amino acid residues. Regarding the amino acid composition of the internal linkers L1 and L2, peptides are selected with properties that confer flexibility to the MSLN targeting trispecific proteins, do not interfere with the binding domains as well as resist cleavage from proteases. For example, glycine and serine residues generally provide protease resistance. Examples of internal linkers suitable for linking the domains in the MSLN targeting trispecific proteins include but are not limited to (GS). (SEQ ID NO: 87), (GGS)ₙ (SEQ ID NO: 88), (GGGS)ₙ (SEQ ID NO: 89), (GGSG)ₙ (SEQ ID NO: 90), (GGSGG)ₙ (SEQ ID NO: 91), (GGGGS)ₙ (SEQ ID NO: 92), (GGGGG)ₙ (SEQ ID NO: 93), or (GGG)ₙ (SEQ ID NO: 94), wherein n is 1, 2, 3, 4, 5, 6, 7, 8, 9, or 10. In one embodiment, internal linker L1 and/or L2 is (GGGGS)₄ (SEQ ID NO: 95) or (GGGGS)₃ (SEQ ID NO: 96).

### CD3 Binding Domain

The specificity of the response of T cells is mediated by the recognition of antigen (displayed in context of a major histocompatibility complex, MEW) by the TCR. As part of the TCR, CD3 is a protein complex that includes a CD3γ (gamma) chain, a CD3δ (delta) chain, and two CD3ε (epsilon) chains which are present on the cell surface. CD3 associates with the α (alpha) and β (beta) chains of the TCR as well as CD3 ζ (zeta) altogether to comprise the complete TCR. Clustering of CD3 on T cells, such as by immobilized anti-CD3 antibodies leads to T cell activation similar to the engagement of the T cell receptor but independent of its clone-typical specificity.

In one aspect, the MSLN targeting trispecific proteins described herein comprise a domain which specifically binds to CD3. In one aspect, the MSLN targeting trispecific proteins described herein comprise a domain which specifically binds to human CD3. In some embodiments, the MSLN targeting trispecific proteins described herein comprise a domain which specifically binds to CD3γ. In some embodiments, the MSLN targeting trispecific proteins described herein comprise a domain which specifically binds to CD3δ. In some embodiments, the MSLN targeting trispecific proteins described herein comprise a domain which specifically binds to CD3ε.

In further embodiments, the MSLN targeting trispecific proteins described herein comprise a domain which specifically binds to the TCR. In certain instances, the MSLN targeting trispecific proteins described herein comprise a domain which specifically binds the a chain of the TCR. In certain instances, the MSLN targeting trispecific proteins described herein comprise a domain which specifically binds the β chain of the TCR.

In certain embodiments, the CD3 binding domain of the MSLN targeting trispecific proteins described herein exhibit not only potent CD3 binding affinities with human CD3, but show also excellent crossreactivity with the respective cynomolgus monkey CD3 proteins. In some instances, the CD3 binding domain of the MSLN targeting trispecific proteins are cross-reactive with CD3 from cynomolgus monkey. In certain instances, human:cynomolgous K_{D} ratios for CD3 are between 5 and 0.2.

In some embodiments, the CD3 binding domain of the MSLN trispecific antigen-binding protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the MSLN trispecific antigen-binding protein will ultimately be used in. For example, for use in humans, it may be beneficial for the CD3 binding domain of the MSLN trispecific antigen-binding protein to comprise human or humanized residues from the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-CD3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD3 binding domain described herein, e.g., a humanized or human anti-CD3 binding domain comprising one or more, e.g., all three, LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lamda) light chain framework. In other instances, the light chain framework region is a κ (kappa) light chain framework.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In certain instances, the complementary determining regions of the heavy chain and/or the light chain are derived from known anti-CD3 antibodies, such as, for example, muromonab-CD3 (OKT3), otelixizumab (TRX4), teplizumab (MGA031), visilizumab (Nuvion), SP34, TR-66 or X35-3, VIT3, BMA030 (BW264/56), CLB-T3/3, CRIS7, YTH_{12.5,} F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In one embodiment, the anti-CD3 binding domain is a single chain variable fragment (scFv) comprising a light chain and a heavy chain of an amino acid sequence provided herein. As used herein, “single chain variable fragment” or “scFv” refers to an antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived. In an embodiment, the anti-CD3 binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region-scFv linker-heavy chain variable region or heavy chain variable region-scFv linker-light chain variable region.

In some instances, scFvs which bind to CD3 are prepared according to known methods. For example, scFv molecules can be produced by linking VH and VL regions together using flexible polypeptide linkers. The scFv molecules comprise a scFv linker (e.g., a Ser-Gly linker) with an optimized length and/or amino acid composition. Accordingly, in some embodiments, the length of the scFv linker is such that the VH or VL domain can associate intermolecularly with the other variable domain to form the CD3 binding site. In certain embodiments, such scFv linkers are “short”, i.e. consist of 0, 1, 2, 3, 4, 5, 6, 7, 8, 9, 10, 11 or 12 amino acid residues. Thus, in certain instances, the scFv linkers consist of about 12 or less amino acid residues. In the case of 0 amino acid residues, the scFv linker is a peptide bond. In some embodiments, these scFv linkers consist of about 3 to about 15, for example 8, 9 or 10 contiguous amino acid residues. Regarding the amino acid composition of the scFv linkers, peptides are selected that confer flexibility, do not interfere with the variable domains as well as allow inter-chain folding to bring the two variable domains together to form a functional CD3 binding site. For example, scFv linkers comprising glycine and serine residues generally provide protease resistance. In some embodiments, linkers in a scFv comprise glycine and serine residues. The amino acid sequence of the scFv linkers can be optimized, for example, by phage-display methods to improve the CD3 binding and production yield of the scFv. Examples of peptide scFv linkers suitable for linking a variable light domain and a variable heavy domain in a scFv include but are not limited to (GS)ₙ (SEQ ID NO: 87), (GGS)n (SEQ ID NO: 88), (GGGS)ₙ (SEQ ID NO: 89), (GGSG)ₙ (SEQ ID NO: 90), (GGSGG)ₙ (SEQ ID NO: 91), (GGGGS)ₙ (SEQ ID NO: 92), (GGGGG)ₙ (SEQ ID NO: 93), or (GGG)ₙ (SEQ ID NO: 94), wherein n is 1, 2, 3, 4, 5, 6, 7, 8, 9, or 10. In one embodiment, the scFv linker can be (GGGGS)4 (SEQ ID NO: 95) or (GGGGS)3 (SEQ ID NO: 96). Variation in the linker length may retain or enhance activity, giving rise to superior efficacy in activity studies.

In some embodiments, CD3 binding domain of MSLN trispecific antigen-binding protein has an affinity to CD3 on CD3 expressing cells with a K_{D} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In some embodiments, the CD3 binding domain of MSLN trispecific antigen-binding protein has an affinity to CD3ε, γ, or δ with a K_{D} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In further embodiments, CD3 binding domain of MSLN trispecific antigen-binding protein has low affinity to CD3, i.e., about 100 nM or greater.

The affinity to bind to CD3 can be determined, for example, by the ability of the MSLN trispecific antigen-binding protein itself or its CD3 binding domain to bind to CD3 coated on an assay plate; displayed on a microbial cell surface; in solution; etc. The binding activity of the MSLN trispecific antigen-binding protein itself or its CD3 binding domain of the present disclosure to CD3 can be assayed by immobilizing the ligand (e.g., CD3) or the MSLN trispecific antigen-binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

### Half-Life Extension Domain

Contemplated herein are domains which extend the half-life of an antigen-binding domain. Such domains are contemplated to include but are not limited to Albumin binding domains, Fc domains, small molecules, and other half-life extension domains known in the art.

Human albumin (ALB) (molecular mass ˜67 kDa) is the most abundant protein in plasma, present at about 50 mg/ml (600 μM), and has a half-life of around 20 days in humans. ALB serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in an in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the MSLN targeting trispecific proteins described herein comprise a half-life extension domain, for example a domain which specifically binds to ALB. In some embodiments, the ALB binding domain of MSLN trispecific antigen-binding protein can be any domain that binds to ALB including but not limited to domains from a monoclonal antibody, a polyclonal antibody, a recombinant antibody, a human antibody, a humanized antibody. In some embodiments, the ALB binding domain is a single chain variable fragments (scFv), single-domain antibody such as a heavy chain variable domain (VH), a light chain variable domain (VL) and a variable domain (VHH) of camelid derived single domain antibody, peptide, ligand or small molecule entity specific for HSA. In certain embodiments, the ALB binding domain is a single-domain antibody. In other embodiments, the HSA binding domain is a peptide. In further embodiments, the HSA binding domain is a small molecule. It is contemplated that the HSA binding domain of MSLN trispecific antigen-binding protein is fairly small and no more than 25 kD, no more than 20 kD, no more than 15 kD, or no more than 10 kD in some embodiments. In certain instances, the ALB binding is 5 kD or less if it is a peptide or small molecule entity.

The half-life extension domain of MSLN trispecific antigen-binding protein provides for altered pharmacodynamics and pharmacokinetics of the MSLN trispecific antigen-binding protein itself. As above, the half-life extension domain extends the elimination half-time. The half-life extension domain also alters pharmacodynamic properties including alteration of tissue distribution, penetration, and diffusion of the trispecific antigen-binding protein. In some embodiments, the half-life extension domain provides for improved tissue (including tumor) targeting, tissue distribution, tissue penetration, diffusion within the tissue, and enhanced efficacy as compared with a protein without an half-life extension domain. In one embodiment, therapeutic methods effectively and efficiently utilize a reduced amount of the trispecific antigen-binding protein, resulting in reduced side effects, such as reduced non-tumor cell cytotoxicity.

Further, the binding affinity of the half-life extension domain can be selected so as to target a specific elimination half-time in a particular trispecific antigen-binding protein. Thus, in some embodiments, the half-life extension domain has a high binding affinity. In other embodiments, the half-life extension domain has a medium binding affinity. In yet other embodiments, the half-life extension domain has a low or marginal binding affinity. Exemplary binding affinities include KD concentrations at 10 nM or less (high), between 10 nM and 100 nM (medium), and greater than 100 nM (low). As above, binding affinities to ALB are determined by known methods such as Surface Plasmon Resonance (SPR).

In some embodiments, ALB binding domains described herein comprise a single domain antibody.

### Mesothelin (MSLN) Binding Domain

Mesothelin is a glycoprotein present on the surface of cells of the mesothelial lining of the peritoneal, pleural and pericardial body cavities. The mesothelin gene (MSLN) encodes a 71 kD precursor protein that is processed to a 40 kD protein termed mesothelin, which is a glycosyl-phosphatidylinositol-anchored glycoprotein present on the cell surface (Chang, et al, Proc Natl Acad Sci USA (1996) 93:136-40). The mesothelin cDNA was cloned from a library prepared from the HPC-Y5 cell line (Kojima et al. (1995) J. Biol. Chem. 270:21984-21990). The cDNA also was cloned using the monoclonal antibody K1, which recognizes mesotheliomas (Chang and Pastan (1996) Proc. Natl. Acad. Sci. USA 93:136-40). Mesothelin is a differentiation antigen whose expression in normal human tissues is limited to mesothelial cells lining the body cavity, such as the pleura, pericardium and peritoneum. Mesothelin is also highly expressed in several different human cancers, including mesotheliomas, pancreatic adenocarcinomas, ovarian cancers, stomach and lung adenocarcinomas. (Hassan, et al., Eur J Cancer (2008) 44:46-53) (Ordonez, Am J Surg Pathol (2003) 27:1418-28; Ho, et al., Clin Cancer Res (2007) 13:1571-5). Mesothelin is overexpressed in a vast majority of primary pancreatic adenocarcinomas with rare and weak expression seen in benign pancreatic tissue. Argani P, et al. Clin Cancer Res. 2001; 7(12):3862-3868. Epithelial malignant pleural mesothelioma (MPM) universally expresses mesothelin while sarcomatoid MPM likely does not express mesothelin. Most serous epithelial ovarian carcinomas, and the related primary peritoneal carcinomas, express mesothelin.

Mesothelin can also be used a marker for diagnosis and prognosis of certain types of cancer because trace amounts of mesothelin can be detected in the blood of some patients with mesothelin-positive cancers (Cristaudo et al., Clin. Cancer Res. 13:5076-5081, 2007). It has been reported that mesothelin may be released into serum through deletion at its carboxyl terminus or by proteolytic cleavage from its membrane bound form (Hassan et al., Clin. Cancer Res. 10:3937-3942, 2004). An increase in the soluble form of mesothelin was detectable several years before malignant mesotheliomas occurred among workers exposed to asbestos (Creaney and Robinson, Hematol. Oncol. Clin. North Am. 19:1025-1040, 2005). Furthermore, patients with ovarian, pancreatic, and lung cancers also have elevated soluble mesothelin in serum (Cristaudo et al., Clin. Cancer Res. 13:5076-5081, 2007; Hassan et al., Clin. Cancer Res. 12:447-453, 2006; Croso et al., Cancer Detect. Prey. 30:180-187, 2006). Accordingly, mesothelin is an appropriate target for methods of disease prevention or treatment and there is a need for effective antibodies specific for mesothelin.

It has been shown that cell surface mature mesothelin comprises three distinct domains, namely Regions I (comprising residues 296-390), II (comprising residues 391-486), and III (comprising residue 487-598). (Tang et al., A human single-domain antibody elicits potent antitumor activity by targeting an epitope in mesothelin close to the cancer cell surface, Mol. Can. Therapeutics, 12(4): 416-426, 2013). The first antibodies generated against mesothelin for therapeutic intervention were designed to interfere with the interaction between mesothelin and CA-125. Phage display identified the Fv SS, which was affinity optimized and used to generate a recombinant immunotoxin targeting mesothelin, SS1P. The MORAb-009 antibody amatuximab, which also uses SS1, recognizes a non-linear epitope in the amino terminal 64 amino acids of mesothelin, within region I. The SS1 Fv was also used to generate chimeric antigen receptor-engineered T cells. Recently, new anti-mesothelin antibodies have been reported that recognize other regions of the mesothelin protein.

There is still a need for having available further options for the treatment of solid tumor diseases related to the overexpression of mesothelin, such as ovarian cancer, pancreatic cancer, mesothelioma, lung cancer, gastric cancer and triple negative breast cancer. The present disclosure provides, in certain embodiments, MSLN targeting trispecific proteins containing binding domains which specifically bind to MSLN on the surface of tumor target cells.

The design of the MSLN targeting trispecific proteins described herein allows the binding domain to MSLN to be flexible in that the binding domain to MSLN can be any type of binding domain, including but not limited to, domains from a monoclonal antibody, a polyclonal antibody, a recombinant antibody, a human antibody, a humanized antibody. In some embodiments, the binding domain to MSLN is a single chain variable fragments (scFv), single-domain antibody such as a heavy chain variable domain (VH), a light chain variable domain (VL) and a variable domain (VHH) of camelid derived single domain antibody. In other embodiments, the binding domain to MSLN is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to MSLN is a ligand or peptide that binds to or associates with MSLN. In yet further embodiments, the binding domain to MSLN is a knottin. In yet further embodiments, the binding domain to MSLN is a small molecular entity.

In some embodiments, the MSLN binding domain binds to a protein comprising the sequence of SEQ ID NO: 57. In some embodiments, the MSLN binding domain binds to a protein comprising a truncated sequence compared to SEQ ID NO: 57.

In some embodiments, the MSLN binding domains disclosed herein recognize full-length mesothelin. In certain instances, the MSLN binding domains disclosed herein recognize an epitope in region I (comprising amino acid residues 296-390 of SEQ ID NO: 57), region II (comprising amino acid residue 391-486 of SEQ ID NO: 57), or region III (comprising amino acid residues 487-598 of SEQ ID NO: 57) of mesothelin. It is contemplated that the MSLN binding domains of the present disclosure may, in some embodiments, recognize and bind to epitopes that are located outside regions I, II, or III of mesothelin. In yet other embodiments are disclosed MSLN binding domains that recognize and bind to an epitope different than the MORAb-009 antibody.

In some embodiments, the MSLN binding domain is an anti-MSLN antibody or an antibody variant. As used herein, the term “antibody variant” refers to variants and derivatives of an antibody described herein. In certain embodiments, amino acid sequence variants of the anti-MSLN antibodies described herein are contemplated. For example, in certain embodiments amino acid sequence variants of anti-MSLN antibodies described herein are contemplated to improve the binding affinity and/or other biological properties of the antibodies. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, e.g., antigen-binding. In certain embodiments, antibody variants having one or more amino acid substitutions are provided. Sites of interest for substitution mutagenesis include the CDRs and framework regions. Examples of such substitutions are described below. Amino acid substitutions may be introduced into an antibody of interest and the products screened for a desired activity, e.g., retained/improved antigen binding, decreased immunogenicity, or improved T-cell mediated cytotoxicity (TDCC). Both conservative and non-conservative amino acid substitutions are contemplated for preparing the antibody variants.

In another example of a substitution to create a variant anti-MSLN antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding.

In some embodiments, the MSLN binding domain of the MSLN targeting trispecific protein is a single domain antibody such as a heavy chain variable domain (VH), a variable domain (VHH) of a llama derived sdAb, a peptide, a ligand or a small molecule entity specific for mesothelin. In some embodiments, the mesothelin binding domain of the MSLN targeting trispecific protein described herein is any domain that binds to mesothelin including but not limited to domains from a monoclonal antibody, a polyclonal antibody, a recombinant antibody, a human antibody, a humanized antibody. In certain embodiments, the MSLN binding domain is a single-domain antibody. In other embodiments, the MSLN binding domain is a peptide. In further embodiments, the MSLN binding domain is a small molecule.

Generally, it should be noted that the term single domain antibody as used herein in its broadest sense is not limited to a specific biological source or to a specific method of preparation. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. For example, in some embodiments, the single domain antibodies of the disclosure are obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, and in particular from a species of mammal, such as from a human being, or by expression of a nucleic acid encoding such a camelized VH domain; (5) by “camelisation” of a “domain antibody” or “Dab”, or by expression of a nucleic acid encoding such a camelized VH domain; (6) by using synthetic or semi-synthetic techniques for preparing proteins, polypeptides or other amino acid sequences; (7) by preparing a nucleic acid encoding a single domain antibody using techniques for nucleic acid synthesis known in the field, followed by expression of the nucleic acid thus obtained; and/or (8) by any combination of one or more of the foregoing.

In one embodiment, a single domain antibody corresponds to the VHH domains of naturally occurring heavy chain antibodies directed against MSLN. As further described herein, such VHH sequences can generally be generated or obtained by suitably immunizing a species of Llama with MSLN, (i.e., so as to raise an immune response and/or heavy chain antibodies directed against MSLN), by obtaining a suitable biological sample from said Llama (such as a blood sample, serum sample or sample of B-cells), and by generating VHH sequences directed against MSLN, starting from said sample, using any suitable technique known in the field.

In another embodiment, such naturally occurring VHH domains against MSLN, are obtained from naive libraries of Camelid VHH sequences, for example by screening such a library using MSLN, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from naive VHH libraries are used, such as VHH libraries obtained from naive VHH libraries by techniques such as random mutagenesis and/or CDR shuffling, as for example described in WO 00/43507.

In a further embodiment, yet another technique for obtaining VHH sequences directed against MSLN, involves suitably immunizing a transgenic mammal that is capable of expressing heavy chain antibodies (i.e., so as to raise an immune response and/or heavy chain antibodies directed against MSLN), obtaining a suitable biological sample from said transgenic mammal (such as a blood sample, serum sample or sample of B-cells), and then generating VHH sequences directed against MSLN, starting from said sample, using any suitable technique known in the field. For example, for this purpose, the heavy chain antibody-expressing rats or mice and the further methods and techniques described in WO 02/085945 and in WO 04/049794 can be used.

In some embodiments, an anti-MSLN single domain antibody of the MSLN targeting trispecific protein comprises a single domain antibody with an amino acid sequence that corresponds to the amino acid sequence of a naturally occurring VHH domain, but that has been “humanized”, i.e., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above). This can be performed in a manner known in the field, which will be clear to the skilled person, for example on the basis of the further description herein. Again, it should be noted that such humanized anti-MSLN single domain antibodies of the disclosure are obtained in any suitable manner known per se (i.e., as indicated under points (1)-(8) above) and thus are not strictly limited to polypeptides that have been obtained using a polypeptide that comprises a naturally occurring VHH domain as a starting material. In some additional embodiments, a single domain anti-MSLN antibody, as described herein, comprises a single domain antibody with an amino acid sequence that corresponds to the amino acid sequence of a naturally occurring VH domain, but that has been “camelized”, i.e., by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. Such “camelizing” substitutions are preferably inserted at amino acid positions that form and/or are present at the VH-VL interface, and/or at the so-called Camelidae hallmark residues (see for example WO 94/04678 and Davies and Riechmann (1994 and 1996)). Preferably, the VH sequence that is used as a starting material or starting point for generating or designing the camelized single domain is preferably a VH sequence from a mammal, more preferably the VH sequence of a human being, such as a VH3 sequence. However, it should be noted that such camelized anti-MSLN single domain antibodies of the disclosure, in certain embodiments, are obtained in any suitable manner known in the field (i.e., as indicated under points (1)-(8) above) and thus are not strictly limited to polypeptides that have been obtained using a polypeptide that comprises a naturally occurring VH domain as a starting material. For example, as further described herein, both “humanization” and “camelization” is performed by providing a nucleotide sequence that encodes a naturally occurring VHH domain or VH domain, respectively, and then changing, one or more codons in said nucleotide sequence in such a way that the new nucleotide sequence encodes a “humanized” or “camelized” single domain antibody, respectively. This nucleic acid can then be expressed, so as to provide a desired anti-MSLN single domain antibody of the disclosure. Alternatively, in other embodiments, based on the amino acid sequence of a naturally occurring VHH domain or VH domain, respectively, the amino acid sequence of the desired humanized or camelized anti-MSLN single domain antibody of the disclosure, respectively, are designed and then synthesized de novo using known techniques for peptide synthesis. In some embodiments, based on the amino acid sequence or nucleotide sequence of a naturally occurring VHH domain or VH domain, respectively, a nucleotide sequence encoding the desired humanized or camelized anti-MSLN single domain antibody of the disclosure, respectively, is designed and then synthesized de novo using known techniques for nucleic acid synthesis, after which the nucleic acid thus obtained is expressed in using known expression techniques, so as to provide the desired anti-MSLN single domain antibody of the disclosure.

Other suitable methods and techniques for obtaining the anti-MSLN single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide an anti-MSLN single domain antibody of the disclosure or a nucleotide sequence or nucleic acid encoding the same.

In some embodiments, the MSLN binding domain is an anti-MSLN specific antibody comprising a heavy chain variable complementarity determining region CDR1, a heavy chain variable CDR2, a heavy chain variable CDR3, a light chain variable CDR1, a light chain variable CDR2, and a light chain variable CDR3. In some embodiments, the MSLN binding domain comprises any domain that binds to MSLN including but not limited to domains from a monoclonal antibody, a polyclonal antibody, a recombinant antibody, a human antibody, a humanized antibody, or antigen binding fragments such as single domain antibodies (sdAb), Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the MSLN binding domain is a single domain antibody. In some embodiments, the anti-MSLN single domain antibody comprises heavy chain variable complementarity determining regions (CDR), CDR1, CDR2, and CDR3.

In some embodiments, the MSLN binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the MSLN binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the MSLN binding domain comprises an amino acid sequence selected from SEQ ID NOs: 1-40, and 102-105. In some embodiments, the framework region 1 of a MSLN binding trispecific protein of this disclosure comprises a sequence as in any one of SEQ ID Nos.: 223-261. In some embodiments, the framework region 2 of a MSLN binding trispecific protein of this disclosure comprises a sequence as in any one of SEQ ID Nos.: 262-300. In some embodiments, the framework region 3 of a MSLN binding trispecific protein of this disclosure comprises a sequence as in any one of SEQ ID Nos.: 301-339. In some embodiments, the framework region 4 of a MSLN binding trispecific protein of this disclosure comprises a sequence as in any one of SEQ ID Nos.: 340-378.

In some embodiments, the CDR1 comprises the amino acid sequence as set forth in SEQ ID NO: 51 or a variant having one, two, three, four, five, six, seven, eight, nine, or ten amino acid substitutions in SEQ ID NO: 51. In some embodiments, the CDR2 comprises a sequence as set forth in SEQ ID NO: 52 or a variant having one, two, three, four, five, six, seven, eight, nine, or ten amino acid substitutions in SEQ ID NO: 52. In some embodiments, the CDR3 comprises a sequence as set forth in SEQ ID NO: 53 or a variant having one, two, three, four, five, six, seven, eight, nine, or ten amino acid substitutions in SEQ ID NO: 53.

In some embodiments, the CDR1 comprises the amino acid sequence as set forth in SEQ ID NO: 54 or a variant having one, two, three, four, five, six, seven, eight, nine, or ten amino acid substitutions in SEQ ID NO: 54. In some embodiments, the CDR2 comprises a sequence as set forth in SEQ ID NO: 55 or a variant having one, two, three, four, five, six, seven, eight, nine, or ten amino acid substitutions in SEQ ID NO: 55. In some embodiments, the CDR3 comprises a sequence as set forth in SEQ ID NO: 56 or a variant having one, two, three, four, five, six, seven, eight, nine, or ten amino acid substitutions in SEQ ID NO: 56.

In some embodiments, the CDR1 comprises the amino acid sequence as set forth in any one of SEQ ID Nos.: 106-144 or a variant having one, two, three, four, five, six, seven, eight, nine, or ten amino acid substitutions in any one of SEQ ID Nos.: 106-144. In some embodiments, the CDR2 comprises a sequence as set forth in any one of SEQ ID Nos.: 145-183 or a variant having one, two, three, four, five, six, seven, eight, nine, or ten amino acid substitutions in any one of SEQ ID Nos.: 145-183. In some embodiments, the CDR3 comprises a sequence as set forth in any one of SEQ ID Nos.: 184-222 or a variant having one, two, three, four, five, six, seven, eight, nine, or ten amino acid substitutions in any one of SEQ ID Nos.: 184-222.

The MSLN binding domains of the present disclosure, in certain examples, comprise one or more conserved regions. The conserved regions comprise sequences as set forth in SEQ ID NOs: 41-49, or variants comprising one or more amino acid residue substitutions relative to said sequences. Exemplary embodiments include MSLN binding proteins comprising one or more conserved regions selected from SEQ ID NOs: 41-44, or variants comprising one or more amino acid residue substitutions relative to said sequences. In some cases, the MSLN binding domain comprises (i) a stretch of amino acids corresponding to SEQ ID NO: 41, (ii) a stretch of amino acids corresponding to SEQ ID NO: 42, (iii) a stretch of amino acids corresponding to SEQ ID NO: 43, and (iv) a stretch of amino acids corresponding to SEQ ID NO: 44.

Further exemplary embodiments include MSLN binding domains comprising one or more conserved regions selected from SEQ ID NOs: 45-50, or variants comprising one or more amino acid residue substitutions relative to said sequences. In some cases, the MSLN binding domain comprises (i) a stretch of amino acids corresponding to SEQ ID NO: 45, (ii) a stretch of amino acids corresponding to SEQ ID NO: 46, (iii) a stretch of amino acids corresponding to SEQ ID NO: 47, (iv) a stretch of amino acids corresponding to SEQ ID NO: 48, (v) a stretch of amino acid corresponding to SEQ ID NO: 49, and (vi) a stretch of amino acids corresponding to SEQ ID NO: 50.

In various embodiments, the MSLN binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOs: 1-29.

In various embodiments, the MSLN binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOs: 30-40, and 102-105.

In various embodiments, a complementarity determining region of the MSLN binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 51, SEQ ID NO: 54, or any one of SEQ ID Nos.: 106-144.

In various embodiments, a complementarity determining region of the MSLN binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 52, SEQ ID NO: 55, or any one of SEQ ID Nos.: 145-183.

In various embodiments, a complementarity determining region of the MSLN binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 53, SEQ ID NO: 56, or any one of SEQ ID Nos.: 184-222.

In some embodiments, the MSLN binding protein, according to any one of the above embodiments, is a single domain antibody comprising the sequence of SEQ ID NO: 1. In some embodiments, the MSLN binding protein, according to any one of the above embodiments, is a single domain antibody comprising the sequence of SEQ ID NO: 2. In some embodiments, the MSLN binding protein, according to any one of the above embodiments, is a single domain antibody comprising the sequence of SEQ ID NO: 3. In some embodiments, the MSLN binding protein, according to any one of the above embodiments, is a single domain antibody comprising the sequence of SEQ ID NO: 4. In some embodiments, the MSLN binding protein, according to any one of the above embodiments, is a single domain antibody comprising the sequence of SEQ ID NO: 5. In some embodiments, the MSLN binding protein, according to any one of the above embodiments, is a single domain antibody comprising the sequence of SEQ ID NO: 6. In some embodiments, the MSLN binding protein, according to any one of the above embodiments, is a single domain antibody comprising the sequence of SEQ ID NO: 7. In some embodiments, the MSLN binding protein, according to any one of the above embodiments, is a single domain antibody comprising the sequence of SEQ ID NO: 8. In some embodiments, the MSLN binding protein, according to any one of the above embodiments, is a single domain antibody comprising the sequence of SEQ ID NO: 9. In some embodiments, the MSLN binding protein, according to any one of the above embodiments, is a single domain antibody comprising the sequence of SEQ ID NO: 10. In some embodiments, the MSLN binding protein, according to any one of the above embodiments, is a single domain antibody comprising the sequence of SEQ ID NO: 11. In some embodiments, the MSLN binding protein, according to any one of the above embodiments, is a single domain antibody comprising the sequence of SEQ ID NO: 12. In some embodiments, the MSLN binding protein, according to any one of the above embodiments, is a single domain antibody comprising the sequence of SEQ ID NO: 13. In some embodiments, the MSLN binding protein, according to any one of the above embodiments, is a single domain antibody comprising the sequence of SEQ ID NO: 14. In some embodiments, the MSLN binding protein, according to any one of the above embodiments, is a single domain antibody comprising the sequence of SEQ ID NO: 15. In some embodiments, the MSLN binding protein, according to any one of the above embodiments, is a single domain antibody comprising the sequence of SEQ ID NO: 16. In some embodiments, the MSLN binding protein, according to any one of the above embodiments, is a single domain antibody comprising the sequence of SEQ ID NO: 17. In some embodiments, the MSLN binding protein, according to any one of the above embodiments, is a single domain antibody comprising the sequence of SEQ ID NO: 18. In some embodiments, the MSLN binding protein, according to any one of the above embodiments, is a single domain antibody comprising the sequence of SEQ ID NO: 19. In some embodiments, the MSLN binding protein, according to any one of the above embodiments, is a single domain antibody comprising the sequence of SEQ ID NO: 20. In some embodiments, the MSLN binding protein, according to any one of the above embodiments, is a single domain antibody comprising the sequence of SEQ ID NO: 21. In some embodiments, the MSLN binding protein, according to any one of the above embodiments, is a single domain antibody comprising the sequence of SEQ ID NO: 22. In some embodiments, the MSLN binding protein, according to any one of the above embodiments, is a single domain antibody comprising the sequence of SEQ ID NO: 23. In some embodiments, the MSLN binding protein, according to any one of the above embodiments, is a single domain antibody comprising the sequence of SEQ ID NO: 24. In some embodiments, the MSLN binding protein, according to any one of the above embodiments, is a single domain antibody comprising the sequence of SEQ ID NO: 25. In some embodiments, the MSLN binding protein, according to any one of the above embodiments, is a single domain antibody comprising the sequence of SEQ ID NO: 26. In some embodiments, the MSLN binding protein, according to any one of the above embodiments, is a single domain antibody comprising the sequence of SEQ ID NO: 27. In some embodiments, the MSLN binding protein, according to any one of the above embodiments, is a single domain antibody comprising the sequence of SEQ ID NO: 28. In some embodiments, the MSLN binding protein, according to any one of the above embodiments, is a single domain antibody comprising the sequence of SEQ ID NO: 29.

In some embodiments, the MSLN binding protein, according to any one of the above embodiments, is a humanized single domain antibody comprising the sequence of SEQ ID NO: 30. In some embodiments, the MSLN binding protein, according to any one of the above embodiments, is a humanized single domain antibody comprising the sequence of SEQ ID NO: 31. In some embodiments, the MSLN binding protein, according to any one of the above embodiments, is a humanized single domain antibody comprising the sequence of SEQ ID NO: 32. In some embodiments, the MSLN binding protein, according to any one of the above embodiments, is a humanized single domain antibody comprising the sequence of SEQ ID NO: 33. In some embodiments, the MSLN binding protein, according to any one of the above embodiments, is a humanized single domain antibody comprising the sequence of SEQ ID NO: 34. In some embodiments, the MSLN binding protein, according to any one of the above embodiments, is a humanized single domain antibody comprising the sequence of SEQ ID NO: 35. In some embodiments, the MSLN binding protein, according to any one of the above embodiments, is a humanized single domain antibody comprising the sequence of SEQ ID NO: 36. In some embodiments, the MSLN binding protein, according to any one of the above embodiments, is a humanized single domain antibody comprising the sequence of SEQ ID NO: 37. In some embodiments, the MSLN binding protein, according to any one of the above embodiments, is a humanized single domain antibody comprising the sequence of SEQ ID NO: 38. In some embodiments, the MSLN binding protein, according to any one of the above embodiments, is a humanized single domain antibody comprising the sequence of SEQ ID NO: 39. In some embodiments, the MSLN binding protein, according to any one of the above embodiments, is a humanized single domain antibody comprising the sequence of SEQ ID NO: 40. In some embodiments, the MSLN binding protein, according to any one of the above embodiments, is a humanized single domain antibody comprising the sequence of SEQ ID NO: 102. In some embodiments, the MSLN binding protein, according to any one of the above embodiments, is a humanized single domain antibody comprising the sequence of SEQ ID NO: 103. In some embodiments, the MSLN binding protein, according to any one of the above embodiments, is a humanized single domain antibody comprising the sequence of SEQ ID NO: 104. In some embodiments, the MSLN binding protein, according to any one of the above embodiments, is a humanized single domain antibody comprising the sequence of SEQ ID NO: 105.

In some embodiments, the MSLN binding domain is cross-reactive with human and cynomolgus mesothelin. In some embodiments, the MSLN binding domain is specific for human mesothelin. In certain embodiments, the MSLN binding domains disclosed herein bind to human mesothelin with a human Kd (hKd). In certain embodiments, the MSLN binding domains disclosed herein bind to cynomolgus mesothelin with a cyno Kd (cKd). In certain embodiments, the MSLN binding domains disclosed herein bind to both cynomolgus mesothelin and a human mesothelin, with a cyno Kd (cKd) and a human Kd (hKd), respectively. In some embodiments, the MSLN binding protein binds to human and cynomolgus mesothelin with comparable binding affinities (i.e., hKd and cKd values do not differ by more than ±10%). In some embodiments, the hKd and the cKd range from about 0.1 nM to about 500 nM. In some embodiments, the hKd and the cKd range from about 0.1 nM to about 450 nM. In some embodiments, the hKd and the cKd range from about 0.1 nM to about 400 nM. In some embodiments, the hKd and the cKd range from about 0.1 nM to about 350 nM. In some embodiments, the hKd and the cKd range from about 0.1 nM to about 300 nM . In some embodiments, the hKd and the cKd range from about 0.1 nM to about 250 nM. In some embodiments, the hKd and the cKd range from about 0.1 nM to about 200 nM. In some embodiments, the hKd and the cKd range from about 0.1 nM to about 150 nM. In some embodiments, the hKd and the cKd range from about 0.1 nM to about 100 nM. In some embodiments, the hKd and the cKd range from about 0.1 nM to about 90 nM. In some embodiments, the hKd and the cKd range from about 0.2 nM to about 80 nM. In some embodiments, the hKd and the cKd range from about 0.3 nM to about 70 nM. In some embodiments, the hKd and the cKd range from about 0.4 nM to about 50 nM. In some embodiments, the hKd and the cKd range from about 0.5 nM to about 30 nM. In some embodiments, the hKd and the cKd range from about 0.6 nM to about 10 nM. In some embodiments, the hKd and the cKd range from about 0.7 nM to about 8 nM. In some embodiments, the hKd and the cKd range from about 0.8 nM to about 6 nM. In some embodiments, the hKd and the cKd range from about 0.9 nM to about 4 nM. In some embodiments, the hKd and the cKd range from about 1 nM to about 2 nM.

In some embodiments, any of the foregoing MSLN binding domains (e.g., anti-MSLN single domain antibodies of SEQ ID NOs: 1-40) are affinity peptide tagged for ease of purification. In some embodiments, the affinity peptide tag is six consecutive histidine residues, also referred to as 6X-his (SEQ ID NO: 379).

In certain embodiments, the MSLN binding domains of the present disclosure preferentially bind membrane bound mesothelin over soluble mesothelin. Membrane bound mesothelin refers to the presence of mesothelin in or on the cell membrane surface of a cell that expresses mesothelin. Soluble mesothelin refers to mesothelin that is no longer on in or on the cell membrane surface of a cell that expresses or expressed mesothelin. In certain instances, the soluble mesothelin is present in the blood and/or lymphatic circulation in a subject. In one embodiment, the MSLN binding domains bind membrane-bound mesothelin at least 5 fold, 10 fold, 15 fold, 20 fold, 25 fold, 30 fold, 40 fold, 50 fold, 100 fold, 500 fold, or 1000 fold greater than soluble mesothelin. In one embodiment, the MSLN targeting trispecific antigen binding proteins of the present disclosure preferentially bind membrane-bound mesothelin 30 fold greater than soluble mesothelin. Determining the preferential binding of an antigen binding protein to membrane bound MSLN over soluble MSLN can be readily determined using assays well known in the art.

### TriTAC Molecules

Various embodiments of this disclosure provides a trispecific molecule (also referred to herein as a TriTAC molecule) comprising a MSLN binding domain as described herein. In some embodiments, the TriTAC molecule comprises an amino acid sequence as set forth in any one of SEQ ID Nos: 58-86, 98, 100, and 101. In some embodiments, a TriTAC molecule of this disclosure comprises an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOs: 58-86, 98, 100, and 101. In some embodiments, a TriTAC molecule of this disclosure comprises an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the full length of an amino acid sequence selected from SEQ ID NOs: 58-86, 98, 100, and 101. In some embodiments, a TriTAC molecule of this disclosure comprises an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to a fraction of the full length of an amino acid sequence selected from SEQ ID NOs: 58-86, 98, 100, and 101.

Integration into Chimeric Antigen Receptors (CAR)

The MSLN targeting trispecific antigen binding proteins of the present disclosure can, in certain examples, be incorporated into a chimeric antigen receptor (CAR). An engineered immune effector cell, e.g., a T cell or NK cell, can be used to express a CAR that includes an anti-MSLN targeting trispecific protein containing an anti-MSLN single domain antibody as described herein. In one embodiment, the CAR including an anti-MSLN targeting trispecific protein as described herein is connected to a transmembrane domain via a hinge region, and further a costimulatory domain, e.g., a functional signaling domain obtained from OX40, CD27, CD28, CD5, ICAM-1, LFA-1 (CD11a/CD18), ICOS (CD278), or 4-1BB. In some embodiments, the CAR further comprises a sequence encoding a intracellular signaling domain, such as 4-1BB and/or CD3 zeta. Tumor growth reduction properties

In certain embodiments, the MSLN targeting trispecific proteins of the disclosure reduce the growth of tumor cells in vivo when administered to a subject who has tumor cells that express mesothelin. Measurement of the reduction of the growth of tumor cells can be determined by multiple different methodologies well known in the art. Nonlimiting examples include direct measurement of tumor dimension, measurement of excised tumor mass and comparison to control subjects, measurement via imaging techniques (e.g., CT or MRI) that may or may not use isotopes or luminescent molecules (e.g., luciferase) for enhanced analysis, and the like. In specific embodiments, administration of the trispecific proteins of the disclosure results in a reduction of in vivo growth of tumor cells as compared to a control antigen binding agent by at least about 10%, 20%, 30%, 40%, 50%, 60%, 70%, 80%, 90% or 100%, with an about 100% reduction in tumor growth indicating a complete response and disappearance of the tumor. In further embodiments, administration of the trispecific proteins of the disclosure results in a reduction of in vivo growth of tumor cells as compared to a control antigen binding agent by about 50-100%, about 75-100% or about 90-100%. In further embodiments, administration of the trispecific proteins of the disclosure results in a reduction of in vivo growth of tumor cells as compared to a control antigen binding agent by about 50-60%, about 60-70%, about 70-80%, about 80-90%, or about 90-100%.

### MSLN Trispecific Protein Modifications

The MSLN targeting trispecific proteins described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in MSLN targeting trispecific proteins described herein, including the peptide backbone, the amino acid side-chains, and the amino or carboxyl termini. Certain common peptide modifications that are useful for modification of MSLN targeting trispecific proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

### Polynucleotides Encoding MSLN Targeting Trispecific Proteins

Also provided, in some embodiments, are polynucleotide molecules encoding an anti-MSLN trispecific binding protein described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the three binding domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. In the embodiments where the MSLN binding domain is a small molecule, the polynucleotides contain genes encoding the CD3 binding domain and the half-life extension domain. In the embodiments where the half-life extension domain is a small molecule, the polynucleotides contain genes encoding the domains that bind to CD3 and MSLN. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described trispecific antigen-binding protein. Examples of expression vectors for expression in E. coli are pSKK (Le Gall et al., J Immunol Methods. (2004) 285(1):111-27) or pcDNA5 (Invitrogen) for expression in mammalian cells.

Thus, the MSLN targeting trispecific proteins as described herein, in some embodiments, are produced by introducing a vector encoding the protein as described above into a host cell and culturing said host cell under conditions whereby the protein domains are expressed, may be isolated and, optionally, further purified.

### Pharmaceutical Compositions

Also provided, in some embodiments, are pharmaceutical compositions comprising an anti-MSLN trispecific binding protein described herein, a vector comprising the polynucleotide encoding the polypeptide of the MSLN targeting trispecific proteins or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents. A further embodiment provides one or more of the above described MSLN targeting trispecific proteins packaged in lyophilized form, or packaged in an aqueous medium.

In some embodiments of the pharmaceutical compositions, the MSLN targeting trispecific proteins described herein are encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the MSLN trispecific antigen-binding protein is attached to liposomes. In some instances, the MSLN trispecific antigen-binding protein are conjugated to the surface of liposomes. In some instances, the MSLN trispecific antigen-binding protein are encapsulated within the shell of a liposome. In some instances, the liposome is a cationic liposome.

The MSLN targeting trispecific proteins described herein are contemplated for use as a medicament. Administration is effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

In some embodiments, the MSLN targeting trispecific proteins of this disclosure are administered at a dosage of up to 10 mg/kg at a frequency of once a week. In some cases, the dosage ranges from about 1 ng/kg to about 10 mg/kg. In some embodiments, the dose is from about 1 ng/kg to about 10 ng/kg, about 5 ng/kg to about 15 ng/kg, about 12 ng/kg to about 20 ng/kg, about 18 ng/kg to about 30 ng/kg, about 25 ng/kg to about 50 ng/kg, about 35 ng/kg to about 60 ng/kg, about 45 ng/kg to about 70 ng/kg, about 65 ng/kg to about 85 ng/kg, about 80 ng/kg to about 1 μg/kg, about 0.5 μg/kg to about 5 μg/kg, about 2 μg/kg to about 10 μg/kg, about 7 μg/kg to about 15 μg/kg, about 12 μg/kg to about 25 μg/kg, about 20 μg/kg to about 50 μg/kg, about 35 μg/kg to about 70 μg/kg, about 45 μg/kg to about 80 μg/kg, about 65 μg/kg to about 90 μg/kg, about 85 μg/kg to about 0.1 mg/kg, about 0.095 mg/kg to about 10 mg/kg. In some cases, the dosage is about 0.1 mg/kg to about 0.2 mg/kg; about 0.25 mg/kg to about 0.5 mg/kg, about 0.45 mg/kg to about 1 mg/kg, about 0.75 mg/kg to about 3 mg/kg, about 2.5 mg/kg to about 4 mg/kg, about 3.5 mg/kg to about 5 mg/kg, about 4.5 mg/kg to about 6 mg/kg, about 5.5 mg/kg to about 7 mg/kg, about 6.5 mg/kg to about 8 mg/kg, about 7.5 mg/kg to about 9 mg/kg, or about 8.5 mg/kg to about 10 mg/kg. The frequency of administration, in some embodiments, is about less than daily, every other day, less than once a day, twice a week, weekly, once in 7 days, once in two weeks, once in two weeks, once in three weeks, once in four weeks, or once a month. In some cases, the frequency of administration is weekly. In some cases, the frequency of administration is weekly and the dosage is up to 10 mg/kg. In some cases, duration of administration is from about 1 day to about 4 weeks or longer.

### Methods of Treatment

Also provided herein, in some embodiments, are methods and uses for stimulating the immune system of an individual in need thereof comprising administration of an anti-MSLN targeting trispecific protein as described herein. In some instances, the administration of an anti-MSLN targeting trispecific protein described herein induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue.

Also provided herein are methods and uses for a treatment of a disease, disorder or condition associated with a target antigen comprising administering to an individual in need thereof an anti-MSLN targeting trispecific protein described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. Cancers that can be treated, prevented, or managed by the MSLN binding proteins of the present disclosure, and methods of using them, include but are not limited to cancers of an epithelial cell origin. Examples of such cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendothelio sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The MSLN targeting trispecific proteins of the disclosure are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the disclosure. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the skin, lung, colon, breast, prostate, bladder, kidney, pancreas, ovary, or uterus. In other specific embodiments, sarcoma, melanoma, or leukemia is treated or prevented.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the MSLN targeting trispecific proteins as described herein are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (y-rays, X-rays, and/or the directed delivery of radioisotopes, microwaves, UV radiation and the like), gene therapies (e.g., antisense, retroviral therapy and the like) and other immunotherapies. In some embodiments, an anti-MSLN targeting trispecific protein as described herein is administered in combination with anti-diarrheal agents, anti-emetic agents, analgesics, opioids and/or non-steroidal anti-inflammatory agents. In some embodiments, an anti-MSLN targeting trispecific protein as described herein is administered in combination with anti-cancer agents. Nonlimiting examples of anti-cancer agents that can be used in the various embodiments of the disclosure, including pharmaceutical compositions and dosage forms and kits of the disclosure, include: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-nl interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinzolidine sulfate; vinzolidine sulfate; vorozole; zeniplatin; zinostatin; zorubicin hydrochloride. Other examples of anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; HMG-CoA reductase inhibitor (such as but not limited to, Lovastatin, Pravastatin, Fluvastatin, Statin, Simvastatin, and Atorvastatin); loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; Vitaxin®; vorozole; zanoterone; zeniplatin; zilascorb; and zinostatin stimalamer. Additional anti-cancer drugs are 5-fluorouracil and leucovorin. These two agents are particularly useful when used in methods employing thalidomide and a topoisomerase inhibitor. In some embodiments, the anti-MSLN targeting trispecific protein of the present disclosure is used in combination with gemcitabine.

In some embodiments, the anti-MSLN targeting trispecific protein as described herein is administered before, during, or after surgery. Methods of detection of mesothelin expression and diagnosis of mesothelin associated cancer

According to another embodiment of the disclosure, kits for detecting expression of mesothelin in vitro or in vivo are provided. The kits include the foregoing MSLN targeting trispecific proteins (e.g., a trispecific protein containing a labeled anti-MSLN single domain antibody or antigen binding fragments thereof), and one or more compounds for detecting the label. In some embodiments, the label is selected from the group consisting of a fluorescent label, an enzyme label, a radioactive label, a nuclear magnetic resonance active label, a luminescent label, and a chromophore label.

In some cases, mesothelin expression is detected in a biological sample. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. A biological sample is typically obtained from a mammal, such as a human or non-human primate.

In one embodiment, provided is a method of determining if a subject has cancer by contacting a sample from the subject with an anti-MSLN single domain antibody as disclosed herein; and detecting binding of the single domain antibody to the sample. An increase in binding of the antibody to the sample as compared to binding of the antibody to a control sample identifies the subject as having cancer.

In another embodiment, provided is a method of confirming a diagnosis of cancer in a subject by contacting a sample from a subject diagnosed with cancer with an anti-MSLN single domain antibody as disclosed herein; and detecting binding of the antibody to the sample. An increase in binding of the antibody to the sample as compared to binding of the antibody to a control sample confirms the diagnosis of cancer in the subject.

In some examples of the disclosed methods, the MSLN single domain antibody of the trispecific protein is directly labeled.

In some examples, the methods further include contacting a second antibody that specifically binds the anti-MSLN single domain antibody with the sample; and detecting the binding of the second antibody. An increase in binding of the second antibody to the sample as compared to binding of the second antibody to a control sample detects cancer in the subject or confirms the diagnosis of cancer in the subject.

In some cases, the cancer is mesothelioma, prostate cancer, lung cancer, stomach cancer, squamous cell carcinoma, pancreatic cancer, cholangiocarcinoma, triple negative breast cancer or ovarian cancer, or any other type of cancer that expresses mesothelin.

In some examples, the control sample is a sample from a subject without cancer. In particular examples, the sample is a blood or tissue sample.

In some cases, the antibody that binds (for example specifically binds) mesothelin is directly labeled with a detectable label. In another embodiment, the antibody that binds (for example, specifically binds) mesothelin (the first antibody) is unlabeled and a second antibody or other molecule that can bind the antibody that specifically binds mesothelin is labeled. A second antibody is chosen such that it is able to specifically bind the specific species and class of the first antibody. For example, if the first antibody is a llama IgG, then the secondary antibody may be an anti-llama-IgG. Other molecules that can bind to antibodies include, without limitation, Protein A and Protein G, both of which are available commercially. Suitable labels for the antibody or secondary antibody are described above, and include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Nonlimiting examples of suitable prosthetic group complexes include streptavidin/biotin and avidin/biotin. Nonlimiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include 1251, 1311, 35S or 3H.

In an alternative embodiment, mesothelin can be assayed in a biological sample by a competition immunoassay utilizing mesothelin standards labeled with a detectable substance and an unlabeled antibody that specifically binds mesothelin. In this assay, the biological sample, the labeled mesothelin standards and the antibody that specifically bind mesothelin are combined and the amount of labeled mesothelin standard bound to the unlabeled antibody is determined. The amount of mesothelin in the biological sample is inversely proportional to the amount of labeled mesothelin standard bound to the antibody that specifically binds mesothelin.

The immunoassays and method disclosed herein can be used for a number of purposes. In one embodiment, the antibody that specifically binds mesothelin may be used to detect the production of mesothelin in cells in cell culture. In another embodiment, the antibody can be used to detect the amount of mesothelin in a biological sample, such as a tissue sample, or a blood or serum sample. In some examples, the mesothelin is cell-surface mesothelin. In other examples, the mesothelin is soluble mesothelin (e.g., mesothelin in a cell culture supernatant or soluble mesothelin in a body fluid sample, such as a blood or serum sample).

In one embodiment, a kit is provided for detecting mesothelin in a biological sample, such as a blood sample or tissue sample. For example, to confirm a cancer diagnosis in a subject, a biopsy can be performed to obtain a tissue sample for histological examination. Alternatively, a blood sample can be obtained to detect the presence of soluble mesothelin protein or fragment. Kits for detecting a polypeptide will typically comprise a single domain antibody, according to the present disclosure, that specifically binds mesothelin. In some embodiments, an antibody fragment, such as an scFv fragment, a VH domain, or a Fab is included in the kit. In a further embodiment, the antibody is labeled (for example, with a fluorescent, radioactive, or an enzymatic label).

In one embodiment, a kit includes instructional materials disclosing means of use of an antibody that binds mesothelin. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting mesothelin in a biological sample generally includes the steps of contacting the biological sample with an antibody which specifically reacts, under immunologically reactive conditions, to a mesothelin polypeptide. The antibody is allowed to specifically bind under immunologically reactive conditions to form an immune complex, and the presence of the immune complex (bound antibody) is detected directly or indirectly.

Methods of determining the presence or absence of a cell surface marker are well known in the art. For example, the antibodies can be conjugated to other compounds including, but not limited to, enzymes, magnetic beads, colloidal magnetic beads, haptens, fluorochromes, metal compounds, radioactive compounds or drugs. The antibodies can also be utilized in immunoassays such as but not limited to radioimmunoassays (RIAs), ELISA, or immunohistochemical assays. The antibodies can also be used for fluorescence activated cell sorting (FACS). FACS employs a plurality of color channels, low angle and obtuse light-scattering detection channels, and impedance channels, among other more sophisticated levels of detection, to separate or sort cells (see U.S. Pat. No. 5, 061,620). Any of the single domain antibodies that bind mesothelin, as disclosed herein, can be used in these assays. Thus, the antibodies can be used in a conventional immunoassay, including, without limitation, an ELISA, an RIA, FACS, tissue immunohistochemistry, Western blot or imunoprecipitation.

### EXAMPLES

### Example 1

### Methods to Assess Binding and Cytotoxic Activities of Several Exemplary MSLN Targeting Trispecific Antigen Binding Proteins

Protein Production

Sequences of trispecific molecules were cloned into mammalian expression vector pCDNA 3.4 (Invitrogen) preceded by a leader sequence and followed by a 6x Histidine Tag (SEQ ID NO: 379). Expi293F cells (Life Technologies A14527) were maintained in suspension in Optimum Growth Flasks (Thomson) between 0.2 to 8×1e6 cells/ml in Expi 293 media. Purified plasmid DNA was transfected into Expi293 cells in accordance with Expi293 Expression System Kit (Life Technologies, A14635) protocols, and maintained for 4-6 days post transfection. The amount of the exemplary trispecific proteins being tested, in the conditioned media, from the transfected Expi293 cells was quantitated using an Octet instrument with Protein A tips and using a control trispecific protein for a standard curve.

Cytotoxicity Assays

A human T-cell dependent cellular cytotoxicity (TDCC) assay was used to measure the ability of T cell engagers, including trispecific molecules, to direct T cells to kill tumor cells (Nazarian et al. 2015. J Biomol Screen. 20:519-27). In this assay, T cells and target cancer cell line cells are mixed together at a 10:1 ratio in a 384 wells plate, and varying amounts of the trispecific proteins being tested are added. The tumor cell lines are engineered to express luciferase protein. After 48 hours, to quantitate the remaining viable tumor cells, Steady-Glo® Luminescent Assay (Promega) was used.

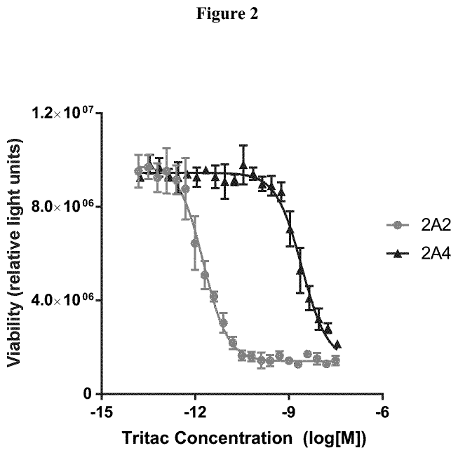

In the instant study, titrations of conditioned media was added to TDCC assays (T cell Dependent Cell Cytotoxicity assays) to assess whether the anti-MSLN single domain antibody was capable of forming a synapse between T cells and a mesothelin expressing ovarian cancer cell line, OVCAR8. Viability of the OVCAR8 cells was measured after 48 hours. It was seen that the trispecific proteins mediated T cell killing. FIG. 2 shows an example cell viability assay with test trispecific proteins 2A2 and 2A4. The EC₅₀ for the TDCC activity of the test trispecific proteins are listed below in Table 1.

TABLE 1
TDCC Activity of MSLN targeting trispecific proteins (TriTAC)

| Anti-MSLN TriTAC | Average EC₅₀ [M] |
|---|---|
| 2A2 | 1.6E−12 |
| 2A4 | 1.9E−09 |
| 11F3 | 2.2E−12 |
| 5D4 | 1.0E−09 |
| 9H2 | 1.1E−12 |
| 5C2 | 1.5E−12 |
| 5G2 | 3.6E−09 |
| 10B3 | 1.4E−12 |
| 2F4 | 7.3E−13 |
| 2C2 | 9.5E−09 |
| 5F2 | 5.3E−12 |
| 7C4 | 1.0E−08 |
| 7F1 | 2.4E−12 |
| 5D2 | 1.4E−11 |
| 6H2 | 2.0E−09 |
| 2D1 | 5.2E−11 |
| 12C2 | 8.0E−13 |
| 3F2 | 2.4E−08 |
| 1H2 | 2.5E−08 |
| 6F3 | 8.2E−10 |
| 2A1 | 1.2E−09 |
| 3G1 | 4.0E−09 |
| 12D1 | 1.1E−09 |
| 5H1 | 5.9E−12 |
| 4A2 | 1.7E−09 |
| 3B4 | 1.8E−12 |
| 7H2 | 5.5E−12 |
| 9F3 | >1E−7 |
| 9B1 | >1E−7 |

Furthermore, it was observed that the TDCC activity of the MSLN targeting trispecific proteins being tested was specific to mesothelin expressing cells, because the trispecific proteins being tested did not mediate T cell killing of LNCaP cells, which do not express mesothelin. The trispecific proteins 2A2, 11F3, 9H2, 5C2, 10B3, 2F4, 5F2, 7F1, 2F4, 5H1, 3B4, and 7H2, in particular did not show ant TDCC activity with the LnCaP cells.

### Example 2

### Xenograft Tumor Model

The MSLN targeting trispecific proteins of the previous example is evaluated in a xenograft model.

Female immune-deficient NOD/scid mice are sub-lethally irradiated (2 Gy) and subcutaneously inoculated with 1×10⁶ NCI-H28cells into their right dorsal flank. When tumors reach 100 to 200 mm³, animals are allocated into 3 treatment groups. Groups 2 and 3 (8 animals each) are intraperitoneally injected with 1.5×10⁷ activated human T-cells. Three days later, animals from Group 3 are subsequently treated with a total of 9 intravenous doses of 50 μg MSLN trispecific antigen-binding protein of Example 1 (qdx9d). Groups 1 and 2 are only treated with vehicle. Body weight and tumor volume are determined for 30 days.

It is expected that animals treated with the MSLN targeting trispecific proteins of the previous examples have a statistically significant delay in tumor growth in comparison to the respective vehicle-treated control group.

### Example 3

### Proof-of-Concept Clinical Trial Protocol for Administration of the MSLN Trispecific Antigen-Binding Protein of Example 1 to Ovarian Cancer Patients

This is a Phase I/II clinical trial for studying the MSLN trispecific antigen-binding protein of Example 1 as a treatment for Ovarian Cancer.

Study Outcomes:

Primary: Maximum tolerated dose of MSLN targeting trispecific proteins of the previous examples

Secondary: To determine whether in vitro response of MSLN targeting trispecific proteins of is the previous examples are associated with clinical response

Phase I

The maximum tolerated dose (MTD) will be determined in the phase I section of the trial.
1.1 The maximum tolerated dose (MTD) will be determined in the phase I section of the trial.
1.2 Patients who fulfill eligibility criteria will be entered into the trial to MSLN targeting trispecific proteins of the previous examples.
1.3 The goal is to identify the highest dose of MSLN targeting trispecific proteins of the previous examples that can be administered safely without severe or unmanageable side effects in participants. The dose given will depend on the number of participants who have been enrolled in the study prior and how well the dose was tolerated. Not all participants will receive the same dose.

Phase II
2.1 A subsequent phase II section will be treated at the MTD with a goal of determining if therapy with therapy of MSLN targeting trispecific proteins of the previous examples results in at least a 20% response rate.
Primary Outcome for the Phase II—To determine if therapy of MSLN targeting trispecific proteins of the previous examples results in at least 20% of patients achieving a clinical response (blast response, minor response, partial response, or complete response)

Eligibility:
Histologically confirmed ovarian cancer according to the current World Health Organisation Classification, 2014
   Surface epithelial—stromal tumors
   Sex cord—stromal tumors
   Germ cell tumors
   Malignant, not otherwise specified
   Age≥18 yrs
   Life expectancy≥6 weeks

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 4

### MH6T TriTAC Directs T Cells to Kill MSLN Expressing Ovarian Cancer Cells

A human T-cell dependent cellular cytotoxicity (TDCC) assay was used to measure the ability of T cell engagers, including trispecific molecules, to direct T cells to kill tumor cells (Nazarian et al. 2015. J Biomol Screen. 20:519-27). The Caov3 cells used in this assay were engineered to express luciferase. T cells from 5 different healthy donors (donor 02, donor 86, donor 41, donor 81, and donor 34) and target cancer cells Caov3 were mixed together and varying amounts of an exemplary trispecific molecule of this disclosure, MH6T TriTAC (SEQ ID NO: 98) was added and the mixture was incubated for 48 hours at 37° C. Caov3 cells and T cells were also incubated for 48 hours at 37° C. with a control trispecific molecule, GFP TriTAC (SEQ ID NO: 99), which targets GFP. After 48 hours, the remaining viable tumor cells were quantified by a luminescence assay.

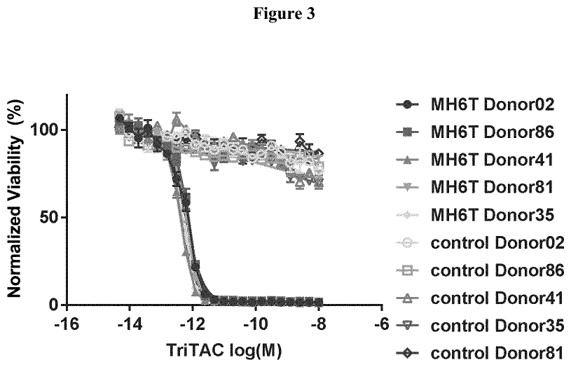

It was observed that the MH6T TriTAC molecule was able to direct the T cells from all 5 healthy donors to kill the target cancer cells Caov3 (as shown in FIG. 3), whereas the control GFP TriTAC molecule was not able to direct the T cells from any of the 5 health donors to kill the Caov3 cells (also shown in FIG. 3).

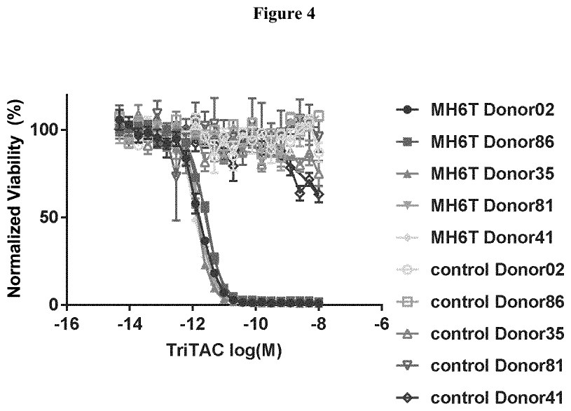

A further assay, using the same protocol as described above, was carried out using OVCAR3 cells. It was observed that the MH6T TriTAC molecule was able to direct the T cells from all 5 healthy donors to kill the target cancer cells OVCAR3 (as shown in FIG. 4), whereas the control GFP TriTAC molecule was not able to direct the T cells from any of the 5 health donors to kill the OVCAR3 cells (also shown in FIG. 4).

The EC₅₀ values for killing of MSLN expressing target cells are listed below in Table II.

TABLE II
EC₅₀ values for MH6T TriTAC directed killing of MSLN-
expressing ovarian cancer cell lines by T cells from
5 different healthy donors. Represented graphs of
the raw data are provided in FIGS. 3 and 4.

| | EC₅₀ values (M) | | | | |
|---|---|---|---|---|---|
| | Donor02 | Donor86 | Donor41 | Donor81 | Donor35 |
| Caov3 | 6.0E−13 | 6.8E−13 | 3.9E−13 | 5.9E−13 | 4.6E−13 |
| Caov4 | 7.3E−12 | 1.1E−11 | 3.7E−12 | 4.7E−12 | 2.2E−12 |
| OVCAR3 | 1.6E−12 | 2.5E−12 | 1.4E−12 | 1.6E−12 | 1.3E−12 |
| OVCAR8 | 2.2E−12 | 3.2E−12 | 1.4E−12 | 1.9E−12 | 1.7E−12 |

### Example 5

### MH6T TriTAC Directs T Cells to Kill Cells Expressing MSLN but not Cells that do not Express MSLN

In this assay, T cells from a healthy donor was incubated with target cancer cells that express MSLN (Caov3 cells, Caov4 cells, OVCAR3 cells, and OVCAR8 cells) or target cancer cells that do not express MSLN (NCI-H510A cells, MDAPCa2b cells). Each of the target cells used in this study were engineered to express luciferase. Varying amounts of the MH6T TriTAC (SEQ ID NO: 98) molecule was added to the mixture of T cells and target cancer cells listed above. The mixture was incubated for 48 hours at 37° C. After 48 hours, the remaining viable target cancer cells were quantified using a luminescent assay.

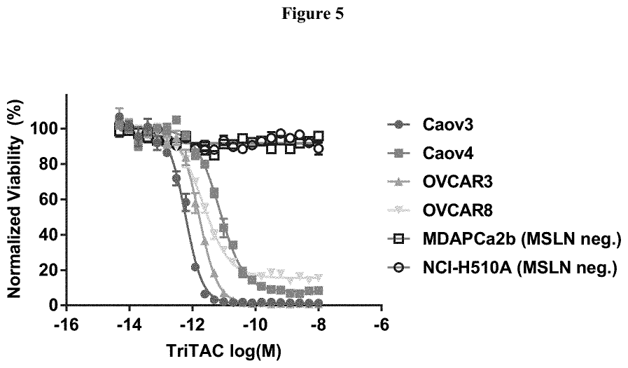

It was observed that the MH6T TriTAC molecule was able to direct T cells to kill MSLN expressing target cancer cells (i.e., Caoc3, Caov4, OVCAR3, and OVCAR8 cells, as shown in FIG. 5). However, the MH6T TriTAC molecule was not able to direct T cells to kill MSLN non-expressing target cancer cells (MDAPCa2b and NCI-H510A cells), also shown in FIG. 5.

The EC₅₀ values for killing of MSLN expressing cancer cells are listed below in Table III

TABLE III
EC₅₀ values for MH6T TriTAC directed T cell
killing of MSLN-expressing cancer cell lines.

| | | EC₅₀ | MSLN sites |
|---|---|---|---|
| Tumor origin | Cell Line | (pM) | per cell |
| Ovarian | Caov3 | 0.6 | 51262 |
| | Caov4 | 7.3 | 101266 |
| | OVCAR3 | 1.6 | 40589 |
| | OVCAR8 | 2.2 | 40216 |
| | SKOV3 | 3.6 | 10617 |
| Pancreatic | Hs766T | 7.8 | 5892 |
| | CaPan2 | 3.2 | 27413 |
| | HPaFII | 15 | 17844 |
| NSCLC | NCI-H596 | 1.5 | 103769 |
| | NCI-H292 | 3.8 | 5977 |
| | NCI-H1563 | 2.6 | 17221 |
| Mesothelioma | NCI-H2052 | 8.0 | not determined |
| | NCI-H2452 | 2.3 | not determined |
| Engineered | HEK293 expressing | 0.9 | 128091 |
| (non-tumor) | human MSLN | | |
| | HEK293 293 expressing | 0.7 | 140683 |
| | cynomolgus MSLN | | |

### Example 6

### MH6T TriTAC Directed T Cells from Cynomolgus Monkeys to Kill Human Ovarian Cancer Cell Lines

In this assay, peripheral blood mononuclear cells (PBMCs; T cells are a component of the PBMCs) from a cynomolgus monkey donor was mixed with target cancer cells that express MSLN (CaOV3 cells and OVCAR3 cells) and varying amounts of the MH6T TriTAC molecule (SEQ ID NO: 98) was added to the mixture, and incubated for 48 hours at 37° C. In parallel, a mixture of cynomolgus PBMCs and MSLN expressing cells, as above, were incubated with varying amounts of a control TriTAC molecule GFP TriTAC (SEQ ID NO: 99) that targets GFP, for 48 hours at 37° C. Target cancer cells used in this assay were engineered to express luciferase. After 48 hours, the remaining viable target cells were quantified using a luminescence assay.

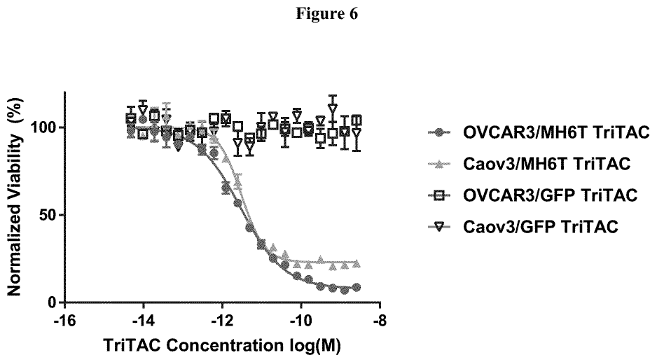

It was observed that the MH6T TriTAC molecule was able to efficiently direct cynomolgus PBMCs to kill MSLN expressing cells (i.e., Caov3 and OVCAR, as shown in FIG. 6, whereas the control GFP TriTAC molecule was not able to direct the cynomolgus PBMCs to kill the cells (also shown in FIG. 6). The EC₅₀ values for the MH6T TriTAC molecule was 2.9 pM for OVCAR3 cells and 3.0 pM for Caov3 cells, which were not significantly different that EC₅₀ values observed with human T cells, as shown in Table II.

### Example 7

### MH6T TriTAC Molecule Directed Killing of MSLN-Expressing NCI-H2052 Mesothelioma Cells by T Cells in the Presence or Absence of Human Serum Albumin

The aim of this study was to assess if binding to human serum albumin (HSA) by MH6T TriTAC molecule impacted the ability of the MH6T TriTAC molecule to direct T cells to kill MSLN-expressing cells. NCI-H₂₀₅₂ mesothelioma cells used in this study were engineered to express luciferase. T cells from a healthy donor and MSLN expressing cells (NCI-H₂₀₅₂) were mixed and varying amounts of the MH6T TriTAC (SEQ ID NO: 98) molecule was added to the mixture. The mixture was incubated for 48 hours at 37° C., in presence or absence of HSA. A mixture of NCI-H₂₀₅₂ cells and T cells were also incubated for 48 hours at 37° C. with a control trispecific molecule, GFP TriTAC (SEQ ID NO: 99), which targets GFP, in presence or absence of HSA. After 48 hours, the remaining viable target cells were quantified using a luminescence assay.

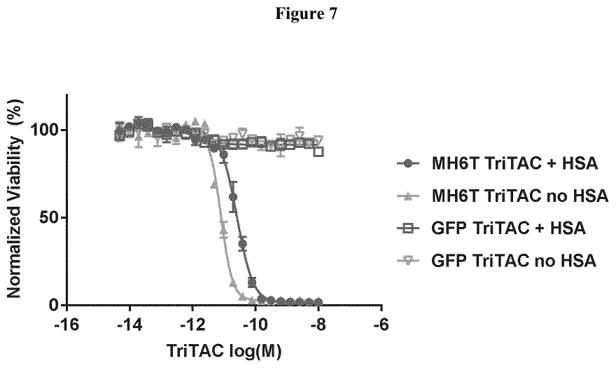

It was observed that the MH6T TriTAC molecule was able to efficiently direct T cells to kill NCI-H₂₀₅₂ cells (as shown in FIG. 7) in presence or absence of HSA, whereas the control GFP TriTAC molecule was not able to do that (also shown in FIG. 7). It was also observed that in presence of HSA, the EC₅₀ value for cell killing was increased by about 3.2 folds (as shown in Table IV).

Further TDCC assays were carried out with the MH6T TriTAC molecule, in presence or absence of 15 mg/ml HSA, with additional MSLN-expressing cells lines and the EC₅₀ values are presented in Table IV.

TABLE IV
EC₅₀ values for MH6T TriTAC directed killing of MSLN-expressing
cancer cells by T cells in the presence or absence of HSA

| | EC₅₀ no | EC₅₀ with | EC₅₀ |
|---|---|---|---|
| Cell line | HSA (pM) | HSA (pM) | shift (fold) |
| OVCAR8 | 2.7 | 8.7 | 3.2 |
| SKOV3 | 3.9 | 11 | 2.8 |
| NCI-H2052 | 8.0 | 26 | 3.2 |
| NCI-H24522 | 2.3 | 6.3 | 2.7 |
| Caov3 | 0.8 | 3.6 | 4.3 |
| OVCAR3 | 1.6 | 3.8 | 2.4 |

### Example 8

### T Cells from 4 Different Donors Secrete TNF-Alpha in the Presence of MH6T TriTAC and MSLN-Expressing Caov4 Cells

The target cancer cells CaOv4 used in this assay were engineered to express luciferase. In this assay, T cells from 4 different healthy donors (donor 02, donor 86, donor 35, and donor 81) and Caov4 cells were mixed together and varying amounts of the MH6T TriTAC molecule (SEQ ID NO: 98) was added and the mixture was incubated for 48 hours at 37° C. Caov4 cells and T cells were also incubated for 48 hours at 37° C. with a control trispecific molecule, GFP TriTAC (SEQ ID NO: 99), which targets GFP. Conditioned medium from the TDCC assay was collected at 48 hours, before measuring the target cancer cell viability, using a luminescence assay. The concentration of TNF-α in the conditioned medium was measured using an AlphaLISA assay kit (Perkin Elmer).

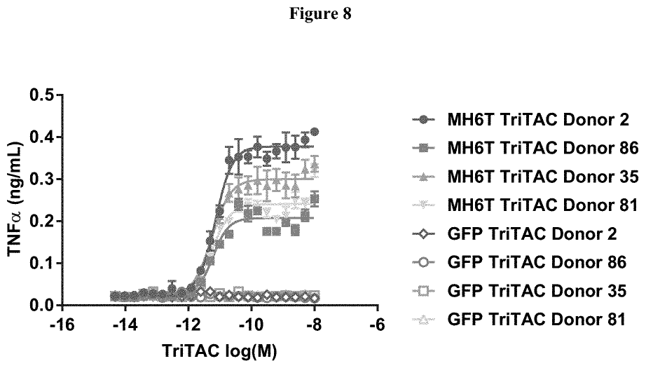

It was observed that TNF-α was secreted into the medium in presence of Caov4 cells and the MH6T TriTAC molecule but not in presence of Caov4 cells and the control GFP TriTAC molecule, as shown in FIG. 8.

Furthermore, efficient killing was observed with T cells from all 4 healthy donors, in presence of the MH6T TriTAC molecule, but not in presence of the control GFP TriTAC molecule. TDCC assays were also set up for additional MSLN expressing cell lines (Caov3 cells, OVCAR3 cells, and OVCAR8 cells) and similar TNF-α expression was observed. The EC₅₀ values for MH6T TriTAC induced expression of TNF-α are presented in Table V. However, when the assay was carried out using cancer cells that do not express MSLN (NCI-H510A cells, or MDAPCa2b cells), no MH6T TriTAC directed secretion of TNF-α was observed (data not shown). Thus, this study demonstrated that the MH6T TriTAC molecule was able to activate T cells in the presence of MSLN-expressing target cancer cells.

TABLE V
EC₅₀ values for MH6T TriTAC molecule induced expression
of TNF-α by T cells from 4 different T cell
donors and 4 different MSLN-expressing cell lines

TNFα EC₅₀ values (M)

| | MH6T | MH6T | MH6T | MH6T |
|---|---|---|---|---|
| | TriTAC | TriTAC | TriTAC | TriTAC |
| | Donor 2 | Donor 86 | Donor 35 | Donor 81 |
| Caov3 | 5.2E−12 | 5.4E−12 | 5.9E−12 | 4.9E−12 |
| Caov4 | 7.2E−12 | 6.0E−12 | 5.5E−12 | 5.5E−12 |
| OVCAR3 | 9.2E−12 | 4.0E−12 | 1.7E−11 | 8.9E−12 |
| OVCAR8 | 1.3E−11 | 9.1E−12 | 5.1E−12 | 5.0E−12 |

### Example 9

### Activation of CD69 Expression on T Cells from 4 Different Donors in Presence of MH6T TriTAC and MSLN-Expressing OVCAR8 Cells

The OVCAR8 cells used in this assay were engineered to express luciferase. In this assay, T cells from 4 different healthy donors (donor 02, donor 86, donor 35, and donor 81) and OVCAR8 cells were mixed together and varying amounts of the MH6T TriTAC molecule (SEQ ID NO: 98) was added and the mixture was incubated for 48 hours at 37° C. OVCAR8 cells and T cells were also incubated for 48 hours at 37° C. with a control trispecific molecule, GFP TriTAC (SEQ ID NO: 99), which targets GFP. After 48 hours, T cells were collected, and CD69 expression on the T cells was measured by flow cytometry.

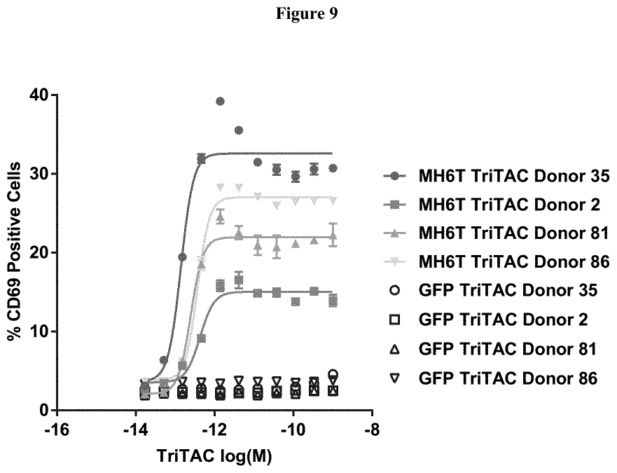

CD69 expression was detected on T cells from all 4 healthy donors in presence of OVCAR8 cells and the MH6T TriTAC molecule but not in presence of the negative control GFP TriTAC and OVCAR8 cells, as shown in FIG. 9. TDCC assays were also set up for additional MSLN expressing cells (Caov3 cells, OVCAR3 cells, and OVCAR8 cells) and similar CD69 expression was observed. The EC₅₀ values for MH6T TriTAC induced activation of CD69 in Caov3 cells and OVCAR8 cells are presented in Table VI.

TABLE VI
EC50 values for activation of CD69 expression on T cells
from 4 different donors in presence of MH6T TriTAC molecule
and MSLN-expressing OVCAR8 cells or Caov3 cells.

| | Caov3 | OVCAR8 |
|---|---|---|
| EC₅₀ table | CD69 (M) | CD69 (M) |
| Donor 35 | ~1.5E−13 | 1.4E−13 |
| Donor 2 | 2.5E−13 | 4.2E−13 |
| Donor 81 | 2.5E−13 | 2.5E−13 |
| Donor 86 | 3.7E−13 | 3.7E−13 |

When the assay was carried out using cancer cells that do not express MSLN (NCI-H510A cells or MDAPCa2b cells), no MH6T induced activation of CD69 was observed (data not shown). Thus, this study demonstrated that the MH6T TriTAC molecule was able to activate T cells in the presence of MSLN-expressing target cancer cells.

### Example 10

### Measurement of MH6T TriTAC Binding to MSLN Expressing/Non-Expressing Cell Lines

For this study, certain target cancer cells that express MSLN (Caov3 cells, CaOV4 cells, OVCAR3 cells, and OVCAR8 cells) and certain cancer cells that do not express MSLN (MDAPCa2b cells, and NCI-H510A cells) were incubated with the MH6T TriTAC molecule (SEQ ID NO: 98) or a control GFP TriTAC molecule (SEQ ID NO: 99). Following incubation, the cells were washed to remove unbound MH6T or GFP TriTAC molecules and further incubated with a secondary antibody conjugated to Alexa Fluor 647, which is able to recognize the anti-albumin domain in the TriTAC molecules,. Binding of the MH6T TriTAC or that of GFP TriTAC to the MSLN expressing or MSLN non-expressing cells was measured by flow cytometry.

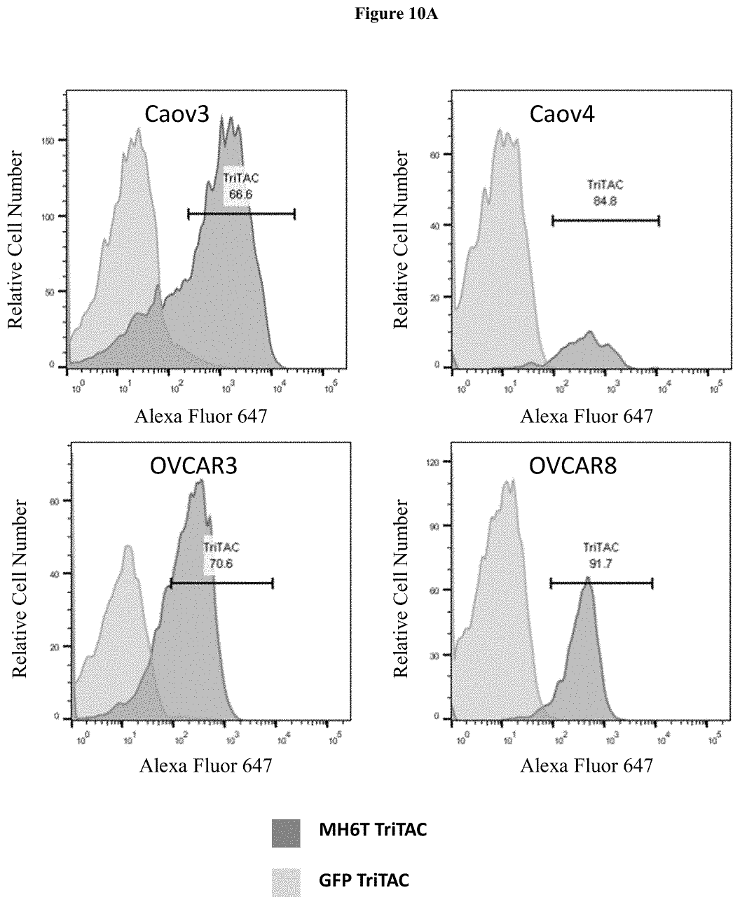

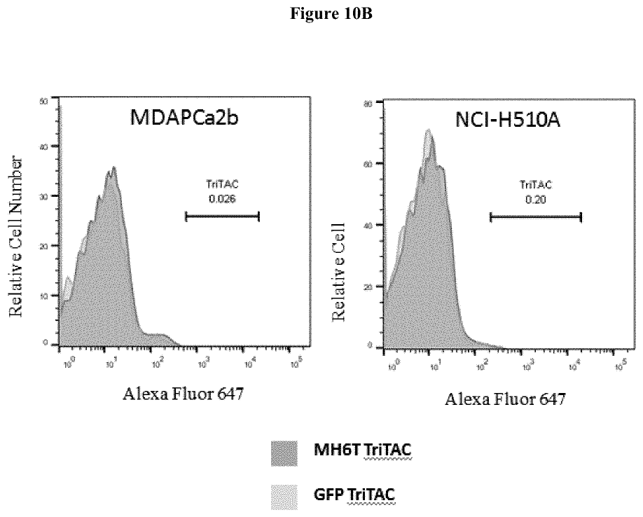

Robust binding of the MH6T TriTAC molecule to cell lines that express MSLN (Caov3, Caov4, OVCAR3, and OVCAR8) was observed, as seen in FIG. 10A (top left panel shows binding of MH6T TriTAC to Caov3 cells; top right panel shows binding of MH6T TriTAC to Caov4 cells; bottom left panel shows binding of MH6T TriTAC to OVCAR3 cells; bottom right panel shows binding of MH6T TriTAC to OVCAR8 cells); and no binding was observed in cell lines that do not express MSLN (left panel shows lack of binding of MH6T TriTAC to MDAPCa2b cells and the right panel shows lack of binding of MH6T TriTAC to NCI-H510A cells), as shown in FIG. 10B. Furthermore, no binding was observed when any of the cell types were incubated with the GFP TriTAC molecule, as shown in both FIGS. 10A and 10B.

### Example 11

### Measurement of MH6T TriTAC Binding to T Cells from Donors

For this study, T cells from 4 health donors were incubated with the MH6T TriTAC molecule (SEQ ID NO: 98) or a buffer, as negative control. Following incubation, the cells were washed to remove unbound MH6T TriTAC molecules and further incubated with an Alexa Fluor 647 conjugated secondary antibody, which is able to recognize the anti-albumin domain in the MH6T TriTAC molecule. Binding of the MH6T TriTAC to the cells was measured by flow cytometry.

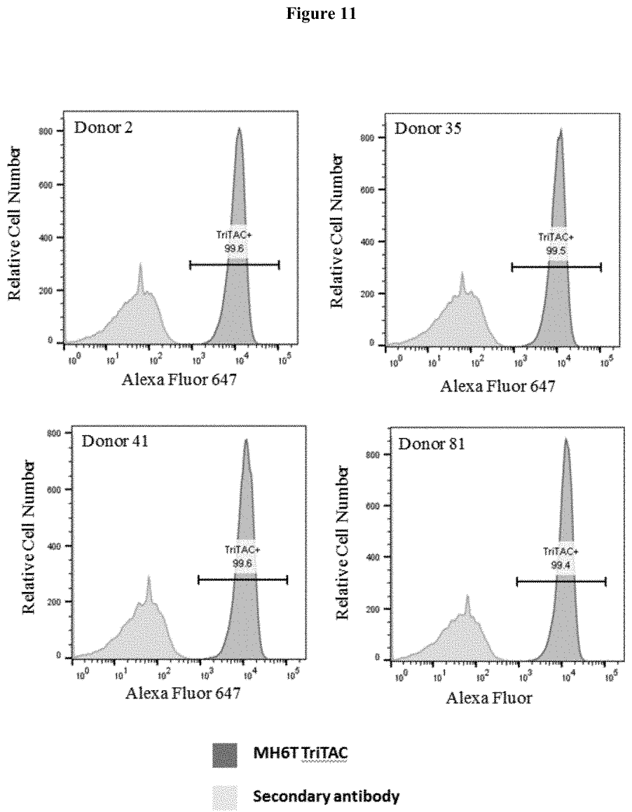

Robust binding of the MH6T TriTAC was observed to T cells from all four donors, treated with the MH6T TriTAC molecule, as shown in FIG. 11 (top left panel shows binding of MH6T TriTAC to T cells from donor 2; top right panel shows binding of MH6T TriTAC to T cells from donor 35; bottom left panel shows binding of MH6T TriTAC to T cells from donor 41; bottom right panel shows binding of MH6T TriTAC to T cells from donor 81).

### Example 12

### Inhibition of Tumor Growth in Mice Treated with MH6T TriTAC Molecule

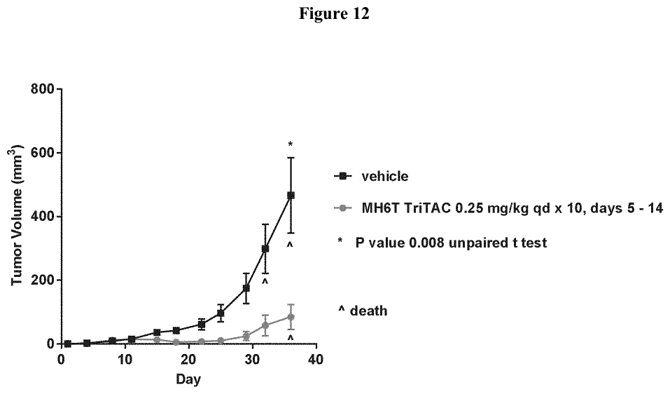

For this study, 10⁷ NCI-H₂₉₂ cells and 10⁷ human PBMCs were co-implanted subcutaneously in two groups of NCG mice (8 mice per group). After 5 days, mice in one group were injected with the MH6T TriTAC molecule (SEQ ID NO: 98), daily for 10 days (days 5-14) at a dose of 0.25 mg/kg; and mice in the other group were injected with a vehicle control. Tumor volumes were measured after every few days and the study was terminated at day 36. Significant inhibition of tumor growth was observed in the mice injected with the MH6T TriTAC molecules, compared to those injected with the vehicle control, as shown in FIG. 12.

### Example 13

### Pharmacokinetics of MH6T TriTAC in Cynomolgus Monkeys

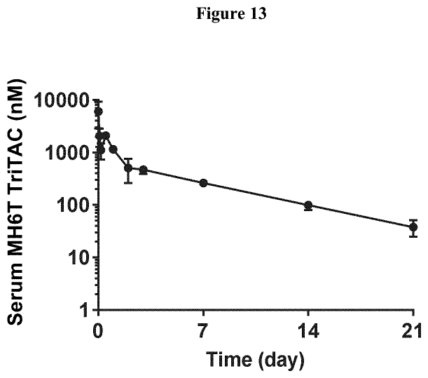

For this study, two cynomolgus monkeys were injected with 10 mg/kg dose of MH6T TriTAC molecule (SEQ ID NO: 98), intravenously, and serum samples were collected at various time points after the injection. The amount of the MH6T TriTAC in the serum was measured using anti-idiotype antibodies recognizing the MH6T TriTAC molecule, in an electrochemiluminescient assay. FIG. 13 shows a plot for the serum M6HT TriTAC levels at various time points. The data was then used to calculate the pharmacokinetic properties of MH6T TriTAC molecule, as provided in Table VII.

TABLE VII
Pharmacokinetic parameters for MH6T TriTAC

| | | Cₘₐₓ | AUC, 0-inf | Clearance | Vss |
|---|---|---|---|---|---|
| Dose Level | Terminal t_{1/2} | (nM) | (hr*nM) | (mL/hr/kg) | (mL/kg) |
| 10 mg/kg | 112 | 6,130 | 355,000 | 0.58 | 70.0 |

### Example 14

### Optimization of CDR Binding Affinity for Maximum Activity and Exposure of Two Exemplary Trispecific Molecules of this Disclosure

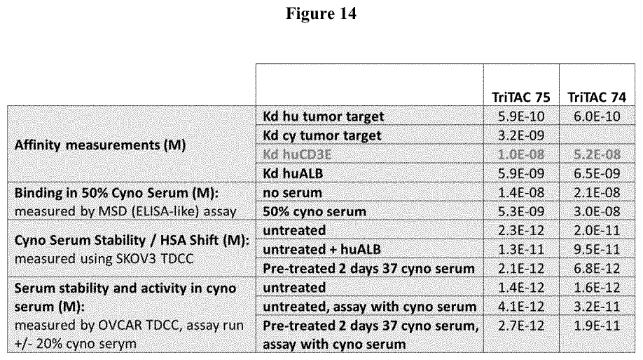

CD3ε, MSLN, and albumin binding affinities of two exemplary trispecific molecules of this disclosure, TriTAC 74 (SEQ ID NO: 100) and TriTAC 75 (SEQ ID NO: 101) were measured for this study. It was observed that TriTAC74 was about 5 times more potent in binding human CD3c than TriTAC75, even though the binding affinities of the two molecules were similar for the tumor target (MSLN) and albumin, as shown in FIG. 14. Additionally, TDCC assays were carried out with the TriTAC 74 and TriTAC 75 molecules, using SKOV3 and OVCAR cells. FIG. 14 shows the EC₅₀ values obtained in the TDCC assays.

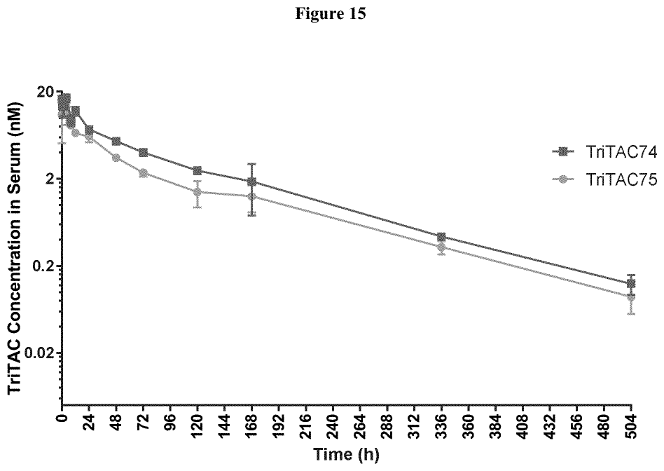

The difference in CD3ε affinity was found to lead to approximately 30% to 50% increase in AUC, in TriTAC 74, compared to TriTAC 75, as measured in a pharmacokinetic assay after injecting cynomolgus monkeys with the TriTAC molecules (at an intravenous bolus dose of 0.02 mg/kg), provided in Table VIII Serum levels of the TriTAC molecules were measured at various time points after the injection, using a Meso Scale Discovery (MSD) assay with anti-idiotype antibodies. The MSD assay was carried out using n=2 replicates. Serum concentrations observed in MSD assay are shown in FIG. 15 and the pharmacokinetic parameters are listed in Table VIII

TABLE VIII
Pharmacokinetic parameters for TriTAC 74 and TriTAC 75

| | Terminal | AUC, 0-last | AUC, 0-inf | Clearance | Vss |
|---|---|---|---|---|---|
| TriTAC | t_{1/2} | (hr*nM) | (hr*nM) | (mL/hr/kg) | (mL/kg) |
| 74 | 84.9 | 1030 | 1050 | 0.367 | 36.8 |
| 75 | 89.4 | 715 | 727 | 0.522 | 54.2 |

SEQUENCE TABLE

| | Exemplary | |
|---|---|---|
| | MSLN | |
| | binding | |
| Sequence | trispecific | |
| ID No. | protein | Sequence |
| SEQ ID | 9B1 | QVQLVESGGGLVQPGGSLRLSCAASGRTFSVRGMAWYRQAGNNRALVATMNPDGFPN |
| NO: 1 | | YADAVKGRFTISWDIAENTVYLQMNSLNSEDTTVYYCNSGPYWGQGTQVTVSS |
| SEQ ID | 9F3 | QVQLVESGGGLVQAGGSLRLSCAASGSIPSIEQMGWYRQAPGKQRELVAALTSGGRA |
| NO: 2 | | NYADSVKGRFTISGDNVRNMVYLQMNSLKPEDTAIYYCSAGRFKGDYAQRSGMDYWG |
| | | KGTLVTVSS |
| SEQ ID | 7H2 | QVQLVESGGGLVQAGGSLRLSCAFSGTTYTEDLMSWYRQAPGKQRTVVASISSDGRT |
| NO: 3 | | SYADSVRGRFTISGENGKNTVYLQMNSLKLEDTAVYYCLGQRSGVRAFWGQGTQVTV |
| | | SS |
| SEQ ID | 3B4 | QVQLVESGGGLVQAGGSLRLSCVASGSTSNINNMRWYRQAPGKERELVAVITRGGYA |
| NO: 4 | | IYLDAVKGRFTISRDNANNAIYLEMNSLKPEDTAVYVCNADRVEGTSGGPQLRDYFG |
| | | QGTQVTVSS |
| SEQ ID | 4A2 | QVQLVESGGGLVQAGGSLRLSCAASGSTFGINAMGWYRQAPGKQRELVAVISRGGST |
| NO: 5 | | NYADSVKGRFTISRDNAENTVSLQMNTLKPEDTAVYFCNARTYTRHDYWGQGTQVTV |
| | | SS |
| SEQ ID | 12D1 | QVRLVESGGGLVQAGGSLRLSCAASISAFRLMSVRWYRQDPSKQREWVATIDQLGRT |
| NO: 6 | | NYADSVKGRFAISKDSTRNTVYLQMNMLRPEDTAVYYCNAGGGPLGSRWLRGRHWGQ |
| | | GTQVTVSS |
| SEQ ID | 3G1 | QVRLVESGGGLVQAGESLRLSCAASGRPFSINTMGWYRQAPGKQRELVASISSSGDF |
| NO: 7 | | TYTDSVKGRFTISRDNAKNTVYLQMNSLKPEDTAVYYCNARRTYLPRRFGSWGQGTQ |
| | | VTVSS |
| SEQ ID | 2A1 | QVQPVESGGGLVQPGGSLRLSCVVSGSDFTEDAMAWYRQASGKERESVAFVSKDGKR |
| NO: 8 | | ILYLDSVRGRFTISRDIDKKTVYLQMDNLKPEDTGVYYCNSAPGAARNYWGQGTQVT |
| | | VSS |
| SEQ ID | 6F3 | QVQPVESGGGLVQPGGSLRLSCVVSGSDFTEDAMAWYRQASGKERESVAFVSKDGKR |
| NO: 9 | | ILYLDSVRGRFTISRDIYKKTVYLQMDNLKPEDTGVYYCNSAPGAARNVWGQGTQVT |
| | | VSS |
| SEQ ID | 1H2 | EVQLVESGGGLVQPGNSLRLSCAASGFTFSSFGMSWVRQAPGKGLEWVSSISGSGSD |
| NO: 10 | | TLYADSVKGRFTISRDNAKTTLYLQMNSLRPEDTAVYYCTIGGSLSRSSQGTLVTVS |
| | | S |
| SEQ ID | 3F2 | QVQIVESGGGLVQAGGSLRLSCVASGLTYSIVAVGWYRQAPGKEREMVADISPVGNT |
| NO: 11 | | NYADSVKGRFTISKENAKNTVYLQMNSLKPEDTAVYYCHIVRGWLDERPGPGPIVYW |
| | | GQGTQVTVSS |
| SEQ ID | 12C2 | QVQLVESGGGLVQTGGSLRLSCAASGLTFGVYGMEWFRQAPGKQREWVASHTSTGYV |
| NO: 12 | | YYRDSVKGRFTISRDNAKSTVYLQMNSLKPEDTAIYYCKANRGSYEYWGQGTQVTVS |
| | | S |
| SEQ ID | 2D1 | QVQLVESGGGLVQAGGSLRLSCAASTTSSINSMSWYRQAQGKQREPVAVITDRGSTS |
| NO: 13 | | YADSVKGRFTISRDNAKNTVYLQMNSLKPEDTAIYTCHVIADWRGYWGQGTQVTVSS |
| SEQ ID | 6H2 | QVQLVESGGGLVQAGGSLRLSCAASGRTLSRYAMGWFRQAPGKERQFVAAISRSGGT |
| NO: 14 | | TRYSDSVKGRFTISRDNAANTFYLQMNNLRPDDTAVYYCNVRRRGWGRTLEYWGQGT |
| | | QVTVSS |
| SEQ ID | 5D2 | QVQLGESGGGLVQAGGSLRLSCAASGSIFSPNAMIWHRQAPGKQREPVASINSSGST |
| NO: 15 | | NYGDSVKGRFTVSRDIVKNTMYLQMNSLKPEDTAVYYCSYSDFRRGTQYWGQGTQVT |
| | | VSS |
| SEQ ID | 7C4 | QVQLVESGGGLVPSGGSLRLSCAASGATSAITNLGWYRRAPGQVREMVARISVREDK |
| NO: 16 | | EDYEDSVKGRFTISRDNTQNLVYLQMNNLQPHDTAIYYCGAQRWGRGPGTTWGQGTQ |
| | | VTVSS |
| SEQ ID | 5F2 | QVQLVESGGGLVQAGGSLRLSCAASGSTFRIRVMRWYRQAPGTERDLVAVISGSSTY |
| NO: 17 | | YADSVKGRFTISRDNAKNTLYLQMNNLKPEDTAVYYCNADDSGIARDYWGQGTQVTV |
| | | SS |
| SEQ ID | 2C2 | QVQLVESGGGLVQAGESRRLSCAVSGDTSKFKAVGWYRQAPGAQRELLAWINNSGVG |
| NO: 18 | | NTAESVKGRFTISRDNAKNTVYLQMNRLTPEDTDVYYCRFYRREGINKNYWGQGTQV |
| | | TVSS |
| SEQ ID | 5G2 | QVQLVESGGGLVQAGGSLRLSCAASGSTFGNKPMGWYRQAPGKQRELVAVISSDGGS |
| NO: 19 | | TRYAALVKGRFTISRDNAKNTVYLQMESLVAEDTAVYYCNALRTYYLNDPVVESWGQ |
| | | GTQVTVSS |
| SEQ ID | 9H2 | QVQLVESGGGLVQAGGSLRLSCAASGSTSSINTMYWYRQAPGKERELVAFISSGGST |
| NO: 20 | | NVRDSVKGRFSVSRDSAKNIVYLQMNSLTPEDTAVYYCNTYIPLRGTLHDYWGQGTQ |
| | | VTVSS |
| SEQ ID | 5D4 | QVQLVESGGGLVQAGGSLRLSCVASGRTDRITTMGWYRQAPGKQRELVATISNRGTS |
| NO: 21 | | NYANSVKGRFTISRDNAKNTVYLQMNSLKPEDTAVYYCNARKWGRNYWGQGTQVTVS |
| | | S |
| SEQ ID | 2A4 | QVQLVESGGGLVQARGSLRLSCTASGRTIGINDMAWYRQAPGNQRELVATITKGGTT |
| NO: 22 | | DYADSVDGRFTISRDNAKNTVYLQMNSLKPEDTAVYYCNTKRREWAKDFEYWGQGTQ |
| | | VTVSS |
| SEQ ID | 7F1 | QVQLVESGGGLVQAGGSLRLSCAASAIGSINSMSWYRQAPGKQREPVAVITDRGSTS |
| NO: 23 | | YADSVKGRFTISRDNAKNTVYLQMNSLKPEDTAIYTCHVIADWRGYWGQGTQVTVSS |
| SEQ ID | 5C2 | QVQLVESGGGLVQAGGSLRLSCAASGSTSSINTMYWFRQAPGEERELVATINRGGST |
| NO: 24 | | NVRDSVKGRFSVSRDSAKNIVYLQMNRLKPEDTAVYYCNTYIPYGGTLHDFWGQGTQ |
| | | VTVSS |
| SEQ ID | 2F4 | QVQLVESGGGLVQAGGSLRLSCTTSTTFSINSMSWYRQAPGNQREPVAVITNRGTTS |
| NO: 25 | | YADSVKGRFTISRDNARNTVYLQMDSLKPEDTAIYTCHVIADWRGYWGQGTQVTVSS |
| SEQ ID | 2A2 | QVQLVESGGGLVQAGGSLTLSCAASGSTFSIRAMRWYRQAPGTERDLVAVIYGSSTY |
| NO: 26 | | YADAVKGRFTISRDNAKNTLYLQMNNLKPEDTAVYYCNADTIGTARDYWGQGTQVTV |
| | | SS |
| SEQ ID | 11F3 | QVQLVESGGGLVQAGGSLRLSCVASGRTSTIDTMYWHRQAPGNERELVAYVTSRGTS |
| NO: 27 | | NVADSVKGRFTISRDNAKNTAYLQMNSLKPEDTAVYYCSVRTTSYPVDFWGQGTQVT |
| | | VSS |
| SEQ ID | 10B3 | QVQLVESGGGLVQAGGSLRLSCAASGSTSSINTMYWYRQAPGKERELVAFISSGGST |
| NO: 28 | | NVRDSVKGRFSVSRDSAKNIVYLQMNSLKPEDTAVYYCNTYIPYGGTLHDFWGQGTQ |
| | | VTVSS |
| SEQ ID | 5H1 | QVQLVESGGGLVQPGGSLRLSCAASGGDWSANFMYWYRQAPGKQRELVARISGRGVV |
| NO: 29 | | DYVESVKGRFTISRDNAKNTVYLQMNSLKPEDTAVYYCAVASYWGQGTQVTVSS |
| SEQ ID | MH1 | EVQLVESGGGLVQPGGSLRLSCAASGGDWSANFMYWYRQAPGKQRELVARISGRGVV |
| NO: 30 | (exemplary | DYVESVKGRFTISRDNSKNTLYLQMNSLRAEDTAVYYCAVASYWGQGTLVTVSS |
| | humanized | |
| | version of | |
| | 5H1) | |
| SEQ ID | MH2 | EVQLVESGGGLVQPGGSLRLSCAASGGDWSANFMYWVRQAPGKGLEWVSRISGRGVV |
| NO: 31 | (exemplary | DYVESVKGRFTISRDNSKNTLYLQMNSLRAEDTAVYYCAVASYWGQGTLVTVSS |
| | humanized | |
| | version of | |
| | 5H1) | |
| SEQ ID | MH3 | EVQLVESGGGLVQAGGSLRLSCAASGSTSSINTMYWYRQAPGKERELVAFISSGGST |
| NO: 32 | (exemplary | NVRDSVKGRFTISRDNSKNTLYLQMNSLRAEDTAVYYCNTYIPYGGTLHDFWGQGTL |
| | humanized | VTVSS |
| | version of | |
| | 10B3) | |
| SEQ ID | MH4 | EVQLVESGGGLVQPGGSLRLSCAASGSTSSINTMYWYRQAPGKERELVAFISSGGST |
| NO: 33 | (exemplary | NVRDSVKGRFTISRDNSKNTLYLQMNSLRAEDTAVYYCNTYIPYGGTLHDFWGQGTL |
| | humanized | VTVSS |
| | version of | |
| | 10B3) | |
| SEQ ID | MH5 | EVQLVESGGGLVQPGGSLRLSCAASGSTSSINTMYWVRQAPGKGLEWVSFISSGGST |
| NO: 34 | (exemplary | NVRDSVKGRFTISRDNSKNTLYLQMNSLRAEDTAVYYCNTYIPYGGTLHDFWGQGTL |
| | humanized | VTVSS |
| | version of | |
| | 10B3) | |
| SEQ ID | MH6-GG | QVQLVESGGGVVQAGGSLRLSCAASGSTESIRAMRWYRQAPGTERDLVAVIYGSSTY |
| NO: 35 | (exemplary | YADAVKGRFTISRDNSKNTLYLQMNSLRAEDTAVYYCNADTIGTARDYWGQGTLVTV |
| | humanized | SSGG |
| | version of | |
| | 2A2) | |
| SEQ ID | MH7-GG | QVQLVESGGGVVQPGGSLRLSCAASGSTESIRAMRWYRQAPGKERELVAVIYGSSTY |
| NO: 36 | (exemplary | YADAVKGRFTISRDNSKNTLYLQMNSLRAEDTAVYYCNADTIGTARDYWGQGTLVTV |
| | humanized | SSGG |
| | version of | |
| | 2A2) | |
| SEQ ID | MH8-GG | QVQLVESGGGVVQPGGSLRLSCAASGSTESIRAMRWVRQAPGKGLEWVSVIYGSSTY |
| NO: 37 | (exemplary | YADAVKGRFTISRDNSKNTLYLQMNSLRAEDTAVYYCNADTIGTARDYWGQGTLVTV |
| | humanized | SSGG |
| | version of | |
| | 2A2) | |
| SEQ ID | MH9 | EVQLVESGGGLVQAGGSLRLSCVASGRTSTIDTMYWHRQAPGNERELVAYVTSRGTS |
| NO: 38 | (exemplary | NVADSVKGRFTISRDNSKNTLYLQMNSLRAEDTAVYYCSVRTTSYPVDFWGQGTLVT |
| | humanized | VS |
| | version of | |
| | 11F3) | |
| SEQ ID | MH10 | EVQLVESGGGLVQPGGSLRLSCAASGRTSTIDTMYWHRQAPGKERELVAYVTSRGTS |
| NO: 39 | (exemplary | NVADSVKGRFTISRDNSKNTLYLQMNSLRAEDTAVYYCSVRTTSYPVDFWGQGTLVT |
| | humanized | VSS |
| | version of | |
| | 11F3) | |
| SEQ ID | MH11 | EVQLVESGGGLVQPGGSLRLSCAASGRTSTIDTMYWVRQAPGKGLEWVSYVTSRGTS |
| NO: 40 | (exemplary | NVADSVKGRFTISRDNSKNTLYLQMNSLRAEDTAVYYCSVRTTSYPVDFWGQGTLVT |
| | humanized | VSS |
| | version of | |
| | 11F3) | |
| SEQ ID | Exemplary | ESGGGLV |
| NO: 41 | conserved | |
| | region in | |
| | MSLN | |
| | binding | |
| | domain | |
| SEQ ID | Exemplary | LSC |
| NO: 42 | conserved | |
| | region in | |
| | MSLN | |
| | binding | |
| | domain | |
| SEQ ID | Exemplary | GRF |
| NO: 43 | conserved | |
| | region in | |
| | MSLN | |
| | binding | |
| | domain | |
| SEQ ID | Exemplary | VTVSS |
| NO: 44 | conserved | |
| | region in | |
| | MSLN | |
| | binding | |
| | domain | |
| SEQ ID | Exemplary | QLVESGGG |
| NO: 45 | conserved | |
| | region in | |
| | MSLN | |
| | binding | |
| | domain | |
| SEQ ID | Exemplary | GGSLRLSCAASG |
| NO: 46 | conserved | |
| | region in | |
| | MSLN | |
| | binding | |
| | domain | |
| SEQ ID | Exemplary | ASG |
| NO: 47 | conserved | |
| | region in | |
| | MSLN | |
| | binding | |
| | domain | |
| SEQ ID | Exemplary | RQAPG |
| NO: 48 | conserved | |
| | region in | |
| | MSLN | |
| | binding | |
| | domain | |
| SEQ ID | Exemplary | VKGRFTISRDNSKNTLYLQMNSLRAEDTAVYYC |
| NO: 49 | conserved | |
| | region in | |
| | MSLN | |
| | binding | |
| | domain | |
| SEQ ID | Exemplary | WGQGTLVTVSS |
| NO: 50 | conserved | |
| | region in | |
| | MSLN | |
| | binding | |
| | domain | |
| SEQ ID | Exemplary | GRTFSVRGMA |
| NO: 51 | CDR1 of | |
| | MSLN | |
| | binding | |
| | domain | |
| SEQ ID | Exemplary | INSSGSTNYG |
| NO: 52 | CDR2 of | |
| | MSLN | |
| | binding | |
| | domain | |
| SEQ ID | Exemplary | NAGGGPLGSR |
| NO: 53 | CDR3 of | |
| | MSLN | |
| | binding | |
| | domain | |
| SEQ ID | Exemplary | GGDWSANFMY |
| NO: 54 | CDR1 of | |
| | MSLN | |
| | binding | |
| | domain | |
| SEQ ID | Exemplary | ISSGGSTNVR |
| NO: 55 | CDR2 of | |
| | MSLN | |
| | binding | |
| | domain | |
| SEQ ID | Exemplary | NADTIGTARD |
| NO: 56 | CDR3 of | |
| | MSLN | |
| | binding | |
| | domain | |
| SEQ ID | Mesothelin | MALPTARPLLGSCGTPALGSLLFLLFSLGWVQPSRTLAGETGQEAAPLDGVLANPPN |
| NO: 57 | protein | ISSLSPRQLLGFPCAEVSGLSTERVRELAVALAQKNVKLSTEQLRCLAHRLSEPPED |
| | sequence | LDALPLDLLLFLNPDAFSGPQACTRFFSRITKANVDLLPRGAPERQRLLPAALACWG |
| | | VRGSLLSEADVRALGGLACDLPGRFVAESAEVLLPRLVSCPGPLDQDQQEAARAALQ |
| | | GGGPPYGPPSTWSVSTMDALRGLLPVLGQPIIRSIPQGIVAAWRQRSSRDPSWRQPE |
| | | RTILRPRERREVEKTACPSGKKAREIDESLIFYKKWELEACVDAALLATQMDRVNAI |
| | | PFTYEQLDVLKHKLDELYPQGYPESVIQHLGYLFLKMSPEDIRKWNVTSLETLKALL |
| | | EVNKGHEMSPQAPRRPLPQVATLIDRFVKGRGQLDKDTLDTLTAFYPGYLCSLSPEE |
| | | LSSVPPSSIWAVRPQDLDTCDPRQLDVLYPKARLAFQNMNGSEYFVKIQSFLGGAPT |
| | | EDLKALSQQNVSMDLATFMKLRTDAVLPLTVAEVQKLLGPHVEGLKAEERHRPVRDW |
| | | ILRQRQDDLDTLGLGLQGGIPNGYLVLDLSMQEALSGTPCLLGPGPVLTVLALLLAS |
| | | TLA |
| SEQ ID | 9B1 TriTAC | QVQLVESGGGLVQPGGSLRLSCAASGRTFSVRGMAWYRQAGNNRALVATMNPDGFPN |
| NO: 58 | | YADAVKGRFTISWDIAENTVYLQMNSLNSEDTTVYYCNSGPYWGQGTQVTVSSGGGG |
| | | SGGGSEVQLVESGGGLVQPGNSLRLSCAASGFTFSSFGMSWVRQAPGKGLEWVSSIS |
| | | GSGSDTLYADSVKGRFTISRDNAKTTLYLQMNSLRPEDTAVYYCTIGGSLSRSSQGT |
| | | LVTVSSGGGGSGGGSEVQLVESGGGLVQPGGSLKLSCAASGFTENKYAMNWVRQAPG |
| | | KGLEWVARIRSKYNNYATYYADSVKDRFTISRDDSKNTAYLQMNNLKTEDTAVYYCV |
| | | RHGNEGNSYISYWAYWGQGTLVTVSSGGGGSGGGGSGGGGSQTVVTQEPSLTVSPGG |
| | | TVTLTCGSSTGAVTSGNYPNWVQQKPGQAPRGLIGGTKFLAPGTPARFSGSLLGGKA |
| | | ALTLSGVQPEDEAEYYCVLWYSNRWVFGGGTKLTVLHHHHHH |
| SEQ ID | 9F3 TriTAC | QVQLVESGGGLVQAGGSLRLSCAASGSIPSIEQMGWYRQAPGKQRELVAALTSGGRA |
| NO: 59 | | NYADSVKGRFTISGDNVRNMVYLQMNSLKPEDTAIYYCSAGRFKGDYAQRSGMDYWG |
| | | KGTLVTVSSGGGGSGGGSEVQLVESGGGLVQPGNSLRLSCAASGFTFSSFGMSWVRQ |
| | | APGKGLEWVSSISGSGSDTLYADSVKGRFTISRDNAKTTLYLQMNSLRPEDTAVYYC |
| | | TIGGSLSRSSQGTLVTVSSGGGGSGGGSEVQLVESGGGLVQPGGSLKLSCAASGFTF |
| | | NKYAMNWVRQAPGKGLEWVARIRSKYNNYATYYADSVKDRFTISRDDSKNTAYLQMN |
| | | NLKTEDTAVYYCVRHGNFGNSYISYWAYWGQGTLVTVSSGGGGSGGGGSGGGGSQTV |
| | | VTQEPSLTVSPGGTVTLTCGSSTGAVTSGNYPNWVQQKPGQAPRGLIGGTKFLAPGT |
| | | RARFSGSLLGGKAALTLSGVQPEDEAEYYCVLWYSNRWVEGGGTKLTVLHHHHHH |
| SEQ ID | 7H2 TriTAC | QVQLVESGGGLVQAGGSLRLSCAFSGTTYTEDLMSWYRQAPGKQRTVVASISSDGRT |
| NO: 60 | | SYADSVRGRFTISGENGKNTVYLQMNSLKLEDTAVYYCLGQRSGVRAFWGQGTQVTV |
| | | SSGGGGSGGGSEVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVYYCTIGGSLS |
| | | RSSQGTLVTVSSGGGGSGGGSEVQLVESGGGLVQPGGSLKLSCAASGFTENKYAMNW |
| | | VRQAPGKGLEWVARIRSKYNNYATYYADSVKDRFTISRDDSKNTAYLQMNNLKTEDT |
| | | AVYYCVRHGNEGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGS |
| | | LLGGKAALTLSGVQPEDEAEYYCVLWYSNRWVFGGGTKLTVLHHHHHH |
| SEQ ID | 3B4 TriTAC | QVQLVESGGGLVQAGGSLRLSCVASGSTSNINNMRWYRQAPGKERELVAVITRGGYA |
| NO: 61 | | IYLDAVKGRFTISRDNANNAIYLEMNSLKPEDTAVYVCNADRVEGTSGGPQLRDYFG |
| | | QGTQVTVSSGGGGSGGGSEVQLVESGGGLVQPGNSLRLSCAASGFTFSSFGMSWVRQ |
| | | APGKGLEWVSSISGSGSDTLYADSVKGRFTISRDNAKTTLYLQMNSLRPEDTAVYYC |
| | | TIGGSLSRSSQGTLVTVSSGGGGSGGGSEVQLVESGGGLVQPGGSLKLSCAASGFTF |
| | | NKYAMNWVRQAPGKGLEWVARIRSKYNNYATYYADSVKDRFTISRDDSKNTAYLQMN |
| | | NLKTEDTAVYYCVRHGNFGNSYISYWAYWGQGTLVTVSSGGGGSGGGGSGGGGSQTV |
| | | VTQEPSLTVSPGGTVTLTCGSSTGAVTSGNYPNWVQQKPGQAPRGLIGGTKFLAPGT |
| | | RARFSGSLLGGKAALTLSGVQPEDEAEYYCVLWYSNRWVEGGGTKLTVLHHHHHH |
| SEQ ID | 4A2 TriTAC | QVQLVESGGGLVQAGGSLRLSCAASGSTFGINAMGWYRQAPGKQRELVAVISRGGST |
| NO: 62 | | NYADSVKGRFTISRDNAENTVSLQMNTLKPEDTAVYFCNARTYTRHDYWGQGTQVTV |
| | | SSGGGGSGGGSEVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVYYCTIGGSLS |
| | | RSSQGTLVTVSSGGGGSGGGSEVQLVESGGGLVQPGGSLKLSCAASGFTENKYAMNW |
| | | VRQAPGKGLEWVARIRSKYNNYATYYADSVKDRFTISRDDSKNTAYLQMNNLKTEDT |
| | | AVYYCVRHGNEGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGS |
| | | LLGGKAALTLSGVQPEDEAEYYCVLWYSNRWVFGGGTKLTVLHHHHHH |
| SEQ ID | 12D1 TriTAC | QVRLVESGGGLVQAGGSLRLSCAASISAFRLMSVRWYRQDPSKQREWVATIDQLGRT |
| NO: 63 | | NYADSVKGRFAISKDSTRNTVYLQMNMLRPEDTAVYYCNAGGGPLGSRWLRGRHWGQ |
| | | GTQVTVSSGGGGSGGGSEVQLVESGGGLVQPGNSLRLSCAASGFTFSSFGMSWVRQA |
| | | PGKGLEWVSSISGSGSDTLYADSVKGRFTISRDNAKTTLYLQMNSLRPEDTAVYYCT |
| | | IGGSLSRSSQGTLVTVSSGGGGSGGGSEVQLVESGGGLVQPGGSLKLSCAASGFTFN |
| | | KYAMNWVRQAPGKGLEWVARIRSKYNNYATYYADSVKDRFTISRDDSKNTAYLQMNN |
| | | LKTEDTAVYYCVRHGNEGNSYISYWAYWGQGTLVTVSSGGGGSGGGGSGGGGSQTVV |
| | | TQEPSLTVSPGGTVTLTCGSSTGAVTSGNYPNWVQQKPGQAPRGLIGGTKFLAPGTP |
| | | ARFSGSLLGGKAALTLSGVQPEDEAEYYCVLWYSNRWVEGGGTKLTVLHHHHHH |
| SEQ ID | 3G1 TriTAC | QVRLVESGGGLVQAGESLRLSCAASGRPFSINTMGWYRQAPGKQRELVASISSSGDF |
| NO: 64 | | TYTDSVKGRFTISRDNAKNTVYLQMNSLKPEDTAVYYCNARRTYLPRRFGSWGQGTQ |
| | | VTVSSGGGGSGGGSEVQLVESGGGLVQPGNSLRLSCAASGFTFSSFGMSWVRQAPGK |
| | | GLEWVSSISGSGSDTLYADSVKGRFTISRDNAKTTLYLQMNSLRPEDTAVYYCTIGG |
| | | SLSRSSQGTLVTVSSGGGGSGGGSEVQLVESGGGLVQPGGSLKLSCAASGFTFNKYA |
| | | MNWVRQAPGKGLEWVARIRSKYNNYATYYADSVKDRFTISRDDSKNTAYLQMNNLKT |
| | | EDTAVYYCVRHGNEGNSYISYWAYWGQGTLVTVSSGGGGSGGGGSGGGGSQTVVTQE |
| | | PSLTVSPGGTVTLTCGSSTGAVTSGNYPNWVQQKPGQAPRGLIGGTKFLAPGTPARF |
| | | SGSLLGGKAALTLSGVQPEDEAEYYCVLWYSNRWVEGGGTKLTVLHHHHHH |
| SEQ ID | 2A1 TriTAC | QVQPVESGGGLVQPGGSLRLSCVVSGSDFTEDAMAWYRQASGKERESVAFVSKDGKR |
| NO: 65 | | ILYLDSVRGRFTISRDIDKKTVYLQMDNLKPEDTGVYYCNSAPGAARNYWGQGTQVT |
| | | VSSGGGGSGGGSEVQLVESGGGLVQPGNSLRLSCAASGFTFSSFGMSWVRQAPGKGL |
| | | EWVSSISGSGSDTLYADSVKGRFTISRDNAKTTLYLQMNSLRPEDTAVYYCTIGGSL |
| | | SRSSQGTLVTVSSGGGGSGGGSEVQLVESGGGLVQPGGSLKLSCAASGFTFNKYAMN |
| | | WVRQAPGKGLEWVARIRSKYNNYATYYADSVKDRFTISRDDSKNTAYLQMNNLKTED |
| | | TAVYYCVRHGNEGNSYISYWAYWGQGTLVTVSSGGGGSGGGGSGGGGSQTVVTQEPS |
| | | LTVSPGGTVTLTCGSSTGAVTSGNYPNWVQQKPGQAPRGLIGGTKFLAPGTPARFSG |
| | | SLLGGKAALTLSGVQPEDEAEYYCVLWYSNRWVFGGGTKLTVLHHHHHH |
| SEQ ID | 6F3 TriTAC | QVQPVESGGGLVQPGGSLRLSCVVSGSDFTEDAMAWYRQASGKERESVAFVSKDGKR |
| NO: 66 | | ILYLDSVRGRFTISRDIYKKTVYLQMDNLKPEDTGVYYCNSAPGAARNVWGQGTQVT |
| | | VSSGGGGSGGGSEVQLVESGGGLVQPGNSLRLSCAASGFTFSSFGMSWVRQAPGKGL |
| | | EWVSSISGSGSDTLYADSVKGRFTISRDNAKTTLYLQMNSLRPEDTAVYYCTIGGSL |
| | | SRSSQGTLVTVSSGGGGSGGGSEVQLVESGGGLVQPGGSLKLSCAASGFTFNKYAMN |
| | | WVRQAPGKGLEWVARIRSKYNNYATYYADSVKDRFTISRDDSKNTAYLQMNNLKTED |
| | | TAVYYCVRHGNEGNSYISYWAYWGQGTLVTVSSGGGGSGGGGSGGGGSQTVVTQEPS |
| | | LTVSPGGTVTLTCGSSTGAVTSGNYPNWVQQKPGQAPRGLIGGTKFLAPGTPARFSG |
| | | SLLGGKAALTLSGVQPEDEAEYYCVLWYSNRWVFGGGTKLTVLHHHHHH |
| SEQ ID | 1H2 TriTAC | EVQLVESGGGLVQPGNSLRLSCAASGFTFSSFGMSWVRQAPGKGLEWVSSISGSGSD |
| NO: 67 | | TLYADSVKGRFTISRDNAKTTLYLQMNSLRPEDTAVYYCTIGGSLSRSSQGTLVTVS |
| | | SGGGGSGGGSEVQLVESGGGLVQPGNSLRLSCAASGFTFSSFGMSWVRQAPGKGLEW |
| | | VSSISGSGSDTLYADSVKGRFTISRDNAKTTLYLQMNSLRPEDTAVYYCTIGGSLSR |
| | | SSQGTLVTVSSGGGGSGGGSEVQLVESGGGLVQPGGSLKLSCAASGFTFNKYAMNWV |
| | | RQAPGKGLEWVARIRSKYNNYATYYADSVKDRFTISRDDSKNTAYLQMNNLKTEDTA |
| | | VYYCVRHGNEGNSYISYWAYWGQGTLVTVSSGGGGSGGGGSGGGGSQTVVTQEPSLT |
| | | VSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLHHHHHH |
| SEQ ID | 3F2 TriTAC | QVQIVESGGGLVQAGGSLRLSCVASGLTYSIVAVGWYRQAPGKEREMVADISPVGNT |
| NO: 68 | | NYADSVKGRFTISKENAKNTVYLQMNSLKPEDTAVYYCHIVRGWLDERPGPGPIVYW |
| | | GQGTQVTVSSGGGGSGGGSEVQLVESGGGLVQPGNSLRLSCAASGFTFSSFGMSWVR |
| | | QAPGKGLEWVSSISGSGSDTLYADSVKGRFTISRDNAKTTLYLQMNSLRPEDTAVYY |
| | | CTIGGSLSRSSQGTLVTVSSGGGGSGGGSEVQLVESGGGLVQPGGSLKLSCAASGFT |
| | | FNKYAMNWVRQAPGKGLEWVARIRSKYNNYATYYADSVKDRFTISRDDSKNTAYLQM |
| | | NNLKTEDTAVYYCVRHGNFGNSYISYWAYWGQGTLVTVSSGGGGSGGGGSGGGGSQT |
| | | VVTQEPSLTVSPGGTVTLTCGSSTGAVTSGNYPNWVQQKPGQAPRGLIGGTKFLAPG |
| | | TPARFSGSLLGGKAALTLSGVQPEDEAEYYCVLWYSNRWVEGGGTKLTVLHHHHHH |
| SEQ ID | 12C2 TriTAC | QVQLVESGGGLVQTGGSLRLSCAASGLTFGVYGMEWFRQAPGKQREWVASHTSTGYV |
| NO: 69 | | YYRDSVKGRFTISRDNAKSTVYLQMNSLKPEDTAIYYCKANRGSYEYWGQGTQVTVS |
| | | SGGGGSGGGSEVQLVESGGGLVQPGNSLRLSCAASGFTFSSFGMSWVRQAPGKGLEW |
| | | VSSISGSGSDTLYADSVKGRFTISRDNAKTTLYLQMNSLRPEDTAVYYCTIGGSLSR |
| | | SSQGTLVTVSSGGGGSGGGSEVQLVESGGGLVQPGGSLKLSCAASGFTFNKYAMNWV |
| | | RQAPGKGLEWVARIRSKYNNYATYYADSVKDRFTISRDDSKNTAYLQMNNLKTEDTA |
| | | VYYCVRHGNEGNSYISYWAYWGQGTLVTVSSGGGGSGGGGSGGGGSQTVVTQEPSLT |
| | | VSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLHHHHHH |
| SEQ ID | 2D1 TriTAC | QVQLVESGGGLVQAGGSLRLSCAASTTSSINSMSWYRQAQGKQREPVAVITDRGSTS |
| NO: 70 | | YADSVKGRFTISRDNAKNTVYLQMNSLKPEDTAIYTCHVIADWRGYWGQGTQVTVSS |
| | | GGGGSGGGSEVQLVESGGGLVQPGNSLRLSCAASGFTFSSFGMSWVRQAPGKGLEWV |
| | | SSISGSGSDTLYADSVKGRFTISRDNAKTTLYLQMNSLRPEDTAVYYCTIGGSLSRS |
| | | SQGTLVTVSSGGGGSGGGSEVQLVESGGGLVQPGGSLKLSCAASGFTFNKYAMNWVR |
| | | QAPGKGLEWVARIRSKYNNYATYYADSVKDRFTISRDDSKNTAYLQMNNLKTEDTAV |
| | | YYCVRHGNEGNSYISYWAYWGQGTLVTVSSGGGGSGGGGSGGGGSQTVVTQEPSLTV |
| | | SPGGTVTLTCGSSTGAVTSGNYPNWVQQKPGQAPRGLIGGTKFLAPGTPARFSGSLL |
| | | GGKAALTLSGVQPEDEAEYYCVLWYSNRWVFGGGTKLTVLHHHHHH |
| SEQ ID | 6H2 TriTAC | QVQLVESGGGLVQAGGSLRLSCAASGRTLSRYAMGWFRQAPGKERQFVAAISRSGGT |
| NO: 71 | | TRYSDSVKGRFTISRDNAANTFYLQMNNLRPDDTAVYYCNVRRRGWGRTLEYWGQGT |
| | | QVTVSSGGGGSGGGSEVQLVESGGGLVQPGNSLRLSCAASGFTFSSFGMSWVRQAPG |
| | | KGLEWVSSISGSGSDTLYADSVKGRFTISRDNAKTTLYLQMNSLRPEDTAVYYCTIG |
| | | GSLSRSSQGTLVTVSSGGGGSGGGSEVQLVESGGGLVQPGGSLKLSCAASGFTFNKY |
| | | AMNWVRQAPGKGLEWVARIRSKYNNYATYYADSVKDRFTISRDDSKNTAYLQMNNLK |
| | | TEDTAVYYCVRHGNEGNSYISYWAYWGQGTLVTVSSGGGGSGGGGSGGGGSQTVVTQ |
| | | EPSLTVSPGGTVTLTCGSSTGAVTSGNYPNWVQQKPGQAPRGLIGGTKFLAPGTPAR |
| | | FSGSLLGGKAALTLSGVQPEDEAEYYCVLWYSNRWVEGGGTKLTVLHHHHHH |
| SEQ ID | 5D2 TriTAC | QVQLGESGGGLVQAGGSLRLSCAASGSIFSPNAMIWHRQAPGKQREPVASINSSGST |
| NO: 72 | | NYGDSVKGRFTVSRDIVKNTMYLQMNSLKPEDTAVYYCSYSDFRRGTQYWGQGTQVT |
| | | VSSGGGGSGGGSEVQLVESGGGLVQPGNSLRLSCAASGFTFSSFGMSWVRQAPGKGL |
| | | EWVSSISGSGSDTLYADSVKGRFTISRDNAKTTLYLQMNSLRPEDTAVYYCTIGGSL |
| | | SRSSQGTLVTVSSGGGGSGGGSEVQLVESGGGLVQPGGSLKLSCAASGFTFNKYAMN |
| | | WVRQAPGKGLEWVARIRSKYNNYATYYADSVKDRFTISRDDSKNTAYLQMNNLKTED |
| | | TAVYYCVRHGNEGNSYISYWAYWGQGTLVTVSSGGGGSGGGGSGGGGSQTVVTQEPS |
| | | LTVSPGGTVTLTCGSSTGAVTSGNYPNWVQQKPGQAPRGLIGGTKFLAPGTPARFSG |
| | | SLLGGKAALTLSGVQPEDEAEYYCVLWYSNRWVFGGGTKLTVLHHHHHH |
| SEQ ID | 7C4 TriTAC | QVQLVESGGGLVPSGGSLRLSCAASGATSAITNLGWYRRAPGQVREMVARISVREDK |
| NO: 73 | | EDYEDSVKGRFTISRDNTQNLVYLQMNNLQPHDTAIYYCGAQRWGRGPGTTWGQGTQ |
| | | VTVSSGGGGSGGGSEVQLVESGGGLVQPGNSLRLSCAASGFTFSSFGMSWVRQAPGK |
| | | GLEWVSSISGSGSDTLYADSVKGRFTISRDNAKTTLYLQMNSLRPEDTAVYYCTIGG |
| | | SLSRSSQGTLVTVSSGGGGSGGGSEVQLVESGGGLVQPGGSLKLSCAASGFTFNKYA |
| | | MNWVRQAPGKGLEWVARIRSKYNNYATYYADSVKDRFTISRDDSKNTAYLQMNNLKT |
| | | EDTAVYYCVRHGNEGNSYISYWAYWGQGTLVTVSSGGGGSGGGGSGGGGSQTVVTQE |
| | | PSLTVSPGGTVTLTCGSSTGAVTSGNYPNWVQQKPGQAPRGLIGGTKFLAPGTPARF |
| | | SGSLLGGKAALTLSGVQPEDEAEYYCVLWYSNRWVEGGGTKLTVLHHHHHH |
| SEQ ID | 5F2 TriTAC | QVQLVESGGGLVQAGGSLRLSCAASGSTFRIRVMRWYRQAPGTERDLVAVISGSSTY |
| NO: 74 | | YADSVKGRFTISRDNAKNTLYLQMNNLKPEDTAVYYCNADDSGIARDYWGQGTQVTV |
| | | SSGGGGSGGGSEVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVYYCTIGGSLS |
| | | RSSQGTLVTVSSGGGGSGGGSEVQLVESGGGLVQPGGSLKLSCAASGFTFNKYAMNW |
| | | VRQAPGKGLEWVARIRSKYNNYATYYADSVKDRFTISRDDSKNTAYLQMNNLKTEDT |
| | | AVYYCVRHGNEGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGS |
| | | LLGGKAALTLSGVQPEDEAEYYCVLWYSNRWVFGGGTKLTVLHHHHHH |
| SEQ ID | 2C2 TriTAC | QVQLVESGGGLVQAGESRRLSCAVSGDTSKFKAVGWYRQAPGAQRELLAWINNSGVG |
| NO: 75 | | NTAESVKGRFTISRDNAKNTVYLQMNRLTPEDTDVYYCRFYRREGINKNYWGQGTQV |
| | | TVSSGGGGSGGGSEVQLVESGGGLVQPGNSLRLSCAASGFTFSSFGMSWVRQAPGKG |
| | | LEWVSSISGSGSDTLYADSVKGRFTISRDNAKTTLYLQMNSLRPEDTAVYYCTIGGS |
| | | LSRSSQGTLVTVSSGGGGSGGGSEVQLVESGGGLVQPGGSLKLSCAASGFTFNKYAM |
| | | NWVRQAPGKGLEWVARIRSKYNNYATYYADSVKDRFTISRDDSKNTAYLQMNNLKTE |
| | | DTAVYYCVRHGNEGNSYISYWAYWGQGTLVTVSSGGGGSGGGGSGGGGSQTVVTQEP |
| | | SLTVSPGGTVTLTCGSSTGAVTSGNYPNWVQQKPGQAPRGLIGGTKFLAPGTPARFS |
| | | GSLLGGKAALTLSGVQPEDEAEYYCVLWYSNRWVEGGGTKLTVLHHHHHH |
| SEQ ID | 5G2 TriTAC | QVQLVESGGGLVQAGGSLRLSCAASGSTFGNKPMGWYRQAPGKQRELVAVISSDGGS |
| NO: 76 | | TRYAALVKGRFTISRDNAKNTVYLQMESLVAEDTAVYYCNALRTYYLNDPVVESWGQ |
| | | GTQVTVSSGGGGSGGGSEVQLVESGGGLVQPGNSLRLSCAASGFTFSSFGMSWVRQA |
| | | PGKGLEWVSSISGSGSDTLYADSVKGRFTISRDNAKTTLYLQMNSLRPEDTAVYYCT |
| | | IGGSLSRSSQGTLVTVSSGGGGSGGGSEVQLVESGGGLVQPGGSLKLSCAASGFTFN |
| | | KYAMNWVRQAPGKGLEWVARIRSKYNNYATYYADSVKDRFTISRDDSKNTAYLQMNN |
| | | LKTEDTAVYYCVRHGNEGNSYISYWAYWGQGTLVTVSSGGGGSGGGGSGGGGSQTVV |
| | | TQEPSLTVSPGGTVTLTCGSSTGAVTSGNYPNWVQQKPGQAPRGLIGGTKFLAPGTP |
| | | ARFSGSLLGGKAALTLSGVQPEDEAEYYCVLWYSNRWVEGGGTKLTVLHHHHHH |
| SEQ ID | 9H2 TriTAC | QVQLVESGGGLVQAGGSLRLSCAASGSTSSINTMYWYRQAPGKERELVAFISSGGST |
| NO: 77 | | NVRDSVKGRFSVSRDSAKNIVYLQMNSLTPEDTAVYYCNTYIPLRGTLHDYWGQGTQ |
| | | VTVSSGGGGSGGGSEVQLVESGGGLVQPGNSLRLSCAASGFTFSSFGMSWVRQAPGK |
| | | GLEWVSSISGSGSDTLYADSVKGRFTISRDNAKTTLYLQMNSLRPEDTAVYYCTIGG |
| | | SLSRSSQGTLVTVSSGGGGSGGGSEVQLVESGGGLVQPGGSLKLSCAASGFTFNKYA |
| | | MNWVRQAPGKGLEWVARIRSKYNNYATYYADSVKDRFTISRDDSKNTAYLQMNNLKT |
| | | EDTAVYYCVRHGNEGNSYISYWAYWGQGTLVTVSSGGGGSGGGGSGGGGSQTVVTQE |
| | | PSLTVSPGGTVTLTCGSSTGAVTSGNYPNWVQQKPGQAPRGLIGGTKFLAPGTPARF |
| | | SGSLLGGKAALTLSGVQPEDEAEYYCVLWYSNRWVEGGGTKLTVLHHHHHH |
| SEQ ID | 5D4 TriTAC | QVQLVESGGGLVQAGGSLRLSCVASGRTDRITTMGWYRQAPGKQRELVATISNRGTS |
| NO: 78 | | NYANSVKGRFTISRDNAKNTVYLQMNSLKPEDTAVYYCNARKWGRNYWGQGTQVTVS |
| | | SGGGGSGGGSEVQLVESGGGLVQPGNSLRLSCAASGFTFSSFGMSWVRQAPGKGLEW |
| | | VSSISGSGSDTLYADSVKGRFTISRDNAKTTLYLQMNSLRPEDTAVYYCTIGGSLSR |
| | | SSQGTLVTVSSGGGGSGGGSEVQLVESGGGLVQPGGSLKLSCAASGFTENKYAMNWV |
| | | RQAPGKGLEWVARIRSKYNNYATYYADSVKDRFTISRDDSKNTAYLQMNNLKTEDTA |
| | | VYYCVRHGNEGNSYISYWAYWGQGTLVTVSSGGGGSGGGGSGGGGSQTVVTQEPSLT |
| | | VSPGGTVTLTCGSSTGAVTSGNYPNWVQQKPGQAPRGLIGGTKFLAPGTPARFSGSL |
| | | LGGKAALTLSGVQPEDEAEYYCVLWYSNRWVFGGGTKLTVLHHHHHH |
| SEQ ID | 2A4 TriTAC | QVQLVESGGGLVQARGSLRLSCTASGRTIGINDMAWYRQAPGNQRELVATITKGGTT |
| NO: 79 | | DYADSVDGRFTISRDNAKNTVYLQMNSLKPEDTAVYYCNTKRREWAKDFEYWGQGTQ |
| | | VTVSSGGGGSGGGSEVQLVESGGGLVQPGNSLRLSCAASGFTFSSFGMSWVRQAPGK |
| | | GLEWVSSISGSGSDTLYADSVKGRFTISRDNAKTTLYLQMNSLRPEDTAVYYCTIGG |
| | | SLSRSSQGTLVTVSSGGGGSGGGSEVQLVESGGGLVQPGGSLKLSCAASGFTFNKYA |
| | | MNWVRQAPGKGLEWVARIRSKYNNYATYYADSVKDRFTISRDDSKNTAYLQMNNLKT |
| | | EDTAVYYCVRHGNEGNSYISYWAYWGQGTLVTVSSGGGGSGGGGSGGGGSQTVVTQE |
| | | PSLTVSPGGTVTLTCGSSTGAVTSGNYPNWVQQKPGQAPRGLIGGTKFLAPGTPARF |
| | | SGSLLGGKAALTLSGVQPEDEAEYYCVLWYSNRWVEGGGTKLTVLHHHHHH |
| SEQ ID | 7F1 TriTAC | QVQLVESGGGLVQAGGSLRLSCAASAIGSINSMSWYRQAPGKQREPVAVITDRGSTS |
| NO: 80 | | YADSVKGRFTISRDNAKNTVYLQMNSLKPEDTAIYTCHVIADWRGYWGQGTQVTVSS |
| | | GGGGSGGGSEVQLVESGGGLVQPGNSLRLSCAASGFTFSSFGMSWVRQAPGKGLEWV |
| | | SSISGSGSDTLYADSVKGRFTISRDNAKTTLYLQMNSLRPEDTAVYYCTIGGSLSRS |
| | | SQGTLVTVSSGGGGSGGGSEVQLVESGGGLVQPGGSLKLSCAASGFTENKYAMNWVR |
| | | QAPGKGLEWVARIRSKYNNYATYYADSVKDRFTISRDDSKNTAYLQMNNLKTEDTAV |
| | | YYCVRHGNEGNSYISYWAYWGQGTLVTVSSGGGGSGGGGSGGGGSQTVVTQEPSLTV |
| | | SPGGTVTLTCGSSTGAVTSGNYPNWVQQKPGQAPRGLIGGTKFLAPGTPARFSGSLL |
| | | GGKAALTLSGVQPEDEAEYYCVLWYSNRWVFGGGTKLTVLHHHHHH |
| SEQ ID | 5C2 TriTAC | QVQLVESGGGLVQAGGSLRLSCAASGSTSSINTMYWFRQAPGEERELVATINRGGST |
| NO: 81 | | NVRDSVKGRFSVSRDSAKNIVYLQMNRLKPEDTAVYYCNTYIPYGGTLHDFWGQGTQ |
| | | VTVSSGGGGSGGGSEVQLVESGGGLVQPGNSLRLSCAASGFTFSSFGMSWVRQAPGK |
| | | GLEWVSSISGSGSDTLYADSVKGRFTISRDNAKTTLYLQMNSLRPEDTAVYYCTIGG |
| | | SLSRSSQGTLVTVSSGGGGSGGGSEVQLVESGGGLVQPGGSLKLSCAASGFTFNKYA |
| | | MNWVRQAPGKGLEWVARIRSKYNNYATYYADSVKDRFTISRDDSKNTAYLQMNNLKT |
| | | EDTAVYYCVRHGNEGNSYISYWAYWGQGTLVTVSSGGGGSGGGGSGGGGSQTVVTQE |
| | | PSLTVSPGGTVTLTCGSSTGAVTSGNYPNWVQQKPGQAPRGLIGGTKFLAPGTPARF |
| | | SGSLLGGKAALTLSGVQPEDEAEYYCVLWYSNRWVEGGGTKLTVLHHHHHH |
| SEQ ID | 2F4 TriTAC | QVQLVESGGGLVQAGGSLRLSCTTSTTFSINSMSWYRQAPGNQREPVAVITNRGTTS |
| NO: 82 | | YADSVKGRFTISRDNARNTVYLQMDSLKPEDTAIYTCHVIADWRGYWGQGTQVTVSS |
| | | GGGGSGGGSEVQLVESGGGLVQPGNSLRLSCAASGFTFSSFGMSWVRQAPGKGLEWV |
| | | SSISGSGSDTLYADSVKGRFTISRDNAKTTLYLQMNSLRPEDTAVYYCTIGGSLSRS |
| | | SQGTLVTVSSGGGGSGGGSEVQLVESGGGLVQPGGSLKLSCAASGFTENKYAMNWVR |
| | | QAPGKGLEWVARIRSKYNNYATYYADSVKDRFTISRDDSKNTAYLQMNNLKTEDTAV |
| | | YYCVRHGNEGNSYISYWAYWGQGTLVTVSSGGGGSGGGGSGGGGSQTVVTQEPSLTV |
| | | SPGGTVTLTCGSSTGAVTSGNYPNWVQQKPGQAPRGLIGGTKFLAPGTPARFSGSLL |
| | | GGKAALTLSGVQPEDEAEYYCVLWYSNRWVFGGGTKLTVLHHHHHH |
| SEQ ID | 2A2 TriTAC | QVQLVESGGGLVQAGGSLTLSCAASGSTFSIRAMRWYRQAPGTERDLVAVIYGSSTY |
| NO: 83 | | YADAVKGRFTISRDNAKNTLYLQMNNLKPEDTAVYYCNADTIGTARDYWGQGTQVTV |
| | | SSGGGGSGGGSEVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVYYCTIGGSLS |
| | | RSSQGTLVTVSSGGGGSGGGSEVQLVESGGGLVQPGGSLKLSCAASGFTENKYAMNW |
| | | VRQAPGKGLEWVARIRSKYNNYATYYADSVKDRFTISRDDSKNTAYLQMNNLKTEDT |
| | | AVYYCVRHGNEGNSYISYWAYWGQGTLVTVSSGGGGSGGGGSGGGGSQTVVTQEPSL |
| | | TVSPGGTVTLTCGSSTGAVTSGNYPNWVQQKPGQAPRGLIGGTKFLAPGTPARFSGS |
| | | LLGGKAALTLSGVQPEDEAEYYCVLWYSNRWVFGGGTKLTVLHHHHHH |
| SEQ ID | 11F3 TriTAC | QVQLVESGGGLVQAGGSLRLSCVASGRTSTIDTMYWHRQAPGNERELVAYVTSRGTS |
| NO: 84 | | NVADSVKGRFTISRDNAKNTAYLQMNSLKPEDTAVYYCSVRTTSYPVDFWGQGTQVT |
| | | VSSGGGGSGGGSEVQLVESGGGLVQPGNSLRLSCAASGFTFSSFGMSWVRQAPGKGL |
| | | EWVSSISGSGSDTLYADSVKGRFTISRDNAKTTLYLQMNSLRPEDTAVYYCTIGGSL |
| | | SRSSQGTLVTVSSGGGGSGGGSEVQLVESGGGLVQPGGSLKLSCAASGFTENKYAMN |
| | | WVRQAPGKGLEWVARIRSKYNNYATYYADSVKDRFTISRDDSKNTAYLQMNNLKTED |
| | | TAVYYCVRHGNEGNSYISYWAYWGQGTLVTVSSGGGGSGGGGSGGGGSQTVVTQEPS |
| | | LTVSPGGTVTLTCGSSTGAVTSGNYPNWVQQKPGQAPRGLIGGTKFLAPGTPARFSG |
| | | SLLGGKAALTLSGVQPEDEAEYYCVLWYSNRWVFGGGTKLTVLHHHHHH |
| SEQ ID | 10B3 TriTAC | QVQLVESGGGLVQAGGSLRLSCAASGSTSSINTMYWYRQAPGKERELVAFISSGGST |
| NO: 85 | | NVRDSVKGRFSVSRDSAKNIVYLQMNSLKPEDTAVYYCNTYIPYGGTLHDFWGQGTQ |
| | | VTVSSGGGGSGGGSEVQLVESGGGLVQPGNSLRLSCAASGFTFSSFGMSWVRQAPGK |
| | | GLEWVSSISGSGSDTLYADSVKGRFTISRDNAKTTLYLQMNSLRPEDTAVYYCTIGG |
| | | SLSRSSQGTLVTVSSGGGGSGGGSEVQLVESGGGLVQPGGSLKLSCAASGFTFNKYA |
| | | MNWVRQAPGKGLEWVARIRSKYNNYATYYADSVKDRFTISRDDSKNTAYLQMNNLKT |
| | | EDTAVYYCVRHGNEGNSYISYWAYWGQGTLVTVSSGGGGSGGGGSGGGGSQTVVTQE |
| | | PSLTVSPGGTVTLTCGSSTGAVTSGNYPNWVQQKPGQAPRGLIGGTKFLAPGTPARF |
| | | SGSLLGGKAALTLSGVQPEDEAEYYCVLWYSNRWVEGGGTKLTVLHHHHHH |
| SEQ ID | 5H1 TriTAC | QVQLVESGGGLVQPGGSLRLSCAASGGDWSANFMYWYRQAPGKQRELVARISGRGVV |
| NO: 86 | | DYVESVKGRFTISRDNAKNTVYLQMNSLKPEDTAVYYCAVASYWGQGTQVTVSSGGG |
| | | GSGGGSEVQLVESGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYCTIGGSLSRSSQG |
| | | TLVTVSSGGGGSGGGSEVQLVESGGGLVQPGGSLKLSCAASGFTENKYAMNWVRQAP |
| | | GKGLEWVARIRSKYNNYATYYADSVKDRFTISRDDSKNTAYLQMNNLKTEDTAVYYC |
| | | VRHGNEGNSYISYWAYWGQGTLVTVSSGGGGSGGGGSGGGGSQTVVTQEPSLTVSPG |
| | | GTVTLTCGSSTGAVTSGNYPNWVQQKPGQAPRGLIGGTKFLAPGTPARFSGSLLGGK |
| | | AALTLSGVQPEDEAEYYCVLWYSNRWVFGGGTKLTVLHHHHHH |
| SEQ ID | Exemplary | (GS)n |
| NO: 87 | linker | |
| | sequence | |
| SEQ ID | Exemplary | (GGS)n |
| NO: 88 | linker | |
| | sequence | |
| SEQ ID | Exemplary | (GGGS)n |
| NO: 89 | linker | |
| | sequence | |
| SEQ ID | Exemplary | (GGSG)n |
| NO: 90 | linker | |
| | sequence | |
| SEQ ID | Exemplary | (GGSGG)n |
| NO: 91 | linker | |
| | sequence | |
| SEQ ID | Exemplary | (GGGGS)n |
| NO: 92 | linker | |
| | sequence | |
| SEQ ID | Exemplary | (GGGGG)n |
| NO: 93 | linker | |
| | sequence | |
| SEQ ID | Exemplary | (GGG)n |
| NO: 94 | linker | |
| | sequence | |
| SEQ ID | Exemplary | (GGGGS)4 |
| NO: 95 | linker | |
| | sequence | |
| SEQ ID | Exemplary | (GGGGS)3 |
| NO: 96 | linker | |
| | sequence | |
| SEQ ID | Sortase | LPETG |
| NO: 97 | recognition | |
| | domain | |
| SEQ ID | MH6T TriTAC | QVQLVESGGGVVQAGGSLTLSCAASGSTESIRAMRWYRQAPGTERDLVAVIYGSSTY |
| NO: 98 | | YADAVKGRFTISRDNSKNTLYLQMNSLRAEDTAVYYCNADTIGTARDYWGQGTLVTV |
| | | SSGGGGSGGGSEVQLVESGGGLVQPGNSLRLSCAASGFTFSKFGMSWVRQAPGKGLE |
| | | WVSSISGSGRDTLYADSVKGRFTISRDNAKTTLYLQMNSLRPEDTAVYYCTIGGSLS |
| | | VSSQGTLVTVSSGGGGSGGGSEVQLVESGGGLVQPGGSLKLSCAASGFTFNKYAINW |
| | | VRQAPGKGLEWVARIRSKYNNYATYYADQVKDRFTISRDDSKNTAYLQMNNLKTEDT |
| | | AVYYCVRHANEGNSYISYWAYWGQGTLVTVSSGGGGSGGGGSGGGGSQTVVTQEPSL |
| | | TVSPGGTVTLTCASSTGAVTSGNYPNWVQQKPGQAPRGLIGGTKFLVPGTPARFSGS |
| | | LLGGKAALTLSGVQPEDEAEYYCTLWYSNRWVFGGGTKLTVLHHHHHH |
| SEQ ID | GFP TriTAC | QVQLVESGGALVQPGGSLRLSCAASGFPVNRYSMRWYRQAPGKEREWVAGMSSAGDR |
| NO: 99 | | SSYEDSVKGRFTISRDDARNTVYLQMNSLKPEDTAVYYCNVNVGFEYWGQGTQVTVS |
| | | SGGGGSGGGSEVQLVESGGGLVQPGNSLRLSCAASGFTFSKFGMSWVRQAPGKGLEW |
| | | VSSISGSGRDTLYADSVKGRFTISRDNAKTTLYLQMNSLRPEDTAVYYCTIGGSLSV |
| | | SSQGTLVTVSSGGGGSGGGSEVQLVESGGGLVQPGGSLKLSCAASGFTFNKYAINWV |
| | | RQAPGKGLEWVARIRSKYNNYATYYADQVKDRFTISRDDSKNTAYLQMNNLKTEDTA |
| | | VYYCVRHANEGNSYISYWAYWGQGTLVTVSSGGGGSGGGGSGGGGSQTVVTQEPSLT |
| | | VSPGGTVTLTCASSTGAVTSGNYPNWVQQKPGQAPRGLIGGTKFLVPGTPARFSGSL |
| | | LGGKAALTLSGVQPEDEAEYYCTLWYSNRWVFGGGTKLTVLHHHHHH |
| SEQ ID | TriTAC 74 | QVQLVESGGGVVQAGGSLRLSCAASGSTFSIRAMRWYRQAPGTERDLVAVIYGSSTY |
| NO: 100 | | YADAVKGRFTISRDNSKNTLYLQMNSLRAEDTAVYYCNADTIGTARDYWGQGTLVTV |
| | | SSGGGGSGGGSEVQLVESGGGLVQPGNSLRLSCAASGFTFSKFGMSWVRQAPGKGLE |
| | | WVSSISGSGRDTLYADSVKGRFTISRDNAKTTLYLQMNSLRPEDTAVYYCTIGGSLS |
| | | VSSQGTLVTVSSGGGGSGGGSEVQLVESGGGLVQPGGSLKLSCAASGNTFNKYAMNW |
| | | VRQAPGKGLEWVARIRSKYNNYATYYADSVKDRFTISRDDSKNTAYLQMNNLKTEDT |
| | | AVYYCVRHGNFGDSYISYWAYWGQGTLVTVSSGGGGSGGGGSGGGGSQTVVTQEPSL |
| | | TVSPGGTVTLTCGSSTGAVTHGNYPNWVQQKPGQAPRGLIGGTKVLAPGTPARFSGS |
| | | LLGGKAALTLSGVQPEDEAEYYCVLWYSNRWVFGGGTKLTVLHHHHHH |
| SEQ ID | TriTAC 75 | QVQLVESGGGVVQAGGSLRLSCAASGSTESIRAMRWYRQAPGTERDLVAVIYGSSTY |
| NO: 101 | | YADAVKGRFTISRDNSKNTLYLQMNSLRAEDTAVYYCNADTIGTARDYWGQGTLVTV |
| | | SSGGGGSGGGSEVQLVESGGGLVQPGNSLRLSCAASGFTFSKFGMSWVRQAPGKGLE |
| | | WVSSISGSGRDTLYADSVKGRFTISRDNAKTTLYLQMNSLRPEDTAVYYCTIGGSLS |
| | | VSSQGTLVTVSSGGGGSGGGSEVQLVESGGGLVQPGGSLKLSCAASGFTFNKYAINW |
| | | VRQAPGKGLEWVARIRSKYNNYATYYADQVKDRFTISRDDSKNTAYLQMNNLKTEDT |
| | | AVYYCVRHANEGNSYISYWAYWGQGTLVTVSSGGGGSGGGGSGGGGSQTVVTQEPSL |
| | | TVSPGGTVTLTCASSTGAVTSGNYPNWVQQKPGQAPRGLIGGTKFLVPGTPARFSGS |
| | | LLGGKAALTLSGVQPEDEAEYYCTLWYSNRWVFGGGTKLTVLHHHHHH |
| SEQ ID | Anti-MSLN- | QVQLVESGGGVVQAGGSLTLSCAASGSTESIRAMRWYRQAPGTERDLVAVIYGSSTY |
| NO: 102 | MH6T | YADAVKGRFTISRDNSKNTLYLQMNSLRAEDTAVYYCNADTIGTARDYWGQGTLVTV |
| | | SS |
| SEQ ID | MH6 | QVQLVESGGGVVQAGGSLRLSCAASGSTESIRAMRWYRQAPGTERDLVAVIYGSSTY |
| NO: 103 | (exemplary | YADAVKGRFTISRDNSKNTLYLQMNSLRAEDTAVYYCNADTIGTARDYWGQGTLVTV |
| | humanized | SS |
| | version of | |
| | 2A2) | |
| SEQ ID | MH7 | QVQLVESGGGVVQPGGSLRLSCAASGSTESIRAMRWYRQAPGKERELVAVIYGSSTY |
| NO: 104 | (exemplary | YADAVKGRFTISRDNSKNTLYLQMNSLRAEDTAVYYCNADTIGTARDYWGQGTLVTV |
| | humanized | SS |
| | version of | |
| | 2A2) | |
| SEQ ID | MH8 | QVQLVESGGGVVQPGGSLRLSCAASGSTESIRAMRWVRQAPGKGLEWVSVIYGSSTY |
| NO: 105 | (exemplary | YADAVKGRFTISRDNSKNTLYLQMNSLRAEDTAVYYCNADTIGTARDYWGQGTLVTV |
| | humanized | SS |
| | version of | |
| | 2A2) | |

Sequence Table for CDRs of exemplary Mesothelin
binding trispecific proteins of this disclosure

| | Exemplary MSLN | |
|---|---|---|
| | binding | |
| Sequence | trispecific | |
| ID No. | protein/TriTAC | CDR1 Sequence |
| 106 | 9B1 | GRTFSVRGMA |
| 107 | 9F3 | GSIPSIEQMG |
| 108 | 7H2 | GTTYTFDLMS |
| 109 | 3B4 | GSTSNINNMR |
| 110 | 4A2 | GSTFGINAMG |
| 111 | 12D1 | ISAFRLMSVR |
| 112 | 3G1 | GRPFSINTMG |
| 113 | 2A1 | GSDFTEDAMA |
| 114 | 6F3 | GSDFTEDAMA |
| 115 | 1H2 | GFTFSSFGMS |
| 116 | 3F2 | GLTYSIVAVG |
| 117 | 12C2 | GLTFGVYGME |
| 118 | 2D1 | TTSSINSMS |
| 119 | 6H2 | GRTLSRYAMG |
| 120 | 5D2 | GSIFSPNAMI |
| 121 | 7C4 | GATSAITNLG |
| 122 | 5F2 | GSTFRIRVMR |
| 123 | 2C2 | GDTSKFKAVG |
| 124 | 5G2 | GSTFGNKPMG |
| 125 | 9H2 | GSTSSINTMY |
| 126 | 5D4 | GRTDRITTMG |
| 127 | 2A4 | GRTIGINDMA |
| 128 | 7F1 | AIGSINSMS |
| 129 | 5C2 | GSTSSINTMY |
| 130 | 2F4 | TTFSINSMS |
| 131 | 2A2 | GSTFSIRAMR |
| 132 | 11F3 | GRTSTIDTMY |
| 133 | 10B3 | GSTSSINTMY |
| 134 | MH1 | GGDWSANFMY |
| 135 | MH2 | GGDWSANFMY |
| 136 | MH3 | GSTSSINTMY |
| 137 | MH4 | GSTSSINTMY |
| 138 | MH5 | GSTSSINTMY |
| 139 | MH6 | GSTFSIRAMR |
| 140 | MH7 | GSTFSIRAMR |
| 141 | MH8 | GSTFSIRAMR |
| 142 | MH9 | GRTSTIDTMY |
| 143 | MH10 | GRTSTIDTMY |
| 144 | MH11 | GRTSTIDTMY |

Sequence Table for CDR2s of exemplary Mesothelin
binding trispecific proteins of this disclosure

| | Exemplary | |
|---|---|---|
| Sequence | trispecific | |
| ID No. | protein/TriTAC | CDR2 Sequence |
| 145 | 9B1 | TMNPDGFPNYADAVKGRFT |
| 146 | 9F3 | ALTSGGRANYADSVKGRFT |
| 147 | 7H2 | SISSDGRTSYADSVRGRFT |
| 148 | 3B4 | VITRGGYAIYLDAVKGRFT |
| 149 | 4A2 | VISRGGSTNYADSVKGRFT |
| 150 | 12D1 | TIDQLGRTNYADSVKGRFA |
| 151 | 3G1 | SISSSGDFTYTDSVKGRFT |
| 152 | 2A1 | FVSKDGKRILYLDSVRGRFT |
| 153 | 6F3 | FVSKDGKRILYLDSVRGRFT |
| 154 | 1H2 | SISGSGSDTLYADSVKGRFT |
| 155 | 3F2 | DISPVGNTNYADSVKGRFT |
| 156 | 12C2 | SHTSTGYVYYRDSVKGRFT |
| 157 | 2D1 | VITDRGSTSYADSVKGRFT |
| 158 | 6H2 | AISRSGGTTRYSDSVKGRFT |
| 159 | 5D2 | SINSSGSTNYGDSVKGRFT |
| 160 | 7C4 | RISVREDKEDYEDSVKGRFT |
| 161 | 5F2 | VISGSSTYYADSVKGRFT |
| 162 | 2C2 | WINNSGVGNTAESVKGRFT |
| 163 | 5G2 | VISSDGGSTRYAALVKGRFT |
| 164 | 9H2 | FISSGGSTNVRDSVKGRFS |
| 165 | 5D4 | TISNRGTSNYANSVKGRFT |
| 166 | 2A4 | TITKGGTTDYADSVDGRFT |
| 167 | 7F1 | VITDRGSTSYADSVKGRFT |
| 168 | 5C2 | TINRGGSTNVRDSVKGRFS |
| 169 | 2F4 | VITNRGTTSYADSVKGRFT |
| 170 | 2A2 | VIYGSSTYYADAVKGRFT |
| 171 | 11F3 | YVTSRGTSNVADSVKGRFT |
| 172 | 10B3 | FISSGGSTNVRDSVKGRFS |
| 173 | MH1 | RISGRGVVDYVESVKGRFT |
| 174 | MH2 | RISGRGVVDYVESVKGRFT |
| 175 | MH3 | FISSGGSTNVRDSVKGRFT |
| 176 | MH4 | FISSGGSTNVRDSVKGRFT |
| 177 | MH5 | FISSGGSTNVRDSVKGRFT |
| 178 | MH6 | VIYGSSTYYADAVKGRFT |
| 179 | MH7 | VIYGSSTYYADAVKGRFT |
| 180 | MH8 | VIYGSSTYYADAVKGRFT |
| 181 | MH9 | YVTSRGTSNVADSVKGRFT |
| 182 | MH10 | YVTSRGTSNVADSVKGRFT |
| 183 | MH11 | YVTSRGTSNVADSVKGRFT |

Sequence Table for CDR3s of exemplary Mesothelin
binding trispecific proteins of this disclosure

| | Exemplary | |
|---|---|---|
| Sequence | trispecific | |
| ID No. | protein/TriTAC | CDR3 Sequence |
| 184 | 9B1 | GPY |
| 185 | 9F3 | GRFKGDYAQRSGMDY |
| 186 | 7H2 | QRSGVRAF |
| 187 | 3B4 | DRVEGTSGGPQLRDY |
| 188 | 4A2 | RTYTRHDY |
| 189 | 12D1 | GGGPLGSRWLRGRH |
| 190 | 3G1 | RRTYLPRRFGS |
| 191 | 2A1 | APGAARNY |
| 192 | 6F3 | APGAARNV |
| 193 | 1H2 | GGSLSRSS |
| 194 | 3F2 | VRGWLDERPGPGPIVY |
| 195 | 12C2 | NRGSYEY |
| 196 | 2D1 | IADWRGY |
| 197 | 6H2 | RRRGWGRTLEY |
| 198 | 5D2 | SDFRRGTQY |
| 199 | 7C4 | QRWGRGPGTT |
| 200 | 5F2 | DDSGIARDY |
| 201 | 2C2 | YRRFGINKNY |
| 202 | 5G2 | LRTYYLNDPVVFS |
| 203 | 9H2 | YIPLRGTLHDY |
| 204 | 5D4 | RKWGRNY |
| 205 | 2A4 | KRREWAKDFEY |
| 206 | 7F1 | IADWRGY |
| 207 | 5C2 | YIPYGGTLHDF |
| 208 | 2F4 | IADWRGY |
| 209 | 2A2 | DTIGTARDY |
| 210 | 11F3 | RTTSYPVDF |
| 211 | 10B3 | YIPYGGTLHDF |
| 212 | MH1 | ASY |
| 213 | MH2 | ASY |
| 214 | MH3 | YIPYGGTLHDF |
| 215 | MH4 | YIPYGGTLHDF |
| 216 | MH5 | YIPYGGTLHDF |
| 217 | MH6 | DTIGTARDY |
| 218 | MH7 | DTIGTARDY |
| 219 | MH8 | DTIGTARDY |
| 220 | MH9 | RTTSYPVDF |
| 221 | MH10 | RTTSYPVDF |
| 222 | MH11 | RTTSYPVDF |

Framework region 1 (fl) of exemplary MSLN
trispecific binding proteins of this disclosure

| | Exemplary | |
|---|---|---|
| | trispecific | |
| Sequence | protein/ | |
| ID No. | TriTAC | Framework 1 |
| 223 | 9B1 | QVQLVESGGGLVQPGGSLRLSCAAS |
| 224 | 9F3 | QVQLVESGGGLVQAGGSLRLSCAAS |
| 225 | 7H2 | QVQLVESGGGLVQAGGSLRLSCAFS |
| 226 | 3B4 | QVQLVESGGGLVQAGGSLRLSCVAS |
| 227 | 4A2 | QVQLVESGGGLVQAGGSLRLSCAAS |
| 228 | 12D1 | QVRLVESGGGLVQAGGSLRLSCAAS |
| 229 | 3G1 | QVRLVESGGGLVQAGESLRLSCAAS |
| 230 | 2A1 | QVQPVESGGGLVQPGGSLRLSCVVS |
| 231 | 6F3 | QVQPVESGGGLVQPGGSLRLSCVVS |
| 232 | 1H2 | EVQLVESGGGLVQPGNSLRLSCAAS |
| 233 | 3F2 | QVQIVESGGGLVQAGGSLRLSCVAS |
| 234 | 12C2 | QVQLVESGGGLVQTGGSLRLSCAAS |
| 235 | 2D1 | QVQLVESGGGLVQAGGSLRLSCAAS |
| 236 | 6H2 | QVQLVESGGGLVQAGGSLRLSCAAS |
| 237 | 5D2 | QVQLGESGGGLVQAGGSLRLSCAAS |
| 238 | 7C4 | QVQLVESGGGLVPSGGSLRLSCAAS |
| 239 | 5F2 | QVQLVESGGGLVQAGGSLRLSCAAS |
| 240 | 2C2 | QVQLVESGGGLVQAGESRRLSCAVS |
| 241 | 5G2 | QVQLVESGGGLVQAGGSLRLSCAAS |
| 242 | 9H2 | QVQLVESGGGLVQAGGSLRLSCAAS |
| 243 | 5D4 | QVQLVESGGGLVQAGGSLRLSCVAS |
| 244 | 2A4 | QVQLVESGGGLVQARGSLRLSCTAS |
| 245 | 7F1 | QVQLVESGGGLVQAGGSLRLSCAAS |
| 246 | 5C2 | QVQLVESGGGLVQAGGSLRLSCAAS |
| 247 | 2F4 | QVQLVESGGGLVQAGGSLRLSCTTS |
| 248 | 2A2 | QVQLVESGGGLVQAGGSLTLSCAAS |
| 249 | 11F3 | QVQLVESGGGLVQAGGSLRLSCVAS |
| 250 | 10B3 | QVQLVESGGGLVQAGGSLRLSCAAS |
| 251 | MH1 | EVQLVESGGGLVQPGGSLRLSCAAS |
| 252 | MH2 | EVQLVESGGGLVQPGGSLRLSCAAS |
| 253 | MH3 | EVQLVESGGGLVQAGGSLRLSCAAS |
| 254 | MH4 | EVQLVESGGGLVQPGGSLRLSCAAS |
| 255 | MH5 | EVQLVESGGGLVQPGGSLRLSCAAS |
| 256 | MH6 | QVQLVESGGGVVQAGGSLRLSCAAS |
| 257 | MH7 | QVQLVESGGGVVQPGGSLRLSCAAS |
| 258 | MH8 | QVQLVESGGGVVQPGGSLRLSCAAS |
| 259 | MH9 | EVQLVESGGGLVQAGGSLRLSCVAS |
| 260 | MH10 | EVQLVESGGGLVQPGGSLRLSCAAS |
| 261 | MH11 | EVQLVESGGGLVQPGGSLRLSCAAS |

Framework region 2 (f2) of exemplary MSLN
trispecific binding proteins of this disclosure

| | Exemplary | |
|---|---|---|
| Sequence | trispecific | |
| ID No. | protein/TriTAC | Framework 2 |
| 262 | 9B1 | WYRQAGNNRALVA |
| 263 | 9F3 | WYRQAPGKQRELVA |
| 264 | 7H2 | WYRQAPGKQRTVVA |
| 265 | 3B4 | WYRQAPGKERELVA |
| 266 | 4A2 | WYRQAPGKQRELVA |
| 267 | 12D1 | WYRQDPSKQREWVA |
| 268 | 3G1 | WYRQAPGKQRELVA |
| 269 | 2A1 | WYRQASGKERESVA |
| 270 | 6F3 | WYRQASGKERESVA |
| 271 | 1H2 | WVRQAPGKGLEWVS |
| 272 | 3F2 | WYRQAPGKEREMVA |
| 273 | 12C2 | WFRQAPGKQREWVA |
| 274 | 2D1 | WYRQAQGKQREPVA |
| 275 | 6H2 | WFRQAPGKERQFVA |
| 276 | 5D2 | WHRQAPGKQREPVA |
| 277 | 7C4 | WYRRAPGQVREMVA |
| 278 | 5F2 | WYRQAPGTERDLVA |
| 279 | 2C2 | WYRQAPGAQRELLA |
| 280 | 5G2 | WYRQAPGKQRELVA |
| 281 | 9H2 | WYRQAPGKERELVA |
| 282 | 5D4 | WYRQAPGKQRELVA |
| 283 | 2A4 | WYRQAPGNQRELVA |
| 284 | 7F1 | WYRQAPGKQREPVA |
| 285 | 5C2 | WFRQAPGEERELVA |
| 286 | 2F4 | WYRQAPGNQREPVA |
| 287 | 2A2 | WYRQAPGTERDLVA |
| 288 | 11F3 | WHRQAPGNERELVA |
| 289 | 10B3 | WYRQAPGKERELVA |
| 290 | MH1 | WYRQAPGKQRELVA |
| 291 | MH2 | WVRQAPGKGLEWVS |
| 292 | MH3 | WYRQAPGKERELVA |
| 293 | MH4 | WYRQAPGKERELVA |
| 294 | MH5 | WVRQAPGKGLEWVS |
| 295 | MH6 | WYRQAPGTERDLVA |
| 296 | MH7 | WYRQAPGKERELVA |
| 297 | MH8 | WVRQAPGKGLEWVS |
| 298 | MH9 | WHRQAPGNERELVA |
| 299 | MH10 | WHRQAPGKERELVA |
| 300 | MH11 | WVRQAPGKGLEWVS |

Framework region 3 (f3) of exemplary MSLN
trispecific binding proteins of this disclosure

| | Exemplary | |
|---|---|---|
| Sequence | trispecific | |
| ID No. | protein/TriTAC | Framework 3 |
| 301 | 9B1 | ISWDIAENTVYLQMNSLNSEDT |
| | | TVYYCNS |
| 302 | 9F3 | ISGDNVRNMVYLQMNSLKPEDT |
| | | AIYYCSA |
| 303 | 7H2 | ISGENGKNTVYLQMNSLKLEDT |
| | | AVYYCLG |
| 304 | 3B4 | ISRDNANNAIYLEMNSLKPEDT |
| | | AVYVCNA |
| 305 | 4A2 | ISRDNAENTVSLQMNTLKPEDT |
| | | AVYFCNA |
| 306 | 12D1 | ISKDSTRNTVYLQMNMLRPEDT |
| | | AVYYCNA |
| 307 | 3G1 | ISRDNAKNTVYLQMNSLKPEDT |
| | | AVYYCNA |
| 308 | 2A1 | ISRDIDKKTVYLQMDNLKPEDT |
| | | GVYYCNS |
| 309 | 6F3 | ISRDIYKKTVYLQMDNLKPEDT |
| | | GVYYCNS |
| 310 | 1H2 | ISRDNAKTTLYLQMNSLRPEDT |
| | | AVYYCTI |
| 311 | 3F2 | ISKENAKNTVYLQMNSLKPEDT |
| | | AVYYCHI |
| 312 | 12C2 | ISRDNAKSTVYLQMNSLKPEDT |
| | | AIYYCKA |
| 313 | 2D1 | ISRDNAKNTVYLQMNSLKPEDT |
| | | AIYTCHV |
| 314 | 6H2 | ISRDNAANTFYLQMNNLRPDDT |
| | | AVYYCNV |
| 315 | 5D2 | VSRDIVKNTMYLQMNSLKPEDT |
| | | AVYYCSY |
| 316 | 7C4 | ISRDNTQNLVYLQMNNLQPHDT |
| | | AIYYCGA |
| 317 | 5F2 | ISRDNAKNTLYLQMNNLKPEDT |
| | | AVYYCNA |
| 318 | 2C2 | ISRDNAKNTVYLQMNRLTPEDT |
| | | DVYYCRF |
| 319 | 5G2 | ISRDNAKNTVYLQMESLVAEDT |
| | | AVYYCNA |
| 320 | 9H2 | VSRDSAKNIVYLQMNSLTPEDT |
| | | AVYYCNT |
| 321 | 5D4 | ISRDNAKNTVYLQMNSLKPEDT |
| | | AVYYCNA |
| 322 | 2A4 | ISRDNAKNTVYLQMNSLKPEDT |
| | | AVYYCNT |
| 323 | 7F1 | ISRDNAKNTVYLQMNSLKPEDT |
| | | AIYTCHV |
| 324 | 5C2 | VSRDSAKNIVYLQMNRLKPEDT |
| | | AVYYCNT |
| 325 | 2F4 | ISRDNARNTVYLQMDSLKPEDT |
| | | AIYTCHV |
| 326 | 2A2 | ISRDNAKNTLYLQMNNLKPEDT |
| | | AVYYCNA |
| 327 | 11F3 | ISRDNAKNTAYLQMNSLKPEDT |
| | | AVYYCSV |
| 328 | 10B3 | VSRDSAKNIVYLQMNSLKPEDT |
| | | AVYYCNT |
| 329 | MH1 | ISRDNSKNTLYLQMNSLRAEDT |
| | | AVYYCAV |
| 330 | MH2 | ISRDNSKNTLYLQMNSLRAEDT |
| | | AVYYCAV |
| 331 | MH3 | ISRDNSKNTLYLQMNSLRAEDT |
| | | AVYYCNT |
| 332 | MH4 | ISRDNSKNTLYLQMNSLRAEDT |
| | | AVYYCNT |
| 333 | MH5 | ISRDNSKNTLYLQMNSLRAEDT |
| | | AVYYCNT |
| 334 | MH6 | ISRDNSKNTLYLQMNSLRAEDT |
| | | AVYYCNA |
| 335 | MH7 | ISRDNSKNTLYLQMNSLRAEDT |
| | | AVYYCNA |
| 336 | MH8 | ISRDNSKNTLYLQMNSLRAEDT |
| | | AVYYCNA |
| 337 | MH9 | ISRDNSKNTLYLQMNSLRAEDT |
| | | AVYYCSV |
| 338 | MH10 | ISRDNSKNTLYLQMNSLRAEDT |
| | | AVYYCSV |
| 339 | MH11 | ISRDNSKNTLYLQMNSLRAEDT |
| | | AVYYCSV |

Framework region 4 (f4) of exemplary MSLN
trispecific binding proteins of this disclosure

| | Exemplary | |
|---|---|---|
| Sequence | trispecific | |
| ID No. | protein/TriTAC | Framework 4 |
| 340 | 9B1 | WGQGTQVTVSS |
| 341 | 9F3 | WGKGTLVTVSS |
| 342 | 7H2 | WGQGTQVTVSS |
| 343 | 3B4 | FGQGTQVTVSS |
| 344 | 4A2 | WGQGTQVTVSS |
| 345 | 12D1 | WGQGTQVTVSS |
| 346 | 3G1 | WGQGTQVTVSS |
| 347 | 2A1 | WGQGTQVTVSS |
| 348 | 6F3 | WGQGTQVTVSS |
| 349 | 1H2 | QGTLVTVSS |
| 350 | 3F2 | WGQGTQVTVSS |
| 351 | 12C2 | WGQGTQVTVSS |
| 352 | 2D1 | WGQGTQVTVSS |
| 353 | 6H2 | WGQGTQVTVSS |
| 354 | 5D2 | WGQGTQVTVSS |
| 355 | 7C4 | WGQGTQVTVSS |
| 356 | 5F2 | WGQGTQVTVSS |
| 357 | 2C2 | WGQGTQVTVSS |
| 358 | 5G2 | WGQGTQVTVSS |
| 359 | 9H2 | WGQGTQVTVSS |
| 360 | 5D4 | WGQGTQVTVSS |
| 361 | 2A4 | WGQGTQVTVSS |
| 362 | 7F1 | WGQGTQVTVSS |
| 363 | 5C2 | WGQGTQVTVSS |
| 364 | 2F4 | WGQGTQVTVSS |
| 365 | 2A2 | WGQGTQVTVSS |
| 366 | 11F3 | WGQGTQVTVSS |
| 367 | 10B3 | WGQGTQVTVSS |
| 368 | MH1 | WGQGTLVTVSS |
| 369 | MH2 | WGQGTLVTVSS |
| 370 | MH3 | WGQGTLVTVSS |
| 371 | MH4 | WGQGTLVTVSS |
| 372 | MH5 | WGQGTLVTVSS |
| 373 | MH6 | WGQGTLVTVSSGG |
| 374 | MH7 | WGQGTLVTVSSGG |
| 375 | MH8 | WGQGTLVTVSSGG |
| 376 | MH9 | WGQGTLVTVS |
| 377 | MH10 | WGQGTLVTVSS |
| 378 | MH11 | WGQGTLVTVSS |

1-27. (canceled)

28. A method for the treatment or amelioration of proliferative disease, or a tumorous disease, comprising administration of a mesothelin (MSLN) binding trispecific protein comprising a sequence as set forth in any one of SEQ ID Nos.: 58-86, 98, 100, and 101.

29. The method of claim 28, comprising administering the MSLN binding trispecific protein at a dose of up to 10 mg/kg.

30. The method of claim 29, wherein the MSLN binding trispecific protein is administered once a week, twice per week, every other day, or every three weeks.

31. The method of claim 28, wherein the tumorous disease comprises a solid tumor disease.

32. The method of claim 31, wherein the solid tumor disease comprises mesothelioma, lung cancer, gastric cancer, ovarian cancer, or triple negative breast cancer.

33. The method of claim 31, wherein the solid tumor disease is metastatic.

34. The method of claim 28, wherein the MSLN binding trispecific protein selectively binds to tumor cells expressing mesothelin.

35. The method of claim 28, wherein the MSLN binding trispecific protein mediates T cell killing of the tumor cells expressing mesothelin.

36. The method of claim 28, wherein the proliferative disease is selected from the group consisting of leukemias, lymphomas, brain tumors, breast cancers, adrenal cancers, thyroid cancers, pancreatic cancers, pituitary cancers, eye cancers, vaginal cancers, vulvar cancers, cervical cancers, uterine cancers, ovarian cancers, stomach cancers, colon cancers, rectal cancer, liver cancers, gallbladder cancers, cholangiocarcinomas, lung cancers, testicular cancers, prostate cancers, oral cancers, salivary gland cancers, pharynx cancers, skin cancers, kidney cancers and bladder cancers.

37. The method of claim 28, wherein the proliferative disease is selected from the group consisting of myxo sarcoma, osteogenic sarcoma, endothelio sarcoma, lymphangioendothelio sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinoma.

38. The method of claim 28, wherein the MSLN binding trispecific protein is administered before, during, or after surgery.

39. The method of claim 28, furthering comprising administration of an agent in combination with the MSLN binding trispecific protein.

40. The method of claim 39, wherein the agent is selected from the group consisting of anti-diarrheal agents, anti-emetic agents, analgesics, opioids and non-steroidal anti-inflammatory agents.

41. The method of claim 39, wherein the agent is an anti-cancer agent.

42. The method of claim 41, wherein the anti-cancer agent is selected from the group consisting of acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacytidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzele sin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin II, interferon alpha-2a, interferon alpha-2b, interferon alpha-nl interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinzolidine sulfate, vinzolidine sulfate, vorozole, zeniplatin, zinostatin, zorubicin hydrochloride.

43. The method of claim 41, wherein the anti-cancer agent is selected from the group consisting of 20-epi-1, 25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorins, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, 9-dioxamycin, diphenyl spiromustine, docetaxel, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-I receptor inhibitor, interferon agonists, interferons, interleukins, iobenguane, iododoxorubicin, ipomeanol, 4-iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, HMG-CoA reductase inhibitor (such as but not limited to, Lovastatin, Pravastatin, Fluvastatin, Statin, Simvastatin, and Atorvastatin), loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, 06-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B 1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, Vitaxin®, vorozole, zanoterone, zeniplatin, zilascorb and zinostatin stimalamer.

44. The method of claim 41, wherein the anti-cancer agent comprises 5-fluorouracil and leucovorin.

45. The method of claim 41, wherein the anti-cancer agent comprises gemcitabine.

46. A method for the treatment or amelioration of proliferative disease, or a tumorous disease, comprising administration of a mesothelin (MSLN) binding trispecific protein comprising a sequence as set forth in SEQ ID No.: 98.

47. The method of claim 46, comprising administering the MSLN binding trispecific protein at a dose of up to 10 mg/kg.

48. The method of claim 47, wherein the MSLN binding trispecific protein is administered once a week, twice per week, every other day, or every three weeks.

49. The method of claim 46, wherein the tumorous disease comprises a solid tumor disease.

50. The method of claim 49, wherein the solid tumor disease comprises mesothelioma, lung cancer, gastric cancer, ovarian cancer, or triple negative breast cancer.

51. The method of claim 49, wherein the solid tumor disease is metastatic.

52. The method of claim 46, wherein the MSLN binding trispecific protein selectively binds to tumor cells expressing mesothelin.

53. The method of claim 46, wherein the MSLN binding trispecific protein mediates T cell killing of the tumor cells expressing mesothelin.

54. The method of claim 46, wherein the proliferative disease is selected from the group consisting of leukemias, lymphomas, brain tumors, breast cancers, adrenal cancers, thyroid cancers, pancreatic cancers, pituitary cancers, eye cancers, vaginal cancers, vulvar cancers, cervical cancers, uterine cancers, ovarian cancers, stomach cancers, colon cancers, rectal cancer, liver cancers, gallbladder cancers, cholangiocarcinomas, lung cancers, testicular cancers, prostate cancers, oral cancers, salivary gland cancers, pharynx cancers, skin cancers, kidney cancers and bladder cancers.

55. The method of claim 46, wherein the proliferative disease is selected from the group consisting of myxo sarcoma, osteogenic sarcoma, endothelio sarcoma, lymphangioendothelio sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinoma.

56. The method of claim 46, further comprising administration of an agent in combination with the MSLN binding trispecific protein.

57. The method of claim 46, wherein the agent is selected from the group consisting of anti-diarrheal agents, anti-emetic agents, analgesics, and non-steroidal anti-inflammatory agents.

58. The method of claim 46, wherein the agent is an anti-cancer agent.


SEQUENCE LISTING (379 sequences)

SEQ ID NO: 1 - protein, 110 aa, Artificial Sequence
  Description of Artificial Sequence Synthetic polypeptide
QVQLVESGGGLVQPGGSLRLSCAASGRTFSVRGMAWYRQAGNNRALVATMNPDGFPNYADAVKGRFTISWDIAENTVYLQMNSLNSEDTTVYYCNSGPYWGQGTQVTVSS

SEQ ID NO: 2 - protein, 123 aa, Artificial Sequence
  Description of Artificial Sequence Synthetic polypeptide
QVQLVESGGGLVQAGGSLRLSCAASGSIPSIEQMGWYRQAPGKQRELVAALTSGGRANYADSVKGRFTISGDNVRNMVYLQMNSLKPEDTAIYYCSAGRFKGDYAQRSGMDYWGKGTLVTVSS

SEQ ID NO: 3 - protein, 116 aa, Artificial Sequence
  Description of Artificial Sequence Synthetic polypeptide
QVQLVESGGGLVQAGGSLRLSCAFSGTTYTFDLMSWYRQAPGKQRTVVASISSDGRTSYADSVRGRFTISGENGKNTVYLQMNSLKLEDTAVYYCLGQRSGVRAFWGQGTQVTVSS

SEQ ID NO: 4 - protein, 123 aa, Artificial Sequence
  Description of Artificial Sequence Synthetic polypeptide
QVQLVESGGGLVQAGGSLRLSCVASGSTSNINNMRWYRQAPGKERELVAVITRGGYAIYLDAVKGRFTISRDNANNAIYLEMNSLKPEDTAVYVCNADRVEGTSGGPQLRDYFGQGTQVTVSS

SEQ ID NO: 5 - protein, 116 aa, Artificial Sequence
  Description of Artificial Sequence Synthetic polypeptide
QVQLVESGGGLVQAGGSLRLSCAASGSTFGINAMGWYRQAPGKQRELVAVISRGGSTNYADSVKGRFTISRDNAENTVSLQMNTLKPEDTAVYFCNARTYTRHDYWGQGTQVTVSS

SEQ ID NO: 6 - protein, 122 aa, Artificial Sequence
  Description of Artificial Sequence Synthetic polypeptide
QVRLVESGGGLVQAGGSLRLSCAASISAFRLMSVRWYRQDPSKQREWVATIDQLGRTNYADSVKGRFAISKDSTRNTVYLQMNMLRPEDTAVYYCNAGGGPLGSRWLRGRHWGQGTQVTVSS

SEQ ID NO: 7 - protein, 119 aa, Artificial Sequence
  Description of Artificial Sequence Synthetic polypeptide
QVRLVESGGGLVQAGESLRLSCAASGRPFSINTMGWYRQAPGKQRELVASISSSGDFTYTDSVKGRFTISRDNAKNTVYLQMNSLKPEDTAVYYCNARRTYLPRRFGSWGQGTQVTVSS

SEQ ID NO: 8 - protein, 117 aa, Artificial Sequence
  Description of Artificial Sequence Synthetic polypeptide
QVQPVESGGGLVQPGGSLRLSCVVSGSDFTEDAMAWYRQASGKERESVAFVSKDGKRILYLDSVRGRFTISRDIDKKTVYLQMDNLKPEDTGVYYCNSAPGAARNYWGQGTQVTVSS

SEQ ID NO: 9 - protein, 117 aa, Artificial Sequence
  Description of Artificial Sequence Synthetic polypeptide
QVQPVESGGGLVQPGGSLRLSCVVSGSDFTEDAMAWYRQASGKERESVAFVSKDGKRILYLDSVRGRFTISRDIYKKTVYLQMDNLKPEDTGVYYCNSAPGAARNVWGQGTQVTVSS

SEQ ID NO: 10 - protein, 115 aa, Artificial Sequence [sequence omitted]

SEQ ID NO: 11 - protein, 124 aa, Artificial Sequence
  Description of Artificial Sequence Synthetic polypeptide
QVQIVESGGGLVQAGGSLRLSCVASGLTYSIVAVGWYRQAPGKEREMVADISPVGNTNYADSVKGRFTISKENAKNTVYLQMNSLKPEDTAVYYCHIVRGWLDERPGPGPIVYWGQGTQVTVSS

SEQ ID NO: 12 - protein, 115 aa, Artificial Sequence
  Description of Artificial Sequence Synthetic polypeptide
QVQLVESGGGLVQTGGSLRLSCAASGLTFGVYGMEWFRQAPGKQREWVASHTSTGYVYYRDSVKGRFTISRDNAKSTVYLQMNSLKPEDTAIYYCKANRGSYEYWGQGTQVTVSS

SEQ ID NO: 13 - protein, 114 aa, Artificial Sequence
  Description of Artificial Sequence Synthetic polypeptide
QVQLVESGGGLVQAGGSLRLSCAASTTSSINSMSWYRQAQGKQREPVAVITDRGSTSYADSVKGRFTISRDNAKNTVYLQMNSLKPEDTAIYTCHVIADWRGYWGQGTQVTVSS

SEQ ID NO: 14 - protein, 120 aa, Artificial Sequence
  Description of Artificial Sequence Synthetic polypeptide
QVQLVESGGGLVQAGGSLRLSCAASGRTLSRYAMGWFRQAPGKERQFVAAISRSGGTTRYSDSVKGRFTISRDNAANTFYLQMNNLRPDDTAVYYCNVRRRGWGRTLEYWGQGTQVTVSS

SEQ ID NO: 15 - protein, 117 aa, Artificial Sequence
  Description of Artificial Sequence Synthetic polypeptide
QVQLGESGGGLVQAGGSLRLSCAASGSIFSPNAMIWHRQAPGKQREPVASINSSGSTNYGDSVKGRFTVSRDIVKNTMYLQMNSLKPEDTAVYYCSYSDFRRGTQYWGQGTQVTVSS

SEQ ID NO: 16 - protein, 119 aa, Artificial Sequence
  Description of Artificial Sequence Synthetic polypeptide
QVQLVESGGGLVPSGGSLRLSCAASGATSAITNLGWYRRAPGQVREMVARISVREDKEDYEDSVKGRFTISRDNTQNLVYLQMNNLQPHDTAIYYCGAQRWGRGPGTTWGQGTQVTVSS

SEQ ID NO: 17 - protein, 116 aa, Artificial Sequence
  Description of Artificial Sequence Synthetic polypeptide
QVQLVESGGGLVQAGGSLRLSCAASGSTFRIRVMRWYRQAPGTERDLVAVISGSSTYYADSVKGRFTISRDNAKNTLYLQMNNLKPEDTAVYYCNADDSGIARDYWGQGTQVTVSS

SEQ ID NO: 18 - protein, 118 aa, Artificial Sequence
  Description of Artificial Sequence Synthetic polypeptide
QVQLVESGGGLVQAGESRRLSCAVSGDTSKFKAVGWYRQAPGAQRELLAWINNSGVGNTAESVKGRFTISRDNAKNTVYLQMNRLTPEDTDVYYCRFYRRFGINKNYWGQGTQVTVSS

SEQ ID NO: 19 - protein, 122 aa, Artificial Sequence
  Description of Artificial Sequence Synthetic polypeptide
QVQLVESGGGLVQAGGSLRLSCAASGSTFGNKPMGWYRQAPGKQRELVAVISSDGGSTRYAALVKGRFTISRDNAKNTVYLQMESLVAEDTAVYYCNALRTYYLNDPVVFSWGQGTQVTVSS

SEQ ID NO: 20 - protein, 119 aa, Artificial Sequence
  Description of Artificial Sequence Synthetic polypeptide
QVQLVESGGGLVQAGGSLRLSCAASGSTSSINTMYWYRQAPGKERELVAFISSGGSTNVRDSVKGRFSVSRDSAKNIVYLQMNSLTPEDTAVYYCNTYIPLRGTLHDYWGQGTQVTVSS

SEQ ID NO: 21 - protein, 115 aa, Artificial Sequence
  Description of Artificial Sequence Synthetic polypeptide
QVQLVESGGGLVQAGGSLRLSCVASGRTDRITTMGWYRQAPGKQRELVATISNRGTSNYANSVKGRFTISRDNAKNTVYLQMNSLKPEDTAVYYCNARKWGRNYWGQGTQVTVSS

SEQ ID NO: 22 - protein, 119 aa, Artificial Sequence
  Description of Artificial Sequence Synthetic polypeptide
QVQLVESGGGLVQARGSLRLSCTASGRTIGINDMAWYRQAPGNQRELVATITKGGTTDYADSVDGRFTISRDNAKNTVYLQMNSLKPEDTAVYYCNTKRREWAKDFEYWGQGTQVTVSS

SEQ ID NO: 23 - protein, 114 aa, Artificial Sequence
  Description of Artificial Sequence Synthetic polypeptide
QVQLVESGGGLVQAGGSLRLSCAASAIGSINSMSWYRQAPGKQREPVAVITDRGSTSYADSVKGRFTISRDNAKNTVYLQMNSLKPEDTAIYTCHVIADWRGYWGQGTQVTVSS

SEQ ID NO: 24 - protein, 119 aa, Artificial Sequence
  Description of Artificial Sequence Synthetic polypeptide
QVQLVESGGGLVQAGGSLRLSCAASGSTSSINTMYWFRQAPGEERELVATINRGGSTNVRDSVKGRFSVSRDSAKNIVYLQMNRLKPEDTAVYYCNTYIPYGGTLHDFWGQGTQVTVSS

SEQ ID NO: 25 - protein, 114 aa, Artificial Sequence
  Description of Artificial Sequence Synthetic polypeptide
QVQLVESGGGLVQAGGSLRLSCTTSTTFSINSMSWYRQAPGNQREPVAVITNRGTTSYADSVKGRFTISRDNARNTVYLQMDSLKPEDTAIYTCHVIADWRGYWGQGTQVTVSS

SEQ ID NO: 26 - protein, 116 aa, Artificial Sequence
  Description of Artificial Sequence Synthetic polypeptide
QVQLVESGGGLVQAGGSLTLSCAASGSTFSIRAMRWYRQAPGTERDLVAVIYGSSTYYADAVKGRFTISRDNAKNTLYLQMNNLKPEDTAVYYCNADTIGTARDYWGQGTQVTVSS

SEQ ID NO: 27 - protein, 117 aa, Artificial Sequence
  Description of Artificial Sequence Synthetic polypeptide
QVQLVESGGGLVQAGGSLRLSCVASGRTSTIDTMYWHRQAPGNERELVAYVTSRGTSNVADSVKGRFTISRDNAKNTAYLQMNSLKPEDTAVYYCSVRTTSYPVDFWGQGTQVTVSS

SEQ ID NO: 28 - protein, 119 aa, Artificial Sequence
  Description of Artificial Sequence Synthetic polypeptide
QVQLVESGGGLVQAGGSLRLSCAASGSTSSINTMYWYRQAPGKERELVAFISSGGSTNVRDSVKGRFSVSRDSAKNIVYLQMNSLKPEDTAVYYCNTYIPYGGTLHDFWGQGTQVTVSS

SEQ ID NO: 29 - protein, 111 aa, Artificial Sequence
  Description of Artificial Sequence Synthetic polypeptide
QVQLVESGGGLVQPGGSLRLSCAASGGDWSANFMYWYRQAPGKQRELVARISGRGVVDYVESVKGRFTISRDNAKNTVYLQMNSLKPEDTAVYYCAVASYWGQGTQVTVSS

SEQ ID NO: 30 - protein, 111 aa, Artificial Sequence
  Description of Artificial Sequence Synthetic polypeptide
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 31 - protein, 111 aa, Artificial Sequence
  Description of Artificial Sequence Synthetic polypeptide
EVQLVESGGGLVQPGGSLRLSCAASGGDWSANFMYWVRQAPGKGLEWVSRISGRGVVDYVESVKGRFTISRDNSKNTLYLQMNSLRAEDTAVYYCAVASYWGQGTLVTVSS

SEQ ID NO: 32 - protein, 119 aa, Artificial Sequence
  Description of Artificial Sequence Synthetic polypeptide
EVQLVESGGGLVQAGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 33 - protein, 119 aa, Artificial Sequence
  Description of Artificial Sequence Synthetic polypeptide
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 34 - protein, 119 aa, Artificial Sequence
  Description of Artificial Sequence Synthetic polypeptide
EVQLVESGGGLVQPGGSLRLSCAASGSTSSINTMYWVRQAPGKGLEWVSFISSGGSTNVRDSVKGRFTISRDNSKNTLYLQMNSLRAEDTAVYYCNTYIPYGGTLHDFWGQGTLVTVSS

SEQ ID NO: 35 - protein, 118 aa, Artificial Sequence
  Description of Artificial Sequence Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSSGG

SEQ ID NO: 36 - protein, 118 aa, Artificial Sequence
  Description of Artificial Sequence Synthetic polypeptide
QVQLVESGGGVVQPGGSLRLSCAASGSTFSIRAMRWYRQAPGKERELVAVIYGSSTYYADAVKGRFTISRDNSKNTLYLQMNSLRAEDTAVYYCNADTIGTARDYWGQGTLVTVSSGG

SEQ ID NO: 37 - protein, 118 aa, Artificial Sequence
  Description of Artificial Sequence Synthetic polypeptide
QVQLVESGGGVVQPGGSLRLSCAASGSTFSIRAMRWVRQAPGKGLEWVSVIYGSSTYYADAVKGRFTISRDNSKNTLYLQMNSLRAEDTAVYYCNADTIGTARDYWGQGTLVTVSSGG

SEQ ID NO: 38 - protein, 116 aa, Artificial Sequence
  Description of Artificial Sequence Synthetic polypeptide
EVQLVESGGGLVQAGGSLRLSCVASGRTSTIDTMYWHRQAPGNERELVAYVTSRGTSNVADSVKGRFTISRDNSKNTLYLQMNSLRAEDTAVYYCSVRTTSYPVDFWGQGTLVTVS

SEQ ID NO: 39 - protein, 117 aa, Artificial Sequence
  Description of Artificial Sequence Synthetic polypeptide
EVQLVESGGGLVQPGGSLRLSCAASGRTSTIDTMYWHRQAPGKERELVAYVTSRGTSNVADSVKGRFTISRDNSKNTLYLQMNSLRAEDTAVYYCSVRTTSYPVDFWGQGTLVTVSS

SEQ ID NO: 40 - protein, 117 aa, Artificial Sequence
  Description of Artificial Sequence Synthetic polypeptide
EVQLVESGGGLVQPGGSLRLSCAASGRTSTIDTMYWVRQAPGKGLEWVSYVTSRGTSNVADSVKGRFTISRDNSKNTLYLQMNSLRAEDTAVYYCSVRTTSYPVDFWGQGTLVTVSS

SEQ ID NO: 41 - protein, 7 aa, Artificial Sequence
  Description of Artificial Sequence Synthetic peptide
ESGGGLV

SEQ ID NO: 42 - protein, 3 aa, Artificial Sequence
  Description of Artificial Sequence Synthetic peptide
LSC

SEQ ID NO: 43 - protein, 3 aa, Artificial Sequence
  Description of Artificial Sequence Synthetic peptide
GRF

SEQ ID NO: 44 - protein, 5 aa, Artificial Sequence
  Description of Artificial Sequence Synthetic peptide
VTVSS

SEQ ID NO: 45 - protein, 8 aa, Artificial Sequence
  Description of Artificial Sequence Synthetic peptide
QLVESGGG

SEQ ID NO: 46 - protein, 12 aa, Artificial Sequence
  Description of Artificial Sequence Synthetic peptide
GGSLRLSCAASG

SEQ ID NO: 47 - protein, 3 aa, Artificial Sequence
  Description of Artificial Sequence Synthetic peptide
ASG

SEQ ID NO: 48 - protein, 5 aa, Artificial Sequence
  Description of Artificial Sequence Synthetic peptide
RQAPG

SEQ ID NO: 49 - protein, 33 aa, Artificial Sequence
  Description of Artificial Sequence Synthetic polypeptide
VKGRFTISRDNSKNTLYLQMNSLRAEDTAVYYC

SEQ ID NO: 50 - protein, 11 aa, Artificial Sequence
  Description of Artificial Sequence Synthetic peptide
WGQGTLVTVSS

SEQ ID NO: 51 - protein, 10 aa, Artificial Sequence
  Description of Artificial Sequence Synthetic peptide
GRTFSVRGMA

SEQ ID NO: 52 - protein, 10 aa, Artificial Sequence
  Description of Artificial Sequence Synthetic peptide
INSSGSTNYG

SEQ ID NO: 53 - protein, 10 aa, Artificial Sequence
  Description of Artificial Sequence Synthetic peptide
NAGGGPLGSR

SEQ ID NO: 54 - protein, 10 aa, Artificial Sequence
  Description of Artificial Sequence Synthetic peptide
GGDWSANFMY

SEQ ID NO: 55 - protein, 10 aa, Artificial Sequence
  Description of Artificial Sequence Synthetic peptide
ISSGGSTNVR

SEQ ID NO: 56 - protein, 10 aa, Artificial Sequence
  Description of Artificial Sequence Synthetic peptide
NADTIGTARD

SEQ ID NO: 57 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 58 - protein, 498 aa, Artificial Sequence
  Description of Artificial Sequence Synthetic polypeptide
QVQLVESGGGLVQPGGSLRLSCAASGRTFSVRGMAWYRQAGNNRALVATMNPDGFPNYADAVKGRFTISWDIAENTVYLQMNSLNSEDTTVYYCNSGPYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 59 - protein, 511 aa, Artificial Sequence
  Description of Artificial Sequence Synthetic polypeptide
QVQLVESGGGLVQAGGSLRLSCAASGSIPSIEQMGWYRQAPGKQRELVAALTSGGRANYADSVKGRFTISGDNVRNMVYLQMNSLKPEDTAIYYCSAGRFKGDYAQRSGMDYWGKGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 60 - protein, 504 aa, Artificial Sequence
  Description of Artificial Sequence Synthetic polypeptide
QVQLVESGGGLVQAGGSLRLSCAFSGTTYTFDLMSWYRQAPGKQRTVVASISSDGRTSYADSVRGRFTISGENGKNTVYLQMNSLKLEDTAVYYCLGQRSGVRAFWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 61 - protein, 511 aa, Artificial Sequence
  Description of Artificial Sequence Synthetic polypeptide
QVQLVESGGGLVQAGGSLRLSCVASGSTSNINNMRWYRQAPGKERELVAVITRGGYAIYLDAVKGRFTISRDNANNAIYLEMNSLKPEDTAVYVCNADRVEGTSGGPQLRDYFGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 62 - protein, 504 aa, Artificial Sequence
  Description of Artificial Sequence Synthetic polypeptide
QVQLVESGGGLVQAGGSLRLSCAASGSTFGINAMGWYRQAPGKQRELVAVISRGGSTNYADSVKGRFTISRDNAENTVSLQMNTLKPEDTAVYFCNARTYTRHDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 63 - protein, 510 aa, Artificial Sequence
  Description of Artificial Sequence Synthetic polypeptide
QVRLVESGGGLVQAGGSLRLSCAASISAFRLMSVRWYRQDPSKQREWVATIDQLGRTNYADSVKGRFAISKDSTRNTVYLQMNMLRPEDTAVYYCNAGGGPLGSRWLRGRHWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 64 - protein, 507 aa, Artificial Sequence
  Description of Artificial Sequence Synthetic polypeptide
QVRLVESGGGLVQAGESLRLSCAASGRPFSINTMGWYRQAPGKQRELVASISSSGDFTYTDSVKGRFTISRDNAKNTVYLQMNSLKPEDTAVYYCNARRTYLPRRFGSWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 65 - protein, 505 aa, Artificial Sequence
  Description of Artificial Sequence Synthetic polypeptide
QVQPVESGGGLVQPGGSLRLSCVVSGSDFTEDAMAWYRQASGKERESVAFVSKDGKRILYLDSVRGRFTISRDIDKKTVYLQMDNLKPEDTGVYYCNSAPGAARNYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 66 - protein, 505 aa, Artificial Sequence
  Description of Artificial Sequence Synthetic polypeptide
QVQPVESGGGLVQPGGSLRLSCVVSGSDFTEDAMAWYRQASGKERESVAFVSKDGKRILYLDSVRGRFTISRDIYKKTVYLQMDNLKPEDTGVYYCNSAPGAARNVWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 67 - protein, 503 aa, Artificial Sequence
  Description of Artificial Sequence Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 68 - protein, 512 aa, Artificial Sequence
  Description of Artificial Sequence Synthetic polypeptide
QVQIVESGGGLVQAGGSLRLSCVASGLTYSIVAVGWYRQAPGKEREMVADISPVGNTNYADSVKGRFTISKENAKNTVYLQMNSLKPEDTAVYYCHIVRGWLDERPGPGPIVYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 69 - protein, 503 aa, Artificial Sequence
  Description of Artificial Sequence Synthetic polypeptide
QVQLVESGGGLVQTGGSLRLSCAASGLTFGVYGMEWFRQAPGKQREWVASHTSTGYVYYRDSVKGRFTISRDNAKSTVYLQMNSLKPEDTAIYYCKANRGSYEYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 70 - protein, 502 aa, Artificial Sequence
  Description of Artificial Sequence Synthetic polypeptide
QVQLVESGGGLVQAGGSLRLSCAASTTSSINSMSWYRQAQGKQREPVAVITDRGSTSYADSVKGRFTISRDNAKNTVYLQMNSLKPEDTAIYTCHVIADWRGYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 71 - protein, 508 aa, Artificial Sequence
  Description of Artificial Sequence Synthetic polypeptide
QVQLVESGGGLVQAGGSLRLSCAASGRTLSRYAMGWFRQAPGKERQFVAAISRSGGTTRYSDSVKGRFTISRDNAANTFYLQMNNLRPDDTAVYYCNVRRRGWGRTLEYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 72 - protein, 505 aa, Artificial Sequence
  Description of Artificial Sequence Synthetic polypeptide
QVQLGESGGGLVQAGGSLRLSCAASGSIFSPNAMIWHRQAPGKQREPVASINSSGSTNYGDSVKGRFTVSRDIVKNTMYLQMNSLKPEDTAVYYCSYSDFRRGTQYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 73 - protein, 507 aa, Artificial Sequence
  Description of Artificial Sequence Synthetic polypeptide
QVQLVESGGGLVPSGGSLRLSCAASGATSAITNLGWYRRAPGQVREMVARISVREDKEDYEDSVKGRFTISRDNTQNLVYLQMNNLQPHDTAIYYCGAQRWGRGPGTTWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 74 - protein, 504 aa, Artificial Sequence
  Description of Artificial Sequence Synthetic polypeptide
QVQLVESGGGLVQAGGSLRLSCAASGSTFRIRVMRWYRQAPGTERDLVAVISGSSTYYADSVKGRFTISRDNAKNTLYLQMNNLKPEDTAVYYCNADDSGIARDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 75 - protein, 506 aa, Artificial Sequence
  Description of Artificial Sequence Synthetic polypeptide
QVQLVESGGGLVQAGESRRLSCAVSGDTSKFKAVGWYRQAPGAQRELLAWINNSGVGNTAESVKGRFTISRDNAKNTVYLQMNRLTPEDTDVYYCRFYRRFGINKNYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 76 - protein, 510 aa, Artificial Sequence
  Description of Artificial Sequence Synthetic polypeptide
QVQLVESGGGLVQAGGSLRLSCAASGSTFGNKPMGWYRQAPGKQRELVAVISSDGGSTRYAALVKGRFTISRDNAKNTVYLQMESLVAEDTAVYYCNALRTYYLNDPVVFSWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 77 - protein, 507 aa, Artificial Sequence
  Description of Artificial Sequence Synthetic polypeptide
QVQLVESGGGLVQAGGSLRLSCAASGSTSSINTMYWYRQAPGKERELVAFISSGGSTNVRDSVKGRFSVSRDSAKNIVYLQMNSLTPEDTAVYYCNTYIPLRGTLHDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 78 - protein, 503 aa, Artificial Sequence
  Description of Artificial Sequence Synthetic polypeptide
QVQLVESGGGLVQAGGSLRLSCVASGRTDRITTMGWYRQAPGKQRELVATISNRGTSNYANSVKGRFTISRDNAKNTVYLQMNSLKPEDTAVYYCNARKWGRNYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 79 - protein, 507 aa, Artificial Sequence
  Description of Artificial Sequence Synthetic polypeptide
QVQLVESGGGLVQARGSLRLSCTASGRTIGINDMAWYRQAPGNQRELVATITKGGTTDYADSVDGRFTISRDNAKNTVYLQMNSLKPEDTAVYYCNTKRREWAKDFEYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 80 - protein, 502 aa, Artificial Sequence
  Description of Artificial Sequence Synthetic polypeptide
QVQLVESGGGLVQAGGSLRLSCAASAIGSINSMSWYRQAPGKQREPVAVITDRGSTSYADSVKGRFTISRDNAKNTVYLQMNSLKPEDTAIYTCHVIADWRGYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 81 - protein, 507 aa, Artificial Sequence
  Description of Artificial Sequence Synthetic polypeptide
QVQLVESGGGLVQAGGSLRLSCAASGSTSSINTMYWFRQAPGEERELVATINRGGSTNVRDSVKGRFSVSRDSAKNIVYLQMNRLKPEDTAVYYCNTYIPYGGTLHDFWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 82 - protein, 502 aa, Artificial Sequence
  Description of Artificial Sequence Synthetic polypeptide
QVQLVESGGGLVQAGGSLRLSCTTSTTFSINSMSWYRQAPGNQREPVAVITNRGTTSYADSVKGRFTISRDNARNTVYLQMDSLKPEDTAIYTCHVIADWRGYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 83 - protein, 504 aa, Artificial Sequence
  Description of Artificial Sequence Synthetic polypeptide
QVQLVESGGGLVQAGGSLTLSCAASGSTFSIRAMRWYRQAPGTERDLVAVIYGSSTYYADAVKGRFTISRDNAKNTLYLQMNNLKPEDTAVYYCNADTIGTARDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 84 - protein, 505 aa, Artificial Sequence
  Description of Artificial Sequence Synthetic polypeptide
QVQLVESGGGLVQAGGSLRLSCVASGRTSTIDTMYWHRQAPGNERELVAYVTSRGTSNVADSVKGRFTISRDNAKNTAYLQMNSLKPEDTAVYYCSVRTTSYPVDFWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 85 - protein, 507 aa, Artificial Sequence
  Description of Artificial Sequence Synthetic polypeptide
QVQLVESGGGLVQAGGSLRLSCAASGSTSSINTMYWYRQAPGKERELVAFISSGGSTNVRDSVKGRFSVSRDSAKNIVYLQMNSLKPEDTAVYYCNTYIPYGGTLHDFWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 86 - protein, 499 aa, Artificial Sequence
  Description of Artificial Sequence Synthetic polypeptide
QVQLVESGGGLVQPGGSLRLSCAASGGDWSANFMYWYRQAPGKQRELVARISGRGVVDYVESVKGRFTISRDNAKNTVYLQMNSLKPEDTAVYYCAVASYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 87 - protein, 20 aa, Artificial Sequence
  Description of Artificial Sequence Synthetic peptide
  MISC_FEATURE (1)..(20): This sequence may encompass 1-10 “Gly Ser” repeating units
GSGSGSGSGSGSGSGSGSGS

SEQ ID NO: 88 - protein, 30 aa, Artificial Sequence
  Description of Artificial Sequence Synthetic polypeptide
  MISC_FEATURE (1)..(30): This sequence may encompass 1-10 “Gly Gly Ser” repeating units
GGSGGSGGSGGSGGSGGSGGSGGSGGSGGS

SEQ ID NO: 89 - protein, 40 aa, Artificial Sequence
  Description of Artificial Sequence Synthetic polypeptide
  MISC_FEATURE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 90 - protein, 40 aa, Artificial Sequence
  Description of Artificial Sequence Synthetic polypeptide
  MISC_FEATURE (1)..(40): This sequence may encompass 1-10 “Gly Gly Ser Gly” repeating units
GGSGGGSGGGSGGGSGGGSGGGSGGGSGGGSGGGSGGGSG

SEQ ID NO: 91 - protein, 50 aa, Artificial Sequence
  Description of Artificial Sequence Synthetic polypeptide
  MISC_FEATURE (1)..(50): This sequence may encompass 1-10 “Gly Gly Ser Gly Gly” repeating units
GGSGGGGSGGGGSGGGGSGGGGSGGGGSGGGGSGGGGSGGGGSGGGGSGG

SEQ ID NO: 92 - protein, 50 aa, Artificial Sequence
  Description of Artificial Sequence Synthetic polypeptide
  MISC_FEATURE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

SEQ ID NO: 93 - protein, 50 aa, Artificial Sequence
  Description of Artificial Sequence Synthetic polypeptide
  MISC_FEATURE (1)..(50): This sequence may encompass 1-10 “Gly Gly Gly Gly Gly” repeating units
GGGGGGGGGGGGGGGGGGGGGGGGGGGGGGGGGGGGGGGGGGGGGGGGGG

SEQ ID NO: 94 - protein, 30 aa, Artificial Sequence
  Description of Artificial Sequence Synthetic polypeptide
  MISC_FEATURE (1)..(30): This sequence may encompass 1-10 “Gly Gly Gly” repeating units
GGGGGGGGGGGGGGGGGGGGGGGGGGGGGG

SEQ ID NO: 95 - protein, 20 aa, Artificial Sequence
  Description of Artificial Sequence Synthetic peptide
GGGGSGGGGSGGGGSGGGGS

SEQ ID NO: 96 - protein, 15 aa, Artificial Sequence
  Description of Artificial Sequence Synthetic peptide
GGGGSGGGGSGGGGS

SEQ ID NO: 97 - protein, 5 aa, Unknown
  Description of Unknown sortase recognition domain
LPETG

SEQ ID NO: 98 - protein, 504 aa, Artificial Sequence
  Description of Artificial Sequence Synthetic polypeptide
QVQLVESGGGVVQAGGSLTLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 99 - protein, 503 aa, Artificial Sequence
  Description of Artificial Sequence Synthetic polypeptide
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 100 - protein, 504 aa, Artificial Sequence
  Description of Artificial Sequence Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGNTFNKYAMNWVRQAPGKGLEWVARIRSKYNNYATYYADSVKDRFTISRDDSKNTAYLQMNNLKTEDTAVYYCVRHGNFGDSYISYWAYWGQGTLVTVSSGGGGSGGGGSGGGGSQTVVTQEPSLTVSPGGTVTLTCGSSTGAVTHGNYPNWVQQKPGQAPRGLIGGTKVLAPGTPARFSGSLLGGKAALTLSGVQPEDEAEYYCVLWYSNRWVFGGGTKLTVLHHHHHH

SEQ ID NO: 101 - protein, 504 aa, Artificial Sequence
  Description of Artificial Sequence Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 102 - protein, 116 aa, Artificial Sequence
  Description of Artificial Sequence Synthetic polypeptide
QVQLVESGGGVVQAGGSLTLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 103 - protein, 116 aa, Artificial Sequence
  Description of Artificial Sequence Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 104 - protein, 116 aa, Artificial Sequence
  Description of Artificial Sequence Synthetic polypeptide
QVQLVESGGGVVQPGGSLRLSCAASGSTFSIRAMRWYRQAPGKERELVAVIYGSSTYYADAVKGRFTISRDNSKNTLYLQMNSLRAEDTAVYYCNADTIGTARDYWGQGTLVTVSS

SEQ ID NO: 105 - protein, 116 aa, Artificial Sequence
  Description of Artificial Sequence Synthetic polypeptide
QVQLVESGGGVVQPGGSLRLSCAASGSTFSIRAMRWVRQAPGKGLEWVSVIYGSSTYYADAVKGRFTISRDNSKNTLYLQMNSLRAEDTAVYYCNADTIGTARDYWGQGTLVTVSS

SEQ ID NO: 106 - protein, 10 aa, Artificial Sequence
  Description of Artificial Sequence Synthetic peptide
GRTFSVRGMA

SEQ ID NO: 107 - protein, 10 aa, Artificial Sequence
  Description of Artificial Sequence Synthetic peptide
GSIPSIEQMG

SEQ ID NO: 108 - protein, 10 aa, Artificial Sequence
  Description of Artificial Sequence Synthetic peptide
GTTYTFDLMS

SEQ ID NO: 109 - protein, 10 aa, Artificial Sequence
  Description of Artificial Sequence Synthetic peptide
GSTSNINNMR

SEQ ID NO: 110 - protein, 10 aa, Artificial Sequence
  Description of Artificial Sequence Synthetic peptide
GSTFGINAMG

SEQ ID NO: 111 - protein, 10 aa, Artificial Sequence
  Description of Artificial Sequence Synthetic peptide
ISAFRLMSVR

SEQ ID NO: 112 - protein, 10 aa, Artificial Sequence
  Description of Artificial Sequence Synthetic peptide
GRPFSINTMG

SEQ ID NO: 113 - protein, 10 aa, Artificial Sequence
  Description of Artificial Sequence Synthetic peptide
GSDFTEDAMA

SEQ ID NO: 114 - protein, 10 aa, Artificial Sequence
  Description of Artificial Sequence Synthetic peptide
GSDFTEDAMA

SEQ ID NO: 115 - protein, 10 aa, Artificial Sequence
  Description of Artificial Sequence Synthetic peptide
GFTFSSFGMS

SEQ ID NO: 116 - protein, 10 aa, Artificial Sequence
  Description of Artificial Sequence Synthetic peptide
GLTYSIVAVG

SEQ ID NO: 117 - protein, 10 aa, Artificial Sequence
  Description of Artificial Sequence Synthetic peptide
GLTFGVYGME

SEQ ID NO: 118 - protein, 9 aa, Artificial Sequence
  Description of Artificial Sequence Synthetic peptide
TTSSINSMS

SEQ ID NO: 119 - protein, 10 aa, Artificial Sequence
  Description of Artificial Sequence Synthetic peptide
GRTLSRYAMG

SEQ ID NO: 120 - protein, 10 aa, Artificial Sequence
  Description of Artificial Sequence Synthetic peptide
GSIFSPNAMI

SEQ ID NO: 121 - protein, 10 aa, Artificial Sequence
  Description of Artificial Sequence Synthetic peptide
GATSAITNLG

SEQ ID NO: 122 - protein, 10 aa, Artificial Sequence
  Description of Artificial Sequence Synthetic peptide
GSTFRIRVMR

SEQ ID NO: 123 - protein, 10 aa, Artificial Sequence
  Description of Artificial Sequence Synthetic peptide
GDTSKFKAVG

SEQ ID NO: 124 - protein, 10 aa, Artificial Sequence
  Description of Artificial Sequence Synthetic peptide
GSTFGNKPMG

SEQ ID NO: 125 - protein, 10 aa, Artificial Sequence
  Description of Artificial Sequence Synthetic peptide
GSTSSINTMY

SEQ ID NO: 126 - protein, 10 aa, Artificial Sequence
  Description of Artificial Sequence Synthetic peptide
GRTDRITTMG

SEQ ID NO: 127 - protein, 10 aa, Artificial Sequence
  Description of Artificial Sequence Synthetic peptide
GRTIGINDMA

SEQ ID NO: 128 - protein, 9 aa, Artificial Sequence
  Description of Artificial Sequence Synthetic peptide
AIGSINSMS

SEQ ID NO: 129 - protein, 10 aa, Artificial Sequence
  Description of Artificial Sequence Synthetic peptide
GSTSSINTMY

SEQ ID NO: 130 - protein, 9 aa, Artificial Sequence
  Description of Artificial Sequence Synthetic peptide
TTFSINSMS

SEQ ID NO: 131 - protein, 10 aa, Artificial Sequence
  Description of Artificial Sequence Synthetic peptide
GSTFSIRAMR

SEQ ID NO: 132 - protein, 10 aa, Artificial Sequence
  Description of Artificial Sequence Synthetic peptide
GRTSTIDTMY

SEQ ID NO: 133 - protein, 10 aa, Artificial Sequence
  Description of Artificial Sequence Synthetic peptide
GSTSSINTMY

SEQ ID NO: 134 - protein, 10 aa, Artificial Sequence
  Description of Artificial Sequence Synthetic peptide
GGDWSANFMY

SEQ ID NO: 135 - protein, 10 aa, Artificial Sequence
  Description of Artificial Sequence Synthetic peptide
GGDWSANFMY

SEQ ID NO: 136 - protein, 10 aa, Artificial Sequence
  Description of Artificial Sequence Synthetic peptide
GSTSSINTMY

SEQ ID NO: 137 - protein, 10 aa, Artificial Sequence
  Description of Artificial Sequence Synthetic peptide
GSTSSINTMY

SEQ ID NO: 138 - protein, 10 aa, Artificial Sequence
  Description of Artificial Sequence Synthetic peptide
GSTSSINTMY

SEQ ID NO: 139 - protein, 10 aa, Artificial Sequence
  Description of Artificial Sequence Synthetic peptide
GSTFSIRAMR

SEQ ID NO: 140 - protein, 10 aa, Artificial Sequence
  Description of Artificial Sequence Synthetic peptide
GSTFSIRAMR

SEQ ID NO: 141 - protein, 10 aa, Artificial Sequence
  Description of Artificial Sequence Synthetic peptide
GSTFSIRAMR

SEQ ID NO: 142 - protein, 10 aa, Artificial Sequence
  Description of Artificial Sequence Synthetic peptide
GRTSTIDTMY

SEQ ID NO: 143 - protein, 10 aa, Artificial Sequence
  Description of Artificial Sequence Synthetic peptide
GRTSTIDTMY

SEQ ID NO: 144 - protein, 10 aa, Artificial Sequence
  Description of Artificial Sequence Synthetic peptide
GRTSTIDTMY

SEQ ID NO: 145 - protein, 19 aa, Artificial Sequence
  Description of Artificial Sequence Synthetic peptide
TMNPDGFPNYADAVKGRFT

SEQ ID NO: 146 - protein, 19 aa, Artificial Sequence
  Description of Artificial Sequence Synthetic peptide
ALTSGGRANYADSVKGRFT

SEQ ID NO: 147 - protein, 19 aa, Artificial Sequence
  Description of Artificial Sequence Synthetic peptide
SISSDGRTSYADSVRGRFT

SEQ ID NO: 148 - protein, 19 aa, Artificial Sequence
  Description of Artificial Sequence Synthetic peptide
VITRGGYAIYLDAVKGRFT

SEQ ID NO: 149 - protein, 19 aa, Artificial Sequence
  Description of Artificial Sequence Synthetic peptide
VISRGGSTNYADSVKGRFT

SEQ ID NO: 150 - protein, 19 aa, Artificial Sequence
  Description of Artificial Sequence Synthetic peptide
TIDQLGRTNYADSVKGRFA

SEQ ID NO: 151 - protein, 19 aa, Artificial Sequence
  Description of Artificial Sequence Synthetic peptide
SISSSGDFTYTDSVKGRFT

SEQ ID NO: 152 - protein, 20 aa, Artificial Sequence
  Description of Artificial Sequence Synthetic peptide
FVSKDGKRILYLDSVRGRFT

SEQ ID NO: 153 - protein, 20 aa, Artificial Sequence
  Description of Artificial Sequence Synthetic peptide
FVSKDGKRILYLDSVRGRFT

SEQ ID NO: 154 - protein, 20 aa, Artificial Sequence
  Description of Artificial Sequence Synthetic peptide
SISGSGSDTLYADSVKGRFT

SEQ ID NO: 155 - protein, 19 aa, Artificial Sequence
  Description of Artificial Sequence Synthetic peptide
DISPVGNTNYADSVKGRFT

SEQ ID NO: 156 - protein, 19 aa, Artificial Sequence
  Description of Artificial Sequence Synthetic peptide
SHTSTGYVYYRDSVKGRFT

SEQ ID NO: 157 - protein, 19 aa, Artificial Sequence
  Description of Artificial Sequence Synthetic peptide
VITDRGSTSYADSVKGRFT

SEQ ID NO: 158 - protein, 20 aa, Artificial Sequence
  Description of Artificial Sequence Synthetic peptide
AISRSGGTTRYSDSVKGRFT

SEQ ID NO: 159 - protein, 19 aa, Artificial Sequence
  Description of Artificial Sequence Synthetic peptide
SINSSGSTNYGDSVKGRFT

SEQ ID NO: 160 - protein, 20 aa, Artificial Sequence
  Description of Artificial Sequence Synthetic peptide
RISVREDKEDYEDSVKGRFT

SEQ ID NO: 161 - protein, 18 aa, Artificial Sequence
  Description of Artificial Sequence Synthetic peptide
VISGSSTYYADSVKGRFT

SEQ ID NO: 162 - protein, 19 aa, Artificial Sequence
  Description of Artificial Sequence Synthetic peptide
WINNSGVGNTAESVKGRFT

SEQ ID NO: 163 - protein, 20 aa, Artificial Sequence
  Description of Artificial Sequence Synthetic peptide
VISSDGGSTRYAALVKGRFT

SEQ ID NO: 164 - protein, 19 aa, Artificial Sequence
  Description of Artificial Sequence Synthetic peptide
FISSGGSTNVRDSVKGRFS

SEQ ID NO: 165 - protein, 19 aa, Artificial Sequence
  Description of Artificial Sequence Synthetic peptide
TISNRGTSNYANSVKGRFT

SEQ ID NO: 166 - protein, 19 aa, Artificial Sequence
  Description of Artificial Sequence Synthetic peptide
TITKGGTTDYADSVDGRFT

SEQ ID NO: 167 - protein, 19 aa, Artificial Sequence
  Description of Artificial Sequence Synthetic peptide
VITDRGSTSYADSVKGRFT

SEQ ID NO: 168 - protein, 19 aa, Artificial Sequence
  Description of Artificial Sequence Synthetic peptide
TINRGGSTNVRDSVKGRFS

SEQ ID NO: 169 - protein, 19 aa, Artificial Sequence
  Description of Artificial Sequence Synthetic peptide
VITNRGTTSYADSVKGRFT

SEQ ID NO: 170 - protein, 18 aa, Artificial Sequence
  Description of Artificial Sequence Synthetic peptide
VIYGSSTYYADAVKGRFT

SEQ ID NO: 171 - protein, 19 aa, Artificial Sequence
  Description of Artificial Sequence Synthetic peptide
YVTSRGTSNVADSVKGRFT

SEQ ID NO: 172 - protein, 19 aa, Artificial Sequence
  Description of Artificial Sequence Synthetic peptide
FISSGGSTNVRDSVKGRFS

SEQ ID NO: 173 - protein, 19 aa, Artificial Sequence
  Description of Artificial Sequence Synthetic peptide
RISGRGVVDYVESVKGRFT

SEQ ID NO: 174 - protein, 19 aa, Artificial Sequence
  Description of Artificial Sequence Synthetic peptide
RISGRGVVDYVESVKGRFT

SEQ ID NO: 175 - protein, 19 aa, Artificial Sequence
  Description of Artificial Sequence Synthetic peptide
FISSGGSTNVRDSVKGRFT

SEQ ID NO: 176 - protein, 19 aa, Artificial Sequence
  Description of Artificial Sequence Synthetic peptide
FISSGGSTNVRDSVKGRFT

SEQ ID NO: 177 - protein, 19 aa, Artificial Sequence
  Description of Artificial Sequence Synthetic peptide
FISSGGSTNVRDSVKGRFT

SEQ ID NO: 178 - protein, 18 aa, Artificial Sequence
  Description of Artificial Sequence Synthetic peptide
VIYGSSTYYADAVKGRFT

SEQ ID NO: 179 - protein, 18 aa, Artificial Sequence
  Description of Artificial Sequence Synthetic peptide
VIYGSSTYYADAVKGRFT

SEQ ID NO: 180 - protein, 18 aa, Artificial Sequence
  Description of Artificial Sequence Synthetic peptide
VIYGSSTYYADAVKGRFT

SEQ ID NO: 181 - protein, 19 aa, Artificial Sequence
  Description of Artificial Sequence Synthetic peptide
YVTSRGTSNVADSVKGRFT

SEQ ID NO: 182 - protein, 19 aa, Artificial Sequence
  Description of Artificial Sequence Synthetic peptide
YVTSRGTSNVADSVKGRFT

SEQ ID NO: 183 - protein, 19 aa, Artificial Sequence
  Description of Artificial Sequence Synthetic peptide
YVTSRGTSNVADSVKGRFT

SEQ ID NO: 184 - protein, 3 aa, Artificial Sequence
  Description of Artificial Sequence Synthetic peptide
GPY

SEQ ID NO: 185 - protein, 15 aa, Artificial Sequence
  Description of Artificial Sequence Synthetic peptide
GRFKGDYAQRSGMDY

SEQ ID NO: 186 - protein, 8 aa, Artificial Sequence
  Description of Artificial Sequence Synthetic peptide
QRSGVRAF

SEQ ID NO: 187 - protein, 15 aa, Artificial Sequence
  Description of Artificial Sequence Synthetic peptide
DRVEGTSGGPQLRDY

SEQ ID NO: 188 - protein, 8 aa, Artificial Sequence
  Description of Artificial Sequence Synthetic peptide
RTYTRHDY

SEQ ID NO: 189 - protein, 14 aa, Artificial Sequence
  Description of Artificial Sequence Synthetic peptide
GGGPLGSRWLRGRH

SEQ ID NO: 190 - protein, 11 aa, Artificial Sequence
  Description of Artificial Sequence Synthetic peptide
RRTYLPRRFGS

SEQ ID NO: 191 - protein, 8 aa, Artificial Sequence
  Description of Artificial Sequence Synthetic peptide
APGAARNY

SEQ ID NO: 192 - protein, 8 aa, Artificial Sequence
  Description of Artificial Sequence Synthetic peptide
APGAARNV

SEQ ID NO: 193 - protein, 8 aa, Artificial Sequence
  Description of Artificial Sequence Synthetic peptide
GGSLSRSS

SEQ ID NO: 194 - protein, 16 aa, Artificial Sequence
  Description of Artificial Sequence Synthetic peptide
VRGWLDERPGPGPIVY

SEQ ID NO: 195 - protein, 7 aa, Artificial Sequence
  Description of Artificial Sequence Synthetic peptide
NRGSYEY

SEQ ID NO: 196 - protein, 7 aa, Artificial Sequence
  Description of Artificial Sequence Synthetic peptide
IADWRGY

SEQ ID NO: 197 - protein, 11 aa, Artificial Sequence
  Description of Artificial Sequence Synthetic peptide
RRRGWGRTLEY

SEQ ID NO: 198 - protein, 9 aa, Artificial Sequence
  Description of Artificial Sequence Synthetic peptide
SDFRRGTQY

SEQ ID NO: 199 - protein, 10 aa, Artificial Sequence
  Description of Artificial Sequence Synthetic peptide
QRWGRGPGTT

SEQ ID NO: 200 - protein, 9 aa, Artificial Sequence
  Description of Artificial Sequence Synthetic peptide
DDSGIARDY

SEQ ID NO: 201 - protein, 10 aa, Artificial Sequence
  Description of Artificial Sequence Synthetic peptide
YRRFGINKNY

SEQ ID NO: 202 - protein, 13 aa, Artificial Sequence
  Description of Artificial Sequence Synthetic peptide
LRTYYLNDPVVFS

SEQ ID NO: 203 - protein, 11 aa, Artificial Sequence
  Description of Artificial Sequence Synthetic peptide
YIPLRGTLHDY

SEQ ID NO: 204 - protein, 7 aa, Artificial Sequence
  Description of Artificial Sequence Synthetic peptide
RKWGRNY

SEQ ID NO: 205 - protein, 11 aa, Artificial Sequence
  Description of Artificial Sequence Synthetic peptide
KRREWAKDFEY

SEQ ID NO: 206 - protein, 7 aa, Artificial Sequence
  Description of Artificial Sequence Synthetic peptide
IADWRGY

SEQ ID NO: 207 - protein, 11 aa, Artificial Sequence
  Description of Artificial Sequence Synthetic peptide
YIPYGGTLHDF

SEQ ID NO: 208 - protein, 7 aa, Artificial Sequence
  Description of Artificial Sequence Synthetic peptide
IADWRGY

SEQ ID NO: 209 - protein, 9 aa, Artificial Sequence
  Description of Artificial Sequence Synthetic peptide
DTIGTARDY

SEQ ID NO: 210 - protein, 9 aa, Artificial Sequence
  Description of Artificial Sequence Synthetic peptide
RTTSYPVDF

SEQ ID NO: 211 - protein, 11 aa, Artificial Sequence
  Description of Artificial Sequence Synthetic peptide
YIPYGGTLHDF

SEQ ID NO: 212 - protein, 3 aa, Artificial Sequence
  Description of Artificial Sequence Synthetic peptide
ASY

SEQ ID NO: 213 - protein, 3 aa, Artificial Sequence
  Description of Artificial Sequence Synthetic peptide
ASY

SEQ ID NO: 214 - protein, 11 aa, Artificial Sequence
  Description of Artificial Sequence Synthetic peptide
YIPYGGTLHDF

SEQ ID NO: 215 - protein, 11 aa, Artificial Sequence
  Description of Artificial Sequence Synthetic peptide
YIPYGGTLHDF

SEQ ID NO: 216 - protein, 11 aa, Artificial Sequence
  Description of Artificial Sequence Synthetic peptide
YIPYGGTLHDF

SEQ ID NO: 217 - protein, 9 aa, Artificial Sequence
  Description of Artificial Sequence Synthetic peptide
DTIGTARDY

SEQ ID NO: 218 - protein, 9 aa, Artificial Sequence
  Description of Artificial Sequence Synthetic peptide
DTIGTARDY

SEQ ID NO: 219 - protein, 9 aa, Artificial Sequence
  Description of Artificial Sequence Synthetic peptide
DTIGTARDY

SEQ ID NO: 220 - protein, 9 aa, Artificial Sequence
  Description of Artificial Sequence Synthetic peptide
RTTSYPVDF

SEQ ID NO: 221 - protein, 9 aa, Artificial Sequence
  Description of Artificial Sequence Synthetic peptide
RTTSYPVDF

SEQ ID NO: 222 - protein, 9 aa, Artificial Sequence
  Description of Artificial Sequence Synthetic peptide
RTTSYPVDF

SEQ ID NO: 223 - protein, 25 aa, Artificial Sequence
  Description of Artificial Sequence Synthetic peptide
QVQLVESGGGLVQPGGSLRLSCAAS

SEQ ID NO: 224 - protein, 25 aa, Artificial Sequence
  Description of Artificial Sequence Synthetic peptide
QVQLVESGGGLVQAGGSLRLSCAAS

SEQ ID NO: 225 - protein, 25 aa, Artificial Sequence
  Description of Artificial Sequence Synthetic peptide
QVQLVESGGGLVQAGGSLRLSCAFS

SEQ ID NO: 226 - protein, 25 aa, Artificial Sequence
  Description of Artificial Sequence Synthetic peptide
QVQLVESGGGLVQAGGSLRLSCVAS

SEQ ID NO: 227 - protein, 25 aa, Artificial Sequence
  Description of Artificial Sequence Synthetic peptide
QVQLVESGGGLVQAGGSLRLSCAAS

SEQ ID NO: 228 - protein, 25 aa, Artificial Sequence
  Description of Artificial Sequence Synthetic peptide
QVRLVESGGGLVQAGGSLRLSCAAS

SEQ ID NO: 229 - protein, 25 aa, Artificial Sequence
  Description of Artificial Sequence Synthetic peptide
QVRLVESGGGLVQAGESLRLSCAAS

SEQ ID NO: 230 - protein, 25 aa, Artificial Sequence
  Description of Artificial Sequence Synthetic peptide
QVQPVESGGGLVQPGGSLRLSCVVS

SEQ ID NO: 231 - protein, 25 aa, Artificial Sequence
  Description of Artificial Sequence Synthetic peptide
QVQPVESGGGLVQPGGSLRLSCVVS

SEQ ID NO: 232 - protein, 25 aa, Artificial Sequence
  Description of Artificial Sequence Synthetic peptide
EVQLVESGGGLVQPGNSLRLSCAAS

SEQ ID NO: 233 - protein, 25 aa, Artificial Sequence
  Description of Artificial Sequence Synthetic peptide
QVQIVESGGGLVQAGGSLRLSCVAS

SEQ ID NO: 234 - protein, 25 aa, Artificial Sequence
  Description of Artificial Sequence Synthetic peptide
QVQLVESGGGLVQTGGSLRLSCAAS

SEQ ID NO: 235 - protein, 25 aa, Artificial Sequence
  Description of Artificial Sequence Synthetic peptide
QVQLVESGGGLVQAGGSLRLSCAAS

SEQ ID NO: 236 - protein, 25 aa, Artificial Sequence
  Description of Artificial Sequence Synthetic peptide
QVQLVESGGGLVQAGGSLRLSCAAS

SEQ ID NO: 237 - protein, 25 aa, Artificial Sequence
  Description of Artificial Sequence Synthetic peptide
QVQLGESGGGLVQAGGSLRLSCAAS

SEQ ID NO: 238 - protein, 25 aa, Artificial Sequence
  Description of Artificial Sequence Synthetic peptide
QVQLVESGGGLVPSGGSLRLSCAAS

SEQ ID NO: 239 - protein, 25 aa, Artificial Sequence
  Description of Artificial Sequence Synthetic peptide
QVQLVESGGGLVQAGGSLRLSCAAS

SEQ ID NO: 240 - protein, 25 aa, Artificial Sequence
  Description of Artificial Sequence Synthetic peptide
QVQLVESGGGLVQAGESRRLSCAVS

SEQ ID NO: 241 - protein, 25 aa, Artificial Sequence
  Description of Artificial Sequence Synthetic peptide
QVQLVESGGGLVQAGGSLRLSCAAS

SEQ ID NO: 242 - protein, 25 aa, Artificial Sequence
  Description of Artificial Sequence Synthetic peptide
QVQLVESGGGLVQAGGSLRLSCAAS

SEQ ID NO: 243 - protein, 25 aa, Artificial Sequence
  Description of Artificial Sequence Synthetic peptide
QVQLVESGGGLVQAGGSLRLSCVAS

SEQ ID NO: 244 - protein, 25 aa, Artificial Sequence
  Description of Artificial Sequence Synthetic peptide
QVQLVESGGGLVQARGSLRLSCTAS

SEQ ID NO: 245 - protein, 25 aa, Artificial Sequence
  Description of Artificial Sequence Synthetic peptide
QVQLVESGGGLVQAGGSLRLSCAAS

SEQ ID NO: 246 - protein, 25 aa, Artificial Sequence
  Description of Artificial Sequence Synthetic peptide
QVQLVESGGGLVQAGGSLRLSCAAS

SEQ ID NO: 247 - protein, 25 aa, Artificial Sequence
  Description of Artificial Sequence Synthetic peptide
QVQLVESGGGLVQAGGSLRLSCTTS

SEQ ID NO: 248 - protein, 25 aa, Artificial Sequence
  Description of Artificial Sequence Synthetic peptide
QVQLVESGGGLVQAGGSLTLSCAAS

SEQ ID NO: 249 - protein, 25 aa, Artificial Sequence
  Description of Artificial Sequence Synthetic peptide
QVQLVESGGGLVQAGGSLRLSCVAS

SEQ ID NO: 250 - protein, 25 aa, Artificial Sequence
  Description of Artificial Sequence Synthetic peptide
QVQLVESGGGLVQAGGSLRLSCAAS

SEQ ID NO: 251 - protein, 25 aa, Artificial Sequence [sequence omitted]

SEQ ID NO: 252 - protein, 25 aa, Artificial Sequence [sequence omitted]

SEQ ID NO: 253 - protein, 25 aa, Artificial Sequence
  Description of Artificial Sequence Synthetic peptide
EVQLVESGGGLVQAGGSLRLSCAAS

SEQ ID NO: 254 - protein, 25 aa, Artificial Sequence [sequence omitted]

SEQ ID NO: 255 - protein, 25 aa, Artificial Sequence [sequence omitted]

SEQ ID NO: 256 - protein, 25 aa, Artificial Sequence
  Description of Artificial Sequence Synthetic peptide
QVQLVESGGGVVQAGGSLRLSCAAS

SEQ ID NO: 257 - protein, 25 aa, Artificial Sequence
  Description of Artificial Sequence Synthetic peptide
QVQLVESGGGVVQPGGSLRLSCAAS

SEQ ID NO: 258 - protein, 25 aa, Artificial Sequence
  Description of Artificial Sequence Synthetic peptide
QVQLVESGGGVVQPGGSLRLSCAAS

SEQ ID NO: 259 - protein, 25 aa, Artificial Sequence
  Description of Artificial Sequence Synthetic peptide
EVQLVESGGGLVQAGGSLRLSCVAS

SEQ ID NO: 260 - protein, 25 aa, Artificial Sequence [sequence omitted]

SEQ ID NO: 261 - protein, 25 aa, Artificial Sequence [sequence omitted]

SEQ ID NO: 262 - protein, 13 aa, Artificial Sequence
  Description of Artificial Sequence Synthetic peptide
WYRQAGNNRALVA

SEQ ID NO: 263 - protein, 14 aa, Artificial Sequence
  Description of Artificial Sequence Synthetic peptide
WYRQAPGKQRELVA

SEQ ID NO: 264 - protein, 14 aa, Artificial Sequence
  Description of Artificial Sequence Synthetic peptide
WYRQAPGKQRTVVA

SEQ ID NO: 265 - protein, 14 aa, Artificial Sequence
  Description of Artificial Sequence Synthetic peptide
WYRQAPGKERELVA

SEQ ID NO: 266 - protein, 14 aa, Artificial Sequence
  Description of Artificial Sequence Synthetic peptide
WYRQAPGKQRELVA

SEQ ID NO: 267 - protein, 14 aa, Artificial Sequence
  Description of Artificial Sequence Synthetic peptide
WYRQDPSKQREWVA

SEQ ID NO: 268 - protein, 14 aa, Artificial Sequence
  Description of Artificial Sequence Synthetic peptide
WYRQAPGKQRELVA

SEQ ID NO: 269 - protein, 14 aa, Artificial Sequence
  Description of Artificial Sequence Synthetic peptide
WYRQASGKERESVA

SEQ ID NO: 270 - protein, 14 aa, Artificial Sequence
  Description of Artificial Sequence Synthetic peptide
WYRQASGKERESVA

SEQ ID NO: 271 - protein, 14 aa, Artificial Sequence
  Description of Artificial Sequence Synthetic peptide
WVRQAPGKGLEWVS

SEQ ID NO: 272 - protein, 14 aa, Artificial Sequence
  Description of Artificial Sequence Synthetic peptide
WYRQAPGKEREMVA

SEQ ID NO: 273 - protein, 14 aa, Artificial Sequence
  Description of Artificial Sequence Synthetic peptide
WFRQAPGKQREWVA

SEQ ID NO: 274 - protein, 14 aa, Artificial Sequence
  Description of Artificial Sequence Synthetic peptide
WYRQAQGKQREPVA

SEQ ID NO: 275 - protein, 14 aa, Artificial Sequence
  Description of Artificial Sequence Synthetic peptide
WFRQAPGKERQFVA

SEQ ID NO: 276 - protein, 14 aa, Artificial Sequence
  Description of Artificial Sequence Synthetic peptide
WHRQAPGKQREPVA

SEQ ID NO: 277 - protein, 14 aa, Artificial Sequence
  Description of Artificial Sequence Synthetic peptide
WYRRAPGQVREMVA

SEQ ID NO: 278 - protein, 14 aa, Artificial Sequence
  Description of Artificial Sequence Synthetic peptide
WYRQAPGTERDLVA

SEQ ID NO: 279 - protein, 14 aa, Artificial Sequence
  Description of Artificial Sequence Synthetic peptide
WYRQAPGAQRELLA

SEQ ID NO: 280 - protein, 14 aa, Artificial Sequence
  Description of Artificial Sequence Synthetic peptide
WYRQAPGKQRELVA

SEQ ID NO: 281 - protein, 14 aa, Artificial Sequence
  Description of Artificial Sequence Synthetic peptide
WYRQAPGKERELVA

SEQ ID NO: 282 - protein, 14 aa, Artificial Sequence
  Description of Artificial Sequence Synthetic peptide
WYRQAPGKQRELVA

SEQ ID NO: 283 - protein, 14 aa, Artificial Sequence
  Description of Artificial Sequence Synthetic peptide
WYRQAPGNQRELVA

SEQ ID NO: 284 - protein, 14 aa, Artificial Sequence
  Description of Artificial Sequence Synthetic peptide
WYRQAPGKQREPVA

SEQ ID NO: 285 - protein, 14 aa, Artificial Sequence
  Description of Artificial Sequence Synthetic peptide
WFRQAPGEERELVA

SEQ ID NO: 286 - protein, 14 aa, Artificial Sequence
  Description of Artificial Sequence Synthetic peptide
WYRQAPGNQREPVA

SEQ ID NO: 287 - protein, 14 aa, Artificial Sequence
  Description of Artificial Sequence Synthetic peptide
WYRQAPGTERDLVA

SEQ ID NO: 288 - protein, 14 aa, Artificial Sequence
  Description of Artificial Sequence Synthetic peptide
WHRQAPGNERELVA

SEQ ID NO: 289 - protein, 14 aa, Artificial Sequence
  Description of Artificial Sequence Synthetic peptide
WYRQAPGKERELVA

SEQ ID NO: 290 - protein, 14 aa, Artificial Sequence
  Description of Artificial Sequence Synthetic peptide
WYRQAPGKQRELVA

SEQ ID NO: 291 - protein, 14 aa, Artificial Sequence
  Description of Artificial Sequence Synthetic peptide
WVRQAPGKGLEWVS

SEQ ID NO: 292 - protein, 14 aa, Artificial Sequence
  Description of Artificial Sequence Synthetic peptide
WYRQAPGKERELVA

SEQ ID NO: 293 - protein, 14 aa, Artificial Sequence
  Description of Artificial Sequence Synthetic peptide
WYRQAPGKERELVA

SEQ ID NO: 294 - protein, 14 aa, Artificial Sequence
  Description of Artificial Sequence Synthetic peptide
WVRQAPGKGLEWVS

SEQ ID NO: 295 - protein, 14 aa, Artificial Sequence
  Description of Artificial Sequence Synthetic peptide
WYRQAPGTERDLVA

SEQ ID NO: 296 - protein, 14 aa, Artificial Sequence
  Description of Artificial Sequence Synthetic peptide
WYRQAPGKERELVA

SEQ ID NO: 297 - protein, 14 aa, Artificial Sequence
  Description of Artificial Sequence Synthetic peptide
WVRQAPGKGLEWVS

SEQ ID NO: 298 - protein, 14 aa, Artificial Sequence
  Description of Artificial Sequence Synthetic peptide
WHRQAPGNERELVA

SEQ ID NO: 299 - protein, 14 aa, Artificial Sequence
  Description of Artificial Sequence Synthetic peptide
WHRQAPGKERELVA

SEQ ID NO: 300 - protein, 14 aa, Artificial Sequence
  Description of Artificial Sequence Synthetic peptide
WVRQAPGKGLEWVS

SEQ ID NO: 301 - protein, 29 aa, Artificial Sequence
  Description of Artificial Sequence Synthetic peptide
ISWDIAENTVYLQMNSLNSEDTTVYYCNS

SEQ ID NO: 302 - protein, 29 aa, Artificial Sequence
  Description of Artificial Sequence Synthetic peptide
ISGDNVRNMVYLQMNSLKPEDTAIYYCSA

SEQ ID NO: 303 - protein, 29 aa, Artificial Sequence
  Description of Artificial Sequence Synthetic peptide
ISGENGKNTVYLQMNSLKLEDTAVYYCLG

SEQ ID NO: 304 - protein, 29 aa, Artificial Sequence
  Description of Artificial Sequence Synthetic peptide
ISRDNANNAIYLEMNSLKPEDTAVYVCNA

SEQ ID NO: 305 - protein, 29 aa, Artificial Sequence
  Description of Artificial Sequence Synthetic peptide
ISRDNAENTVSLQMNTLKPEDTAVYFCNA

SEQ ID NO: 306 - protein, 29 aa, Artificial Sequence
  Description of Artificial Sequence Synthetic peptide
ISKDSTRNTVYLQMNMLRPEDTAVYYCNA

SEQ ID NO: 307 - protein, 29 aa, Artificial Sequence
  Description of Artificial Sequence Synthetic peptide
ISRDNAKNTVYLQMNSLKPEDTAVYYCNA

SEQ ID NO: 308 - protein, 29 aa, Artificial Sequence
  Description of Artificial Sequence Synthetic peptide
ISRDIDKKTVYLQMDNLKPEDTGVYYCNS

SEQ ID NO: 309 - protein, 29 aa, Artificial Sequence
  Description of Artificial Sequence Synthetic peptide
ISRDIYKKTVYLQMDNLKPEDTGVYYCNS

SEQ ID NO: 310 - protein, 29 aa, Artificial Sequence
  Description of Artificial Sequence Synthetic peptide
ISRDNAKTTLYLQMNSLRPEDTAVYYCTI

SEQ ID NO: 311 - protein, 29 aa, Artificial Sequence
  Description of Artificial Sequence Synthetic peptide
ISKENAKNTVYLQMNSLKPEDTAVYYCHI

SEQ ID NO: 312 - protein, 29 aa, Artificial Sequence
  Description of Artificial Sequence Synthetic peptide
ISRDNAKSTVYLQMNSLKPEDTAIYYCKA

SEQ ID NO: 313 - protein, 29 aa, Artificial Sequence
  Description of Artificial Sequence Synthetic peptide
ISRDNAKNTVYLQMNSLKPEDTAIYTCHV

SEQ ID NO: 314 - protein, 29 aa, Artificial Sequence
  Description of Artificial Sequence Synthetic peptide
ISRDNAANTFYLQMNNLRPDDTAVYYCNV

SEQ ID NO: 315 - protein, 29 aa, Artificial Sequence
  Description of Artificial Sequence Synthetic peptide
VSRDIVKNTMYLQMNSLKPEDTAVYYCSY

SEQ ID NO: 316 - protein, 29 aa, Artificial Sequence
  Description of Artificial Sequence Synthetic peptide
ISRDNTQNLVYLQMNNLQPHDTAIYYCGA

SEQ ID NO: 317 - protein, 29 aa, Artificial Sequence
  Description of Artificial Sequence Synthetic peptide
ISRDNAKNTLYLQMNNLKPEDTAVYYCNA

SEQ ID NO: 318 - protein, 29 aa, Artificial Sequence
  Description of Artificial Sequence Synthetic peptide
ISRDNAKNTVYLQMNRLTPEDTDVYYCRF

SEQ ID NO: 319 - protein, 29 aa, Artificial Sequence
  Description of Artificial Sequence Synthetic peptide
ISRDNAKNTVYLQMESLVAEDTAVYYCNA

SEQ ID NO: 320 - protein, 29 aa, Artificial Sequence
  Description of Artificial Sequence Synthetic peptide
VSRDSAKNIVYLQMNSLTPEDTAVYYCNT

SEQ ID NO: 321 - protein, 29 aa, Artificial Sequence
  Description of Artificial Sequence Synthetic peptide
ISRDNAKNTVYLQMNSLKPEDTAVYYCNA

SEQ ID NO: 322 - protein, 29 aa, Artificial Sequence
  Description of Artificial Sequence Synthetic peptide
ISRDNAKNTVYLQMNSLKPEDTAVYYCNT

SEQ ID NO: 323 - protein, 29 aa, Artificial Sequence
  Description of Artificial Sequence Synthetic peptide
ISRDNAKNTVYLQMNSLKPEDTAIYTCHV

SEQ ID NO: 324 - protein, 29 aa, Artificial Sequence
  Description of Artificial Sequence Synthetic peptide
VSRDSAKNIVYLQMNRLKPEDTAVYYCNT

SEQ ID NO: 325 - protein, 29 aa, Artificial Sequence
  Description of Artificial Sequence Synthetic peptide
ISRDNARNTVYLQMDSLKPEDTAIYTCHV

SEQ ID NO: 326 - protein, 29 aa, Artificial Sequence
  Description of Artificial Sequence Synthetic peptide
ISRDNAKNTLYLQMNNLKPEDTAVYYCNA

SEQ ID NO: 327 - protein, 29 aa, Artificial Sequence
  Description of Artificial Sequence Synthetic peptide
ISRDNAKNTAYLQMNSLKPEDTAVYYCSV

SEQ ID NO: 328 - protein, 29 aa, Artificial Sequence
  Description of Artificial Sequence Synthetic peptide
VSRDSAKNIVYLQMNSLKPEDTAVYYCNT

SEQ ID NO: 329 - protein, 29 aa, Artificial Sequence
  Description of Artificial Sequence Synthetic peptide
ISRDNSKNTLYLQMNSLRAEDTAVYYCAV

SEQ ID NO: 330 - protein, 29 aa, Artificial Sequence
  Description of Artificial Sequence Synthetic peptide
ISRDNSKNTLYLQMNSLRAEDTAVYYCAV

SEQ ID NO: 331 - protein, 29 aa, Artificial Sequence
  Description of Artificial Sequence Synthetic peptide
ISRDNSKNTLYLQMNSLRAEDTAVYYCNT

SEQ ID NO: 332 - protein, 29 aa, Artificial Sequence
  Description of Artificial Sequence Synthetic peptide
ISRDNSKNTLYLQMNSLRAEDTAVYYCNT

SEQ ID NO: 333 - protein, 29 aa, Artificial Sequence
  Description of Artificial Sequence Synthetic peptide
ISRDNSKNTLYLQMNSLRAEDTAVYYCNT

SEQ ID NO: 334 - protein, 29 aa, Artificial Sequence
  Description of Artificial Sequence Synthetic peptide
ISRDNSKNTLYLQMNSLRAEDTAVYYCNA

SEQ ID NO: 335 - protein, 29 aa, Artificial Sequence
  Description of Artificial Sequence Synthetic peptide
ISRDNSKNTLYLQMNSLRAEDTAVYYCNA

SEQ ID NO: 336 - protein, 29 aa, Artificial Sequence
  Description of Artificial Sequence Synthetic peptide
ISRDNSKNTLYLQMNSLRAEDTAVYYCNA

SEQ ID NO: 337 - protein, 29 aa, Artificial Sequence
  Description of Artificial Sequence Synthetic peptide
ISRDNSKNTLYLQMNSLRAEDTAVYYCSV

SEQ ID NO: 338 - protein, 29 aa, Artificial Sequence
  Description of Artificial Sequence Synthetic peptide
ISRDNSKNTLYLQMNSLRAEDTAVYYCSV

SEQ ID NO: 339 - protein, 29 aa, Artificial Sequence
  Description of Artificial Sequence Synthetic peptide
ISRDNSKNTLYLQMNSLRAEDTAVYYCSV

SEQ ID NO: 340 - protein, 11 aa, Artificial Sequence
  Description of Artificial Sequence Synthetic peptide
WGQGTQVTVSS

SEQ ID NO: 341 - protein, 11 aa, Artificial Sequence
  Description of Artificial Sequence Synthetic peptide
WGKGTLVTVSS

SEQ ID NO: 342 - protein, 11 aa, Artificial Sequence
  Description of Artificial Sequence Synthetic peptide
WGQGTQVTVSS

SEQ ID NO: 343 - protein, 11 aa, Artificial Sequence
  Description of Artificial Sequence Synthetic peptide
FGQGTQVTVSS

SEQ ID NO: 344 - protein, 11 aa, Artificial Sequence
  Description of Artificial Sequence Synthetic peptide
WGQGTQVTVSS

SEQ ID NO: 345 - protein, 11 aa, Artificial Sequence
  Description of Artificial Sequence Synthetic peptide
WGQGTQVTVSS

SEQ ID NO: 346 - protein, 11 aa, Artificial Sequence
  Description of Artificial Sequence Synthetic peptide
WGQGTQVTVSS

SEQ ID NO: 347 - protein, 11 aa, Artificial Sequence
  Description of Artificial Sequence Synthetic peptide
WGQGTQVTVSS

SEQ ID NO: 348 - protein, 11 aa, Artificial Sequence
  Description of Artificial Sequence Synthetic peptide
WGQGTQVTVSS

SEQ ID NO: 349 - protein, 9 aa, Artificial Sequence
  Description of Artificial Sequence Synthetic peptide
QGTLVTVSS

SEQ ID NO: 350 - protein, 11 aa, Artificial Sequence
  Description of Artificial Sequence Synthetic peptide
WGQGTQVTVSS

SEQ ID NO: 351 - protein, 11 aa, Artificial Sequence
  Description of Artificial Sequence Synthetic peptide
WGQGTQVTVSS

SEQ ID NO: 352 - protein, 11 aa, Artificial Sequence
  Description of Artificial Sequence Synthetic peptide
WGQGTQVTVSS

SEQ ID NO: 353 - protein, 11 aa, Artificial Sequence
  Description of Artificial Sequence Synthetic peptide
WGQGTQVTVSS

SEQ ID NO: 354 - protein, 11 aa, Artificial Sequence
  Description of Artificial Sequence Synthetic peptide
WGQGTQVTVSS

SEQ ID NO: 355 - protein, 11 aa, Artificial Sequence
  Description of Artificial Sequence Synthetic peptide
WGQGTQVTVSS

SEQ ID NO: 356 - protein, 11 aa, Artificial Sequence
  Description of Artificial Sequence Synthetic peptide
WGQGTQVTVSS

SEQ ID NO: 357 - protein, 11 aa, Artificial Sequence
  Description of Artificial Sequence Synthetic peptide
WGQGTQVTVSS

SEQ ID NO: 358 - protein, 11 aa, Artificial Sequence
  Description of Artificial Sequence Synthetic peptide
WGQGTQVTVSS

SEQ ID NO: 359 - protein, 11 aa, Artificial Sequence
  Description of Artificial Sequence Synthetic peptide
WGQGTQVTVSS

SEQ ID NO: 360 - protein, 11 aa, Artificial Sequence
  Description of Artificial Sequence Synthetic peptide
WGQGTQVTVSS

SEQ ID NO: 361 - protein, 11 aa, Artificial Sequence
  Description of Artificial Sequence Synthetic peptide
WGQGTQVTVSS

SEQ ID NO: 362 - protein, 11 aa, Artificial Sequence
  Description of Artificial Sequence Synthetic peptide
WGQGTQVTVSS

SEQ ID NO: 363 - protein, 11 aa, Artificial Sequence
  Description of Artificial Sequence Synthetic peptide
WGQGTQVTVSS

SEQ ID NO: 364 - protein, 11 aa, Artificial Sequence
  Description of Artificial Sequence Synthetic peptide
WGQGTQVTVSS

SEQ ID NO: 365 - protein, 11 aa, Artificial Sequence
  Description of Artificial Sequence Synthetic peptide
WGQGTQVTVSS

SEQ ID NO: 366 - protein, 11 aa, Artificial Sequence
  Description of Artificial Sequence Synthetic peptide
WGQGTQVTVSS

SEQ ID NO: 367 - protein, 11 aa, Artificial Sequence
  Description of Artificial Sequence Synthetic peptide
WGQGTQVTVSS

SEQ ID NO: 368 - protein, 11 aa, Artificial Sequence
  Description of Artificial Sequence Synthetic peptide
WGQGTLVTVSS

SEQ ID NO: 369 - protein, 11 aa, Artificial Sequence
  Description of Artificial Sequence Synthetic peptide
WGQGTLVTVSS

SEQ ID NO: 370 - protein, 11 aa, Artificial Sequence
  Description of Artificial Sequence Synthetic peptide
WGQGTLVTVSS

SEQ ID NO: 371 - protein, 11 aa, Artificial Sequence
  Description of Artificial Sequence Synthetic peptide
WGQGTLVTVSS

SEQ ID NO: 372 - protein, 11 aa, Artificial Sequence
  Description of Artificial Sequence Synthetic peptide
WGQGTLVTVSS

SEQ ID NO: 373 - protein, 13 aa, Artificial Sequence
  Description of Artificial Sequence Synthetic peptide
WGQGTLVTVSSGG

SEQ ID NO: 374 - protein, 13 aa, Artificial Sequence
  Description of Artificial Sequence Synthetic peptide
WGQGTLVTVSSGG

SEQ ID NO: 375 - protein, 13 aa, Artificial Sequence
  Description of Artificial Sequence Synthetic peptide
WGQGTLVTVSSGG

SEQ ID NO: 376 - protein, 10 aa, Artificial Sequence
  Description of Artificial Sequence Synthetic peptide
WGQGTLVTVS

SEQ ID NO: 377 - protein, 11 aa, Artificial Sequence
  Description of Artificial Sequence Synthetic peptide
WGQGTLVTVSS

SEQ ID NO: 378 - protein, 11 aa, Artificial Sequence
  Description of Artificial Sequence Synthetic peptide
WGQGTLVTVSS

SEQ ID NO: 379 - protein, 6 aa, Artificial Sequence
  Description of Artificial Sequence Synthetic 6xHis tag
HHHHHH